(19) United States
(12) Patent Application Publication
(10) Pub. No.: US 2024/0131124 A1
(43) Pub. Date: Apr. 25, 2024

(54) ANTIVIRAL STRUCTURALLY-STABILIZED ACE2 HELIX 1 PEPTIDES AND USES THEREOF

(71) Applicant: Dana-Farber Cancer Institute, Inc., Boston, MA (US)

(72) Inventors: Loren D. Walensky, Newton, MA (US); Henry D. Herce, Brookline, MA (US)

(21) Appl. No.: 17/919,008

(22) PCT Filed: Apr. 22, 2021
(86) PCT No.: PCT/US2021/028609

§ 371 Date: Oct. 14, 2022

Related U.S. Application Data
(60) Provisional application No. 63/014,042, filed on Apr. 22, 2020.

(51) Int. Cl. A61K 38/48; A61P 31/14; C12N 9/48; G01N 33/569

(52) U.S. Cl. CPC A61K 38/4813; A61P 31/14; C12N 9/485; C12Y 304/17023; G01N 33/56983; G01N 2333/165; G01N 2469/10

(57) ABSTRACT

Disclosed herein are structurally stabilized peptides of ACE2 helix 1 useful for diagnosing, preventing, and treating coronavirus infection by targeting the receptor binding domain of SARS-CoV-2 and thereby blocking its interaction with the human ACE2 receptor, which is involved in coronavirus infection and pathogenesis.

### CROSS-REFERENCE TO RELATED APPLICATION

This application claims the benefit of priority to U.S. Provisional Appl. No. 63/014,042, filed Apr. 22, 2020, the contents of which are incorporated by reference in their entirety herein.

### SEQUENCE LISTING

This application contains a Sequence Listing that has been submitted electronically as an ASCII text file named “00530-0408US1_SL_ST25.txt.” The ASCII text file, created on May 10, 2023, is 130,605 bytes in size. The material in the ASCII text file is hereby incorporated by reference in its entirety.

### TECHNICAL FIELD

This disclosure relates to structurally-stabilized antiviral peptides based on inhibiting the interaction between ACE2 helix 1 and the receptor-binding domain of SARS-CoV-2 type viruses, diagnostic reagents, and methods for using such peptides and reagents in the diagnosis, prevention, and treatment of a coronavirus infection.

### BACKGROUND

To date, no anti-viral therapeutic currently exists to prevent or treat infection by novel coronavirus (nCoV) outbreaks, such as the SARS-CoV-2 (COVID-19, also known as 2019-nCoV). SARS-CoV-2 has been declared a high-risk global health emergency by the World Health Organization (WHO) and has, as of March 2021, caused 114,857,764 cases of respiratory disease and 2,551,459 deaths worldwide.

New strategies for the prophylaxis and/or treatment of SARS-CoV-2 infection are urgently required to effectively mitigate the outbreak.

### SUMMARY

Disclosed herein is a druggable protein-interaction mechanism ideally suited for targeted inhibition of the virus using stapled peptide technology. Featured are stapled peptide inhibitors of the SARS-CoV-2/ACE2 interaction for the diagnosis, prevention, and treatment of COVID-19.

This application relates to compositions and methods based on peptide structure-stabilizing technology (e.g., stapling, stitching) that recapitulates and fortifies the structure of bioactive helices to generate a targeted prophylactic and therapeutic agent for prevention and/or treatment of a coronaviruses that infect host cells by engaging the human angiotensin converting enzyme 2 (ACE2) receptor (e.g., betacoronavirus such as SARS-CoV-2) infection. By inserting “staples” (e.g., all-hydrocarbon staples) or “stitches” into natural peptides, bioactive-helical structure is restored and, thus remarkable protease resistance is conferred by burying the otherwise labile amide bonds at the core of the helical structure. Here, structurally-stabilized peptide inhibitors of coronavirus (e.g., betacoronavirus such as SARS-CoV-2) are disclosed. These structurally-stabilized peptide inhibitors are used to treat and/or prevent and/or diagnose coronavirus (e.g., betacoronavirus such as SARS-CoV-2) infection. These structurally-stabilized peptide inhibitors are used to treat and/or prevent and/or diagnose SARS-CoV-2 variants (e.g., B.1.1.7, B.1.351, P.1, B.1.427, B.1.429, B.1.167). These structurally-stabilized peptide inhibitors are used to treat and/or prevent and/or diagnose infection by any virus (including a coronavirus) that requires ACE2 receptor binding as part of its infection. One form of structurally-stabilized peptides is a stapled peptide. Stapled peptides reinforce the natural alpha-helical shape of bioactive peptides, conferring stabilized structure, protease resistance (to prevent peptide degradation in the blood or tissues), enhanced target binding affinity, and favorable pharmacology. Walensky and Bird, J. Med. Chem., 57, 6275-88 (2014); Walensky et al., Science, 305, 1466-70 (2004).

Angiotensin-converting enzyme 2 (ACE2) is the cellular receptor for SARS-CoV-2. In particular, this virus binds through its receptor binding domain (SARS-CoV-2 RBD) to an alpha-helical peptide (al helix) of the ACE2 receptor on the surface of a cell (e.g., a respiratory epithelial cell). The present invention describes peptide analogs of the al helix of ACE2 that bind and thus inhibit the ACE2-SARS-CoV-2 virus RBD interaction, thereby preventing or inhibiting coronavirus infection. A stabilized peptide inhibitor of the SARS-CoV-2/ACE2 interaction can be utilized in the prevention and treatment of COVID-19. In addition, the capacity of the peptide analogs to specifically bind to the SARS-CoV-2 surface enables their use as detection reagents for the virus in human and animal samples, and thus serve as a diagnostic of SARS-CoV-2 infection.

The disclosure features a structurally-stabilized alpha-helical peptide (al helix) of the ACE2 receptor (e.g., human ACE2 receptor) that blocks or inhibits interaction between ACE2 (e.g., human ACE2) and the RBD of a virus that binds ACE2. In some instances, the structural stabilization of the peptide is by stapling (e.g., hydrocarbon stapling). In some instances, the stabilization is by stitching. In some instances, the stabilization is by a lactam staple or stitch; a UV-cycloaddition staple or stitch; an oxime staple or stitch; a thioether staple or stitch; a double-click staple or stitch; a bis-lactam staple or stitch; a bis-arylation staple or stitch; or a combination of any two or more thereof. In some cases, the virus is a coronavirus (e.g., alphacoronavirus, betacoronavirus). In certain cases, the virus is SARS-CoV-1, SARS-CoV-2, or HCoV-NL63.

This disclosure features a polypeptide comprising an amino acid sequence that is at least 30% and less than 81% identical to EQAKTFLDKFNHEAEDLFYQ (SEQ ID NO:77). In some instances, the polypeptide is at least 35%, 40%, 45%, 50%, 55%, 60%, 65%, 70%, 75%, or 80% identical to SEQ ID NO:77. In some instances, these polypeptides are also less than 61%, 71%, or 81% identical to SEQ ID NO:77. In some instances, the polypeptide has at least 4, at least 5, at least 6, at least 7, at least 8, at least 9, at least 10, at least 11, at least 12, at least 13, or at least 14 amino acid substitutions relative to EQAKTFLDKFNHEAEDLFYQ (SEQ ID NO:77). In some instances, these polypeptides do not have more than 14 amino acid substitutions relative to SEQ ID NO:77. In some instances, these polypeptides do not have more than 7, 8, 9, 10, 11, 12, or 13 amino acid substitutions relative to SEQ ID NO:77.

In some instances, the polypeptides described herein have one or more (e.g., 1, 2, 3, 4, 5, 6, 7) of the following properties: (i) binds the peptide of SEQ ID NO: 64 or SEQ ID NO: 65; (ii) inhibits interaction between human ACE2 protein and the S1 protein subunit of SARS-CoV-2; (iii) inhibits interaction between the carboxypeptidase domain of human ACE2 protein and the S1 protein subunit of SARS-CoV-2; (iv) inhibits interaction between the carboxypeptidase domain of human ACE2 protein and the RBD of the S1 protein subunit of SARS-CoV-2; (v) competes for human ACE2-SARS-CoV-2 S1 protein subunit binding; (vi) binds the S1 protein subunit (e.g., the RBD) of a SARS-CoV-2 variant (e.g., one or more of B.1.1.7, B.1.351, P.1, B.1.427, B.1.429, B.1.167); and (vii) inhibits SARS virus infection, optionally, wherein the SARS infection is SARS-CoV-2 infection. The stabilized (e.g., stapled/stitched) forms of the polypeptide have enhanced alpha-helical structure and/or increased protease resistance relative to the native template sequence (i.e., the sequence without the internal cross-link(s)).

In one aspect, this disclosure features an internally cross-linked ACE2h1 peptide that binds to both the S1 protein and/or RBD of SARS-CoV-2 and the S1 protein and/or RBD of one or more SARS-CoV-2 variants, optionally wherein the SARS-CoV-2 variant B.1.1.7, B.1.351, P.1, B.1.427, B.1.429, or B.1.167. In some instances, the internally cross-linked peptide has an amino acid sequence that differs from any one of SEQ ID NOs.: 77, 90-95, 98-100, 105-108, 110, 112, 113, 117, 118, 123, 125, or 127, or 145-148 at 2, 3, 4, 5, 6, 7, or 8 amino acid positions. In some instances, the internally cross-linked peptide comprises α, α-disubstituted non-natural amino acids with olefinic side chains that are internally cross-linked, wherein the α, α-disubstituted non-natural amino acids are inserted at one or more of (i)-(vi): (i) positions 5 and 12, (ii) positions 11 and 18, (iii) positions 12 and 19, (iv) positions 14 and 18, (v) positions 15 and 19, or (vi) positions 16 and 20, wherein the position numbering is provided based on the N-terminal E (position 1) to the C-terminal Q (position 20) of SEQ ID NO:77. In some cases, the peptides is 20 to 100 amino acids in length. In some cases, the peptides is 20 to 75 amino acids in length. In some cases, the peptides is 20 to 50 amino acids in length. In some cases, the peptides is 20 to 40 amino acids in length. In some cases, the peptides is 20 to 30 amino acids in length. In some cases, the peptides is 20 to 25 amino acids in length. Also featured are pharmaceutical compositions comprising the above internally cross-linked peptide and a pharmaceutically acceptable carrier. These internally cross-linked peptides can be used to treat or prevent a coronavirus infection (e.g., a betacoronavirus infection such as SARS-CoV-1, SARS-CoV-2, HCoV-NL63). These internally cross-linked peptides can be used to treat or prevent post-acute sequelae of SARS-CoV-2. These internally cross-linked peptides are especially useful to treat or prevent infection by variants of SARS-CoV-2.

In some instances, the polypeptide is at least 30% and less than 71% identical to SEQ ID NO:77. In some instances, the polypeptide is at least 40% and less than 71% identical to SEQ ID NO:77. In some instances, the polypeptide is at least 50% and less than 71% identical to SEQ ID NO:77. In some instances, the polypeptide is at least 60% and less than 71% identical to SEQ ID NO:77. In some instances, the polypeptide is at least 65% and less than 71% identical to SEQ ID NO:77. In some instances, the polypeptide is at least 30% and less than 61% identical to SEQ ID NO:77. In some instances, the polypeptide is at least 40% and less than 61% identical to SEQ ID NO:77. In some instances, the polypeptide is at least 50% and less than 61% identical to SEQ ID NO:77. In some instances, the polypeptide is at least 60% and less than 61% identical to SEQ ID NO:77.

In some instances, the polypeptide comprises the amino acid sequence selected from:

(i)
(SEQ ID NO: 125)
QEEQAKDAADHANHEAEYQAYQSA,
(ii)
(SEQ ID NO: 113)
IEEQAKTAADKANHEAEDAAYQSA,
(iii)
(SEQ ID NO: 118)
IEEQAKTAADKANHEAEQAAYQSA,
(iv)
(SEQ ID NO: 117)
AEEQAKTAADKAAHEAEQAAYQAA,
(v)
(SEQ ID NO: 123)
IQEQAKTDADKHNHEAEDYQYQSA,
(vi)
(SEQ ID NO: 127)
ETVDFFAEWFDVEAEDKDYL,
or
(vii)
(SEQ ID NO: 112)
IEEQAKTFLDKFNHEAEDLFYQSA.

In some instances, the above polypeptide includes 0, at least 1, at least 2, at least 3, at least 4, or at least 5 amino acid substitutions in any amino acid sequence of (i)-(vi). In some instances, the polypeptide includes 0, 1, 2, 3, 4, 5, or more amino acid substitutions in any amino acid sequence of (i)-(vi). In some instances, the polypeptide includes no more than 2, 3, 4, 5, 6, 7, 8, or 9 amino acid substitutions relative to an amino acid sequence of (i)-(vi). In some instances, any of the amino acid sequences of (i)-(vi) includes at least two, three, or four amino acid substitutions that are with α, α-disubstituted non-natural amino acids with olefinic side chains that can be internally cross-linked and wherein the at least two, three, or four amino acid substitutions are at positions 2, 3, or 6 amino acids apart. In some instances, the polypeptide of (i)-(vi) or a variant described herein has one or more (e.g., 1, 2, 3, 4, 5, 6, 7) of the following properties: (i) binds the peptide of SEQ ID NO: 64 or SEQ ID NO: 65; (ii) inhibits interaction between human ACE2 protein and the S1 protein subunit of SARS-CoV-2; (iii) inhibits interaction between the carboxypeptidase domain of human ACE2 protein and the S1 protein subunit of SARS-CoV-2; (iv) inhibits interaction between the carboxypeptidase domain of human ACE2 protein and the RBD of the S1 protein subunit of SARS-CoV-2; (v) competes for human ACE2-SARS-CoV-2 S1 protein subunit binding; (vi) binds the S1 protein subunit (e.g., the RBD) of a SARS-CoV-2 variant (e.g., one or more of B.1.1.7, B.1.351, P.1, B.1.427, B.1.429, B.1.167); and (vii) inhibits SARS virus infection, optionally, wherein the SARS infection is SARS-CoV-2 infection.

Also featured herein are polypeptides comprising the amino acid sequence of any one of the sequences set forth in SEQ ID NOs.: 90-95, 98-100, 105-108, and 110, with 0, 1, 2, 3, 4, 5, 6, 7, 8, 9, 10, 11, or 12 amino acid substitutions. In some cases, the non-natural amino acids that are present in these sequences are not substituted. In some instances, the disclosure features a peptide comprising a sequence selected from any one of SEQ ID NOs: 78-111. In some instances, the polypeptides of SEQ ID NOs: 78-111 include one or more (e.g., at least one, at least two, at least three, at least four, at least five, at least six, at least seven, at least eight, at least nine, at least ten, at least eleven, or at least twelve) additional amino acid substitutions compared to IEEQAKTFLDKFNHEAEDLFYQS (SEQ ID NO:76). In some instances, these polypeptides of SEQ ID NOs: 78-111 include no more than 6, 7, 8, 9, 10, 11, or 12 amino acid substitutions. In some instances, the polypeptide is at least 30% and less than 90% identical to SEQ ID NO:76. In some instances, the polypeptide is at least 40% and less than 90% identical to SEQ ID NO:76. In some instances, the polypeptide is at least 50% and less than 90% identical to SEQ ID NO:76. In some instances, the polypeptide is at least 60% and less than 90% identical to SEQ ID NO:76. In some instances, the polypeptide is at least 65% and less than 90% identical to SEQ ID NO:76. In some instances, the polypeptide is at least 30%, at least 35%, at least 40%, at least 45%, at least 50%, at least 55%, at least 60%, at least 65%, at least 70%, at least 75%, at least 80%, at least 85%, or at least 90% identical to SEQ ID NO: 76. These polypeptides have one or more (e.g., 1, 2, 3, 4, 5, 6, 7) of the following properties: (i) binds the peptide of SEQ ID NO: 64 or SEQ ID NO: 65; (ii) inhibits interaction between human ACE2 protein and the S1 protein subunit of SARS-CoV-2; (iii) inhibits interaction between the carboxypeptidase domain of human ACE2 protein and the S1 protein subunit of SARS-CoV-2; (iv) inhibits interaction between the carboxypeptidase domain of human ACE2 protein and the RBD of the S1 protein subunit of SARS-CoV-2; (v) competes for human ACE2-SARS-CoV-2 S1 protein subunit binding; (vi) binds the S1 protein subunit (e.g., the RBD) of a SARS-CoV-2 variant (e.g., one or more of B.1.1.7, B.1.351, P.1, B.1.427, B.1.429, B.1.167); and (vii) inhibits SARS virus infection, optionally, wherein the SARS infection is SARS-CoV-2 infection.

In some instances, any of the polypeptides described herein is stabilized (i.e., the alpha helical structure of the peptide is stabilized by any means known in the art). The stabilized polypeptides have increased alpha helicity and/or protease resistance relative to the uncross-linked or template sequence. In some instances, stabilization of protein in performed using triazole stapling or hydrogen bond surrogate (HBS) stabilization. In some instances, the polypeptide is hydrocarbon stapled or stitched. In some instances, the polypeptide is not stapled or stitched. In some instances, the polypeptide comprises an amino acid sequence set forth in any one of SEQ ID NOs: 10-12, 17-20, and 113-133. In some instances, the polypeptide is at least 30%, at least 35%, at least 40%, at least 45%, at least 50%, at least 55%, at least 60%, at least 65%, at least 70%, at least 75%, at least 80%, at least 85%, or at least 90% identical to any one of SEQ ID NOs: 10-12, 17-20, and 113-133. In some instances, the polypeptide is less than at least 71%, 76%, 81%, 86%, or 91% identical to any one of SEQ ID NOs: 10-12, 17-20, and 113-133. These polypeptides have one or more (e.g., 1, 2, 3, 4, 5, 6, 7) of the following properties: (i) binds the peptide of SEQ ID NO: 64 or SEQ ID NO: 65; (ii) inhibits interaction between human ACE2 protein and the S1 protein subunit of SARS-CoV-2; (iii) inhibits interaction between the carboxypeptidase domain of human ACE2 protein and the S1 protein subunit of SARS-CoV-2; (iv) inhibits interaction between the carboxypeptidase domain of human ACE2 protein and the RBD of the S1 protein subunit of SARS-CoV-2; (v) competes for human ACE2-SARS-CoV-2 S1 protein subunit binding; (vi) binds the S1 protein subunit (e.g., the RBD) of a SARS-CoV-2 variant (e.g., one or more of B.1.1.7, B.1.351, P.1, B.1.427, B.1.429, B.1.167); and (vii) inhibits SARS virus infection, optionally, wherein the SARS infection is SARS-CoV-2 infection.

In some instances, the polypeptide comprises any one of the amino acid sequences:

(i)
(SEQ ID NO: 115)
IEEQXKTAXDKANHEXEDAXYQSA,
(ii)
(SEQ ID NO: 116)
IEEQXKEAXDKANHEXEDAXYQSA,
(iii)
(SEQ ID NO: 120)
IEEQXKTAXDKANHEXEQAXYQSA,
(iv)
(SEQ ID NO: 121)
IEEQXKEAXDKANHEXEQAXYQSA,
(v)
(SEQ ID NO: 126)
QEEQXKDAXDHANHEXEYQXYQSA,
(vi)
(SEQ ID NO: 132)
ETXDFLXEWFDVXAEDXDYL,
(vii)
(SEQ ID NO: 133)
ETXDFYXEWFDVXAEDXDYL,
(viii)
(SEQ ID NO: 130)
ETXDFFXEWFDVXAEDXDYL,
or
(ix)
(SEQ ID NO: 131)
ETXDFEXEWFDVXAEDXDYL,

wherein each X in the sequences above is an α, α-disubstituted non-natural amino acid with olefinic side chains that can be internally cross-linked, and optionally where each X is (S)-α-(4′-pentenyl)alanine or (R)-α-(4′-pentenyl)alanine. In some instances, each X is (S)-α-(4′-pentenyl)alanine. In some instances, each X is (R)-α-(4′-pentenyl)alanine. These polypeptides can be internally cross-linked or not. In some instances, the polypeptide comprises an internally cross-linked sequence of any one of SEQ ID NOs.:115, 116, 120, 121, 126, or 130-133, optionally wherein the polypeptide is 20 to 25, 20 to 30, 20 to 35, 20 to 40, 20 to 45, 20 to 50, 20 to 55, 20 to 60, 20 to 65, 20 to 70, 20 to 75, 20 to 80, 20 to 90, or 20 to 100 amino acids in length.

In some instances, any of the polypeptides described herein comprise α, α-disubstituted non-natural amino acids with olefinic side chains that can be internally cross-linked. In some instances, the α, α-disubstituted non-natural amino acids with olefinic side chains are inserted at positions that are 2, 3, or 6 amino acids away from one another. In some instances, one of the α, α-disubstituted non-natural amino acids with olefinic side chains is inserted at position 1, 2, 3, 4, 5, 6, 7, 8, 9, 10, 11, 12, 13, 14, 15, 16, 17, 18, 19, or 20 of SEQ ID NO:77 (wherein the position numbering is from the N-terminal E (position 1) to the C-terminal Q (position 20). In some instances, the α, α-disubstituted non-natural amino acids with olefinic side chains are inserted at positions 3 and 7 and/or positions 14 and 15 of SEQ ID NO:77 (wherein the position numbering is from the N-terminal E (position 1) to the C-terminal Q (position 20). In some instances, the α, α-disubstituted non-natural amino acids with olefinic side chains are inserted at positions 3 and 7 and/or positions 13 and 17 of SEQ ID NO:77 (wherein the position numbering is from the N-terminal E (position 1) to the C-terminal Q (position 20). In some instances, the α, α-disubstituted non-natural amino acids with olefinic side chains are inserted at one or more of (i) to (vi) below: (i) positions 5 and 12, (ii) positions 11 and 18, (iii) positions 12 and 19, (iv) positions 14 and 18, (v) positions 15 and 19, or (vi) positions 16 and 20, of SEQ ID NO:77 (wherein the position numbering is from the N-terminal E (position 1) to the C-terminal Q (position 20).

In some instances, the polypeptide is substituted at one or more of positions 2, 3, 4, 5, 6, 7, 8, 9, 10, 11, 12, 13, 17, 18, and 20 of SEQ ID NO:77 (wherein the position numbering is from the N-terminal E (position 1) to the C-terminal Q (position 20). In some instances, the polypeptide is not substituted at one or more of positions 1, 6, 10, 14, 15, 16 and 19 of SEQ ID NO:77 (wherein the position numbering is from the N-terminal E (position 1) to the C-terminal Q (position 20).

In some instances, the polypeptide is 20 to 50 (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids in length. In some instances, the polypeptide includes at least two (e.g., 2, 3, 4, 5, 6, or more) additional amino acids N terminal to position 1 and/or at least two additional amino acids C terminal to position 20 of SEQ ID NO:77.

In some instances, the polypeptide comprises the amino acid sequence:

(i)
(SEQ ID NO: 2)
X₁EEQX₂KTFLDKFNHEAEDLFYQSSXaa₁Xaa₂,
(ii)
(SEQ ID NO: 3)
IEEQX₁KTFX₂DKFNHEAEDLFYQSSXaa₁Xaa₂,
(iii)
(SEQ ID NO: 4)
IEEQAKTX₁LDKX₂NHEAEDLFYQSSXaa₁Xaa₂,
(iv)
(SEQ ID NO: 5)
IEEQAKTFLDKX₁NHEX₂EDLFYQSSXaa₁Xaa₂,
(v)
(SEQ ID NO: 6)
IEEQAKTFLDKFNHEX₁EDLX₂YQSSXaa₁Xaa₂,
(vi)
(SEQ ID NO: 7)
IEEQAKTF8DKFNHEXEDLFYQSSXaa₁Xaa₂,
(vii)
(SEQ ID NO: 8)
IEEQ8KTFLDKXNHEAEDLFYQSSXaa₁Xaa₂,
(viii)
(SEQ ID NO: 9)
IEEQAKTFLDK8NHEAEDXFYQSSXaa₁Xaa₂,
(ix)
(SEQ ID NO: 10)
X₁EEQX₂KTFLDKFNHEX₃EDLX₄YQSSXaa₁Xaa₂,
(x)
(SEQ ID NO: 11)
X₁EEQX₂KTFLDKX₃NHEX₄EDLFYQSSXaa₁Xaa₂,
(xi)
(SEQ ID NO: 12)
IEEQX₁KTFX₂DKFNHEX₃EDLX₄YQSSXaa₁Xaa₂,
(xii)
Xaa₁₁EEQXaa₁₂KXaa₁₃Xaa₁₄Xaa₁₅DKXaa₁₆Xaa₁₇HEXaa₁₈EDXa
a₁₉Xaa₂₀YXa₂₁Xaa₂₂Xaa₂₃Xaa₂₄Xaa₂₅,

wherein
Xaa₁₁=I, A, or a stapling amino acid
Xaa₁₂=A or a stapling amino acid
Xaa₁₃=T, E, or F
Xaa₁₄=F, A, or a stapling amino acid
Xaa₁₅=L, A, or a stapling amino acid
Xaa₁₆=F, A, or a stapling amino acid
Xaa₁₇=N or A
Xaa₁₈=A or a stapling amino acid
Xaa₁₉=L, A, or a stapling amino acid
Xaa₂₀=F, A, or a stapling amino acid
Xaa₂₁=Q or Y
Xaa₂₂=S or A
Xaa₂₃=S or A
Xaa₂₄=L, A, or absent
Xaa₂₅=A or absent (SEQ ID NO:17),
(xiii) Xaa₁₁Xaa₁₂EQXaa₁₃Xaa₁₄TXaa₁₅Xaa₁₆DKXaa₁₇Xaa₁₈HEXaa₁₉Xa a₂₀Xaa₂₁Xaa₂₂Xaa₂₃YQXaa₂₄Xaa₂₅LXaa₂₆, wherein
Xaa₁₁=I, A, or a stapling amino acid
Xaa₁₂=E or A
Xaa₁₃=A or a stapling amino acid
Xaa₁₄=K, R, or A
Xaa₁₅=F, A, or a stapling amino acid
Xaa₁₆=L, A, or a stapling amino acid
Xaa₁₇=F, A, or a stapling amino acid
Xaa₁₈=N or A
Xaa₁₉=A or a stapling amino acid
Xaa₂₀=E, D, or A
Xaa₂₁=D, E, or A
Xaa₂₂=L, A, or a stapling amino acid
Xaa₂₃=F, A, or a stapling amino acid
Xaa₂₄=S or A
Xaa₂₅=S or A
Xaa₂₆=a or absent (SEQ ID NO: 18),
(xiv) IEEQAK6TFLD₁₀K₁₁FNHEAED₁₈LFYQSSLA, wherein K₆ and D₁₀ are linked by a lactam bridge; or wherein K₁₁ and D₁₈ are linked by a lactam bridge (SEQ ID NO: 19),
(xv)
Xaa₁₁EEQXaa₁₂K₆Xaa₁₃Xaa₁₄Xaa₁₅D₁₀K₁₁Xaa₁₆Xaa₁₇HEXaa₁₈ED₁₈
Xaa₁₉Xaa₂₀YXaa₂₁Xaa₂₂Xaa₂₃Xaa₂₄Xaa₂₅, wherein
Xaa₁₁=I or A,
Xaa₁₂=A
Xaa₁₃=T, E, or F
Xaa₁₄=F or A
Xaa₁₅=L or A
Xaa₁₆=F or A
Xaa₁₇=N or A
Xaa₁₈=A
Xaa₁₉=L or A
Xaa₂₀=F or A
Xaa₂₁=Q or Y
Xaa₂₂=S or A
Xaa₂₃=S or A
Xaa₂₄=L or A
Xaa₂₅=A or absent,
wherein K₆ and D₁₀ are linked by a lactam bridge; or wherein K₁₁ and D₁₈ are linked by a lactam bridge (SEQ ID NO: 20),

(xvi)
(SEQ ID NO: 78)
XEEQXKTFLDKFNHEAEDLFYQS,
(xvii)
(SEQ ID NO: 79)
IXEQAXTFLDKFNHEAEDLFYQS,
(xviii)
(SEQ ID NO: 80)
IEXQAKXFLDKFNHEAEDLFYQS,
(xix)
(SEQ ID NO: 81)
IEEQXKTFXDKFNHEAEDLFYQS,
(xx)
(SEQ ID NO: 82)
IEEQAXTFLXKFNHEAEDLFYQS,
(xxi)
(SEQ ID NO: 83)
IEEQAKXFLDXFNHEAEDLFYQS,
(xxii)
(SEQ ID NO: 84)
IEEQAKTXLDKXNHEAEDLFYQS,
(xxiii)
(SEQ ID NO: 85)
IEEQAKTFXDKFXHEAEDLFYQS,
(xxiv)
(SEQ ID NO: 86)
IEEQAKTFLXKFNXEAEDLFYQS,
(xxv)
(SEQ ID NO: 87)
IEEQAKTFLDXFNHXAEDLFYQS,
(xxvi)
(SEQ ID NO: 88)
IEEQAKTFLDKXNHEXEDLFYQS,
(xxvii)
(SEQ ID NO: 89)
IEEQAKTFLDKFXHEAXDLFYQS,
(xxviii)
(SEQ ID NO: 90)
IEEQAKTFLDKFNXEAEXLFYQS,
(xxix)
(SEQ ID NO: 91)
IEEQAKTFLDKFNHXAEDXFYQS,
(xxx)
(SEQ ID NO: 92)
IEEQAKTFLDKFNHEXEDLXYQS,
(xxxi)
(SEQ ID NO: 93)
IEEQAKTFLDKFNHEAXDLFXQS,
(xxxii)
(SEQ ID NO: 94)
IEEQAKTFLDKFNHEAEXLFYXS,
(xxxiii)
(SEQ ID NO: 95)
IEEQAKTFLDKFNHEAEDXFYQX,
(xxxiv)
(SEQ ID NO: 96)
8EEQAKTXLDKFNHEAEDLFYQS,
(xxxv)
(SEQ ID NO: 97)
I8EQAKTFXDKFNHEAEDLFYQS,
(xxxvi)
(SEQ ID NO: 98)
IE8QAKTFLXKFNHEAEDLFYQS,
(xxxvii)
(SEQ ID NO: 99)
IEE8AKTFLDXFNHEAEDLFYQS,
(xxxviii)
(SEQ ID NO: 100)
IEEQ8KTFLDKXNHEAEDLFYQS,
(xxxix)
(SEQ ID NO: 101)
IEEQA8TFLDKFXHEAEDLFYQS,
(xl)
(SEQ ID NO: 102)
IEEQAK8FLDKFNXEAEDLFYQS,
(xli)
(SEQ ID NO: 103)
IEEQAKT8LDKFNHXAEDLFYQS,
(xlii)
(SEQ ID NO: 104)
IEEQAKTF8DKFNHEXEDLFYQS,
(xliii)
(SEQ ID NO: 105)
IEEQAKTFL8KFNHEAXDLFYQS,
(xliv)
(SEQ ID NO: 106)
IEEQAKTFLD8FNHEAEXLFYQS,
(xlv)
(SEQ ID NO: 107)
IEEQAKTFLDK8NHEAEDXFYQS,
(xlvi)
(SEQ ID NO: 108)
IEEQAKTFLDKF8HEAEDLXYQS,
(xlvii)
(SEQ ID NO: 109)
IEEQAKTFLDKFN8EAEDLFXQS,
(xlviii)
(SEQ ID NO: 110)
IEEQAKTFLDKFNH8AEDLFYXS,
or
(xlix)
(SEQ ID NO: 111)
IEEQAKTFLDKFNHE8EDLFYQX,

wherein X₁, X₂, X₃, X₄, X, and 8 are non-natural amino acids with olefinic side chains; Xaa1=L or absent; Xaa2=A or absent. In some instances, the polypeptides of (i)-(xlix) have one or more (e.g., 1, 2, 3, 4, 5, 6, 7) of the following properties:
(i) binds the peptide of SEQ ID NO: 64 or SEQ ID NO: 65;
(ii) inhibits interaction between human ACE2 protein and the S1 protein subunit of SARS-CoV-2;
(iii) inhibits interaction between the carboxypeptidase domain of human ACE2 protein and the S1 protein subunit of SARS-CoV-2;
(iv) inhibits interaction between the carboxypeptidase domain of human ACE2 protein and the RBD of the S1 protein subunit of SARS-CoV-2; (v) competes for human ACE2-SARS-CoV-2 S1 protein subunit binding;
(vi) binds the S1 protein subunit (e.g., the RBD) of a SARS-CoV-2 variant (e.g., one or more of B.1.1.7, B.1.351, P.1, B.1.427, B.1.429, B.1.167); and
(vii) inhibits SARS virus infection, optionally, wherein the SARS infection is SARS-CoV-2 infection.

Also featured herein is a pharmaceutical composition comprising one of the peptides described herein and a pharmaceutically acceptable carrier. In some instances, the pharmaceutical composition includes
(a) means for one or more of:
   (i) binding the peptide of SEQ ID NO: 64 or SEQ ID NO: 65;
   (ii) inhibiting interaction between human ACE2 protein and the S1 protein subunit of SARS-CoV-2;
   (iii) inhibiting interaction between the carboxypeptidase domain of human ACE2 protein and the S1 protein subunit of SARS-CoV-2;
   (iv) inhibiting interaction between the carboxypeptidase domain of human ACE2 protein and the RBD of the S1 protein subunit of SARS-CoV-2;
   (v) inhibiting SARS-CoV-2 virus infection; or
   (vi) binding the S1 protein (e.g., the RBD) of a SARS-CoV-2 variant (e.g., one or more of B.1.1.7, B.1.351, P.1, B.1.427, B.1.429, B.1.167); and
(b) a pharmaceutically acceptable carrier.

Further featured herein are methods of making a stabilized polypeptide. In some instances, the methods include providing any one of the non-crosslinked form of the polypeptides disclosed herein (i.e., any of the polypeptides described herein which comprises an “X” and/or “8” non-natural amino acid and that has not yet been cross-linked) and cross-linking the polypeptide. In some instances, the polypeptide is cross-linked by a ruthenium catalyzed metathesis (RCM) reaction. In some instances, the methods further include formulating the cross-linked polypeptide as a sterile pharmaceutical composition.

Also featured herein is a nanoparticle composition comprising any one of the polypeptides disclosed herein.

Additionally, featured herein is a diagnostic reagent for detecting the presence of a virus whose receptor-binding domain binds to ACE2, the diagnostic reagent comprising a surface comprising one of the polypeptides featured herein. In some instances, the virus is a coronavirus. In some instances, the coronavirus is SARS-CoV-2.

Also featured herein are methods of detecting in a subject the presence of a virus whose receptor-binding domain binds to human ACE2. In some instances, the method include:
(a) providing a detection agent wherein the detection agent is a first polypeptide, wherein the first polypeptide is any of the polypeptides disclosed herein, wherein the first polypeptide binds to the receptor binding domain of the virus, and wherein the first polypeptide is linked to a detection label;
(b) providing a capture agent wherein the capture agent is a second polypeptide, wherein the second polypeptide is any of the polypeptides disclosed herein, wherein the second polypeptide binds to the receptor binding domain of the virus, and wherein the second polypeptide is linked to an affinity label;
(c) mixing a biological sample from the subject with the detection agent and the capture agent to form a mixture;
(d) contacting the mixture with a solid support that binds the capture agent; and
(e) detecting the presence or absence of the virus.

Also included herein are methods for treating or preventing a viral infection caused by a virus that infects cells by binding to ACE2 in a subject, the method comprising administering to a subject in need thereof a therapeutically effective amount of any one of the polypeptides disclosed herein or of any one of the pharmaceutical compositions disclosed herein. In some instances, the viral infection is caused by a coronavirus. In some instances, the coronavirus is HCoV-NL63, SARS-CoV-1, or SARS-CoV-2. In some instances, the coronavirus is SARS-CoV-2.

Also featured herein are methods of treating a subject with post-acute sequelae of SARS-CoV-2 infection, the method comprising administering to the subject a therapeutically effective amount of any one of the polypeptides disclosed herein or of any one of the pharmaceutical compositions disclosed herein. Also featured herein are methods of treating a subject with or at risk of being infected with a variant of SARS-CoV-2, the method comprising administering to the subject a therapeutically effective amount of any one of the polypeptides disclosed herein or of any one of the pharmaceutical compositions disclosed herein. Also featured herein are methods of preventing or inhibiting interaction between the receptor binding domain of a virus and ACE2 in a subject in need thereof, the method comprising administering to the subject a therapeutically effective amount of any one of the polypeptides disclosed herein or of any one of the pharmaceutical compositions disclosed herein.

In some instances of the methods disclosed herein, the subject is a human subject. In some instances, the administering is by nasal drop, nasal spray, nebulization, subcutaneous injection, or intravenous injection.

Also featured herein is a combination therapy for treating or preventing a SARS virus infection, the combination therapy comprising the (a) any one of the polypeptides disclosed herein or of any one of the pharmaceutical compositions disclosed herein and (b) one or more of: dexamethasone, remdesivir, baricitinib in combination with remdesivir, favipiravir, merimepodib, an anticoagulation drug selected from low-dose heparin or enoxaparin, bamlanivimab, a combination of bamlanivimab and etesevimab, a combination of casirivimab and imdevimab, convalescent plasma, an mRNA SARS-CoV-2 vaccine (e.g., those produced by Moderna and Pfizer), an attenuated SARS-CoV-2 virus vaccine, a dead SARS-CoV-2 virus vaccine, a viral vaccine against SARS-CoV-2 (e.g., an adenoviral vaccine such as those produced by Johnson & Johnson or Astrazeneca), an antibody or fragment thereof or small molecule that blocks interaction between hACE2 and the spike protein of SARS-CoV-2, protease inhibitors targeting SARS-CoV-2 S cleavage sites, viral fusion inhibitors (such as, but not limited to, any one or more of the stabilized (stapled/stitched) peptides described in PCT/US2021/020940 (see e.g., FIG. 11 as well as a sequence comprising any one of the sequences of SEQ ID NOs.: 133, 40, 136, 42, 30, 113, 34, 36, 134, 39, 135, 42, 137, 50, 52, 51, 31-33, 37, 41, and 44-49 described therein), which is incorporated by reference herein in its entirety), EK1C4, nelfinavir mesylate, furin inhibitors, or any other agent described in Huang et al., Acta Pharmacologica Sinica (2020) 41:1141-1149; https://doi.org/10.1038/s41401-020-0485-4), or unmethylated CpG dinucleotides in combination with any of these agents.

In some cases the amino acid sequences of any one of SEQ ID NOs: 1, 13-16, 21, 23, 49, 50, 53, 54, 56, or 145-148 are modified to substitute at least two (e.g., 1, 2, 3, 4, 5) amino acids separated by 2, 3, or 6 amino acids with non-natural amino acids with olefinic side chains, which can form an internal cross-link in a ring closing metathesis reaction. In certain cases, each of these peptides (i.e., any one of SEQ ID NOs: 1, 13-16, 21, 23, 49, 50, 53, 54, 56, or 145-148) may also include an N-terminal threonine (T) residue. In some instances, in addition to the substitutions to insert non-natural amino acids with olefinic side chains, the amino acid sequences of SEQ ID NOs: 1, 13-16, 21, 23, 49, 50, 53, 54, 56, or 145-148 has 1 to 16 (i.e., 1, 2, 3, 4, 5, 6, 7, 8, 9, 10, 11, 12, 13, 14, 15, 16) amino acid substitutions. In some cases, the substitutions are on the non-interacting face of the helix (i.e., the part of the helix that does not interact with RBD). In some instances, the structurally-stabilized alpha-helical peptide has 1 to 16 (i.e., 1, 2, 3, 4, 5, 6, 7, 8, 9, 10, 11, 12, 13, 14, 15, 16) amino acid substitutions which are at one or more of positions 1, 2, 5, 6, 8, 9, 12, 13, 16-20, 23, 24, or 26 of SEQ ID NO:1. In some instances, the structurally-stabilized alpha-helical peptide is not substituted at one or more of (e.g., 1, 2, 3, 4, 5, 6, 7, 8, 9, 10) positions 3, 4, 7, 10, 11, 14, 15, 21, 22, or 25. In some instances, the structurally-stabilized alpha-helical peptide is substituted at one or more of (e.g., 1, 2, 3, 4, 5, 6, 7, 8, 9, 10) positions 3, 4, 7, 10, 11, 14, 15, 21, 22, or 25, and the substitution(s) is to a conservative amino acid or alanine.

In one aspect, the disclosure provides a compound comprising a stabilized peptide comprising a sequence having the formula:

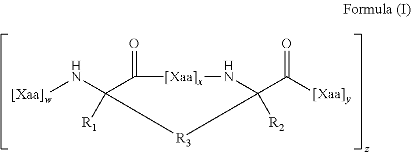

or a pharmaceutically acceptable salt thereof, wherein:
(a) each R₁ and R₂ is independently H, alkyl, alkenyl, alkynyl, arylalkyl, cycloalkylalkyl, heteroarylalkyl, or heterocyclylalkyl, any of which is substituted or unsubstituted;
(b) each R₃ is independently alkylene, alkenylene, or alkynylene, any of which is substituted or unsubstituted;
(c) each x is independently 2, 3, or 6;
(d) each w and y is independently 1, 2, 3, 4, 5, 6, 7, 8, 9, 10, 11, 12, 13, 14, 15, 16, 17, 18, 19, 20, 21, 22, 23, or 24;
(e) z is 1, 2, or 3; and
(f) each Xaa is independently an amino acid.

In some instances, the structurally-stabilized peptide prevents or inhibits the interaction between Angiotensin converting enzyme 2 (ACE2) and a virus whose receptor binding domain binds ACE2. In some instances, the sequence of the compound or the pharmaceutically acceptable salt thereof has the sequence of any one of SEQ ID NO: 1, 21, 77, 112, 113, 117, 118, 123, 125, or 127, or 145-148 with two amino acids separated by 2, 3, or 6 amino acids substituted by α, α-disubstituted non-natural amino acids with olefinic side chains. In some instances, the sequence is SEQ ID NO:1, 21, 77, 112, 113, 117, 118, 123, 125, or 127, or 145-148 with (i) 2, 3, or 4 amino acids substitutions with α, α-disubstituted non-natural amino acids with olefinic side chains that form an internal cross-link; (ii) 0, 1, 2, 3, 4, 5, 6, 7, 8, 9, 10, or 11 additional amino acid substitutions; and/or (iii) 0, 1, 2, 3, 4, or 5 deletions at the N and/or C-terminus of the sequence. In some instances, the sequence includes 3, 2, or 1 deletion at the N-terminus of SEQ ID NO:1. In some instances, the sequence includes 3, 2, or 1 deletion at the C-terminus of SEQ ID NO:1. In some instances, the sequence includes 3, 2, or 1 deletion at the N-terminus of SEQ ID NO:1 and 3, 2, or 1 deletion at the C-terminus of SEQ ID NO:1. These polypeptides have one or more (e.g., 1, 2, 3, 4, 5, 6, 7) of the following properties: (i) binds the peptide of SEQ ID NO: 64 or SEQ ID NO: 65; (ii) inhibits interaction between human ACE2 protein and the S1 protein subunit of SARS-CoV-2; (iii) inhibits interaction between the carboxypeptidase domain of human ACE2 protein and the S1 protein subunit of SARS-CoV-2; (iv) inhibits interaction between the carboxypeptidase domain of human ACE2 protein and the RBD of the S1 protein subunit of SARS-CoV-2; (v) competes for human ACE2-SARS-CoV-2 S1 protein subunit binding; (vi) binds the S1 protein subunit (e.g., the RBD) of a SARS-CoV-2 variant (e.g., one or more of B.1.1.7, B.1.351, P.1, B.1.427, B.1.429, B.1.167); and (vii) inhibits SARS virus infection, optionally, wherein the SARS infection is SARS-CoV-2 infection.

In some instances, the compound or the pharmaceutically acceptable salt thereof comprises one of the following groups of sequences:
(i) [Xaa]_{w} is absent, [Xaa]ₓ is EEQ, and [Xaa]_{y} is KTFLDKFNHEAE(D/Q)LFYQSS (SEQ ID NO: 152) or with 1, 2, 3, 4, 5, 6, 7, 8, 9, or 10 amino acid substitutions;
(ii) [Xaa]_{w} is IEEQ (SEQ ID NO: 24) or with 1 or 2 amino acid substitutions, [Xaa]ₓ is KTF or with 1 or 2 amino acid substitutions, and [Xaa]_{y} is DKFNHEAE(D/Q)LFYQSS (SEQ ID NO: 155) or with 1, 2, 3, 4, 5, or 6 amino acid substitutions;
(iii) [Xaa]_{w} is IEEQAKT (SEQ ID NO: 26) or with 1, 2, 3, or 4 amino acid substitutions, [Xaa]ₓ is LDK or with 1 amino acid substitution, and [Xaa]_{y} is NHEAE(D/Q)LFYQSS (SEQ ID NO: 158) or with 1, 2, 3, 4, or 5 amino acid substitutions;
(iv) [Xaa]_{w} is IEEQAKTFLDK (SEQ ID NO: 29) or with 1, 2, 3, 4, 5, or 6 amino acid substitutions, [Xaa]ₓ is NHE or with 1 amino acid substitution, and [Xaa]_{y} is E(D/Q)LFYQSS (SEQ ID NO: 160) or with 1, 2, 3, 4, or 5 amino acid substitutions;
(v) [Xaa]_{w} is IEEQAKTFLDKFNHE (SEQ ID NO: 32) or with 1, 2, 3, 4, 5, or 6 amino acid substitutions, [Xaa]ₓ is E(D/Q)L or with 1 amino acid substitution, and [Xaa]_{y} is YQSS (SEQ ID NO: 161) or with 1, 2, 3, 4, or 5 amino acid substitutions; (vi) [Xaa]_{w} is IEEQAKTF (SEQ ID NO: 34) or with 1, 2, or 3 amino acid substitutions, [Xaa]ₓ is DKFNHE (SEQ ID NO: 35) or with 1 or 2 amino acid substitutions, and [Xaa]_{y} is E(D/Q)LFYQSS (SEQ ID NO: 160) or with 1, 2, 3, 4, or 5 amino acid substitutions;
(vii) [Xaa]_{w} is IEEQ (SEQ ID NO: 24) or with 1 amino acid substitution, [Xaa]ₓ is KTFLDK (SEQ ID NO: 38) or with 1, 2, or 3 amino acid substitutions, and [Xaa]_{y} is NHEAE(D/Q)LFYQSS (SEQ ID NO: 158) or with 1, 2, 3, 4, 5, or 6 amino acid substitutions;
(viii) [Xaa]_{w} is IEEQAKTFLDK (SEQ ID NO: 29) or with 1, 2, 3, or 4 amino acid substitutions, [Xaa]ₓ is NHEAE(D/Q) (SEQ ID NO: 41) or with 1 amino acid substitution, and [Xaa]_{y} is FYQSS (SEQ ID NO: 163) or with 1, 2, 3, or 4 amino acid substitutions
(ix) [Xaa]_{w} is IEEQ (SEQ ID NO: 24) or with 1, 2, 3, or 4 amino acid substitutions, [Xaa]ₓ is KTA or with 1 amino acid substitution, and [Xaa]_{y} is DKANHEAE(D/Q)AAYQSAXaa₁Xaa₂, wherein Xaa₁ is L, A, or absent, and Xaa₂ is A or absent (SEQ ID NO: 67) or with 1, 2, 3, or 4 amino acid substitutions;
(x) [Xaa]_{w} is IEEQAKT (SEQ ID NO: 26) or with 1, 2, 3, or 4 amino acid substitutions, [Xaa]ₓ is ADK or with 1 amino acid substitution, and [Xaa]_{y} is NHEAEQAAYQSAXaa1Xaa2, wherein Xaa1 is L, A, or absent, and Xaa2 is A or absent (SEQ ID NO: 68) or with 1, 2, 3, or 4 amino acid substitutions; or
(xi) [Xaa]_{w} is IEEQAKTA (SEQ ID NO: 61) or with 1, 2, 3, or 4 amino acid substitutions, [Xaa]ₓ is DKANHE (SEQ ID NO: 62) or with 1 amino acid substitution, and [Xaa]_{y} is EQAAYQSAXaa₁Xaa₂, wherein Xaa1 is L, A, or absent, and Xaa2 is A or absent (SEQ ID NO: 69), wherein Xaa1 is L, A, or absent, and Xaa2 is A or absent or with 1, 2, 3, or 4 amino acid substitutions.

In some instances, any of the above [Xaa]_{y} terminates with the amino acids L and A in that order. In some instances, any of the above [Xaa]_{y} terminates with the amino acid L. In some instances, in any of the above [Xaa]_{w} begins with the amino acid T.

In a second feature, the disclosure provides a compound comprising a stabilized peptide comprising a sequence having the formula:

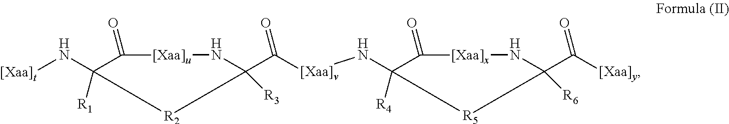

or a pharmaceutically acceptable salt thereof. In some instances, each R₁, R₃, R₄, and R₆ is independently H, alkyl, alkenyl, alkynyl, arylalkyl, cycloalkylalkyl, heteroarylalkyl, or heterocyclylalkyl, any of which is substituted or unsubstituted. In some instances, each R₂ and R₅ is independently alkylene, alkenylene, or alkynylene, any of which is substituted or unsubstituted; optionally wherein R₂ and R₅ are either C₈ alkylene, C₈ alkenylene, or C₈ alkylene; or C₁₁ alkylene, Ci alkenylene, or C₁₁ alkylene. In some instances, each u and x is independently 2, 3, or 6. In some instances, each t, v, and y is independently 1, 2, 3, 4, 5, 6, 7, 8, 9, 10, 11, 12, 13, 14, 15, 16, 17, 18, 19, 20, 21, or 22. In some instances, each Xaa is independently an amino acid. In some instances, the stabilized peptide prevents or inhibits the interaction between Angiotensin converting enzyme 2 (ACE2) and a virus whose receptor binding domain binds ACE2. In some instances, the sequence has SEQ ID NO: 1 with:
(i) at least 4, 5, 6, 7, 8, 9, or 10 amino acids that are substituted with α, α-disubstituted non-natural amino acids with olefinic side chains that form an internal cross-link;
(ii) 0, 1, 2, 3, 4, 5, 6, 7, 8, 9, 10, or 11 additional amino acid substitutions, and
(iii) 0, 1, 2, 3, 4, or 5 amino acid deletions at the N and/or C-terminus of the sequence.

In some instances, the sequence includes 3, 2, or 1 deletion at the N-terminus of SEQ ID NO:1. In some instances, the sequence includes 3, 2, or 1 deletion at the C-terminus of SEQ ID NO:1. In some instances, the sequence includes 3, 2, or 1 deletion at the N-terminus of SEQ ID NO:1 and 3, 2, or 1 deletion at the C-terminus of SEQ ID NO:1.

In some instances, the compound or the pharmaceutically acceptable salt thereof comprises one of the following groups of sequences:
(i) [Xaa]t is absent, [Xaa]u is EEQ, [Xaa]v is KTFLDKFNHE (SEQ ID NO: 43) or with 1, 2, 3, 4, or 5 amino acid substitutions, [Xaa]ₓ is E(D/Q)L or with 1 amino acid substitution, and [Xaa]_{y} is YQSS (SEQ ID NO: 161) or with 1, 2, or 3 amino acid substitutions;
(ii) [Xaa]t is absent, [Xaa]u is EEQ, [Xaa]v is KTFLDK (SEQ ID NO: 38) or with 1, 2, or 3 amino acid substitutions, [Xaa]ₓ is NHE or with 1 amino acid substitution, and [Xaa]_{y} is E(D/Q)LFYQSS (SEQ ID NO: 160) or with 1, 2, 3, 4, or 5 amino acid substitutions; or
(iii) [Xaa]ₜ is IEEQ (SEQ ID NO: 24) or with 1 amino acid substitution, [Xaa]ᵤ is KTF or with 1 or 2 amino acid substitutions, [Xaa]ᵥ is DKFNHE (SEQ ID NO: 48) or with 1 or 2 amino acid substitutions, [Xaa]ₓ is E(D/Q)L or with 1 amino acid substitution, and [Xaa]_{y} is YQSS (SEQ ID NO: 161) or with 1, 2, or 3 amino acid substitutions;
(iv) [Xaa]ₜ is IEEQ (SEQ ID NO: 24) or with 1 amino acid substitution, [Xaa]ᵤ is KTA or with 1 or 2 amino acid substitutions, [Xaa]ᵥ is DKANHE (SEQ ID NO: 165) or with 1 or 2 amino acid substitutions, [Xaa]ₓ is E(D/Q)A or with 1 amino acid substitution, and [Xaa]_{y} is YQSAXaa1Xaa2, wherein Xaa1 is L, A, or absent, and Xaa2 is A or absent (SEQ ID NO: 164) or with 1, 2, or 3 amino acid substitutions;
(v) [Xaa]ₜ is QEEQ (SEQ ID NO: 70) or with 1 amino acid substitution, [Xaa]ᵤ is KDA or with 1 or 2 amino acid substitutions, [Xaa]ᵥ is DHANHE (SEQ ID NO: 165) or with 1 or 2 amino acid substitutions, [Xaa]ₓ is EYQ or with 1 amino acid substitution, and [Xaa]_{y} is YQSAXaa1Xaa2, wherein Xaa1 is L, A, or absent, and Xaa₂ is A or absent (SEQ ID NO: 164) or with 1, 2, or 3 amino acid substitutions;
(vi) [Xaa]ₜ is IEEQ (SEQ ID NO: 24) or with 1 amino acid substitution, [Xaa]ᵤ is KTA or with 1 or 2 amino acid substitutions, [Xaa]ᵥ is DKANHE (SEQ ID NO: 62) or with 1 or 2 amino acid substitutions, [Xaa]ₓ is EQA or with 1 amino acid substitution, and [Xaa]_{y} is YQSAXaa1Xaa2, wherein Xaa₁ is L, A, or absent, and Xaa₂ is A or absent (SEQ ID NO: 44) or with 1, 2, or 3 amino acid substitutions; or
(vii) [Xaa]ₜ is IEEQ (SEQ ID NO: 24) or with 1 amino acid substitution, [Xaa]ᵤ is KEA or with 1 or 2 amino acid substitutions, [Xaa]ᵥ is DKANHE (SEQ ID NO: 62) or with 1 or 2 amino acid substitutions, [Xaa]ₓ is EQA or with 1 amino acid substitution, and [Xaa]_{y} is YQSAXaa1Xaa2, wherein Xaa₁ is L, A, or absent, and Xaa₂ is A or absent (SEQ ID NO: 63) or with 1, 2, or 3 amino acid substitutions.

In some instances, any of the above [Xaa]_{y} terminates with the amino acids L and A in that order. In some instances, any of the above [Xaa]_{y} terminates with the amino acid L. In some instances, in any of the above [Xaa]ₜ begins with the amino acid T.

In a third feature, the present disclosure provides a compound comprising a stabilized peptide comprising a sequence having the formula:

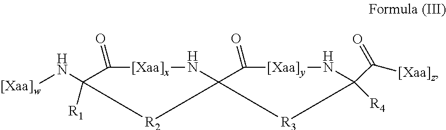

or a pharmaceutically acceptable salt thereof. In some instances, each R₁ and R₄ is independently H, alkyl, alkenyl, alkynyl, arylalkyl, cycloalkylalkyl, heteroarylalkyl, or heterocyclylalkyl, any of which is substituted or unsubstituted. In some instances, each R₂ and R₃ is independently alkylene, alkenylene, or alkynylene, any of which is substituted or unsubstituted. In some instances, each u and x is independently 2, 3, or 6. In some instances, each t, v, and y is independently 1, 2, 3, 4, 5, 6, 7, 8, 9, 10, 11, 12, 13, 14, 15, 16, 17, 18, 19, 20, 21, 22, or 23. In some instances, each Xaa is independently an amino acid. In some instances, the stabilized peptide prevents or inhibits the interaction between Angiotensin converting enzyme 2 (ACE2) and a virus whose receptor binding domain binds ACE2. In some instances, the sequence has SEQ ID NO: 1 with:
(i) 3 amino acids that are substituted with α, α-disubstituted non-natural amino acids with olefinic side chains that form an internal cross-link;
(ii) 0, 1, 2, 3, 4, 5, 6, 7, 8, 9, 10, or 11 additional amino acid substitutions, and
(iii) 0, 1, 2, 3, 4, or 5 amino acid deletions at the N and/or C-terminus of the sequence.
   In some instances, the sequence includes 3, 2, or 1 deletion at the N-terminus of SEQ ID NO:1. In some instances, the sequence includes 3, 2, or 1 deletion at the C-terminus of SEQ ID NO:1. In some instances, the sequence includes 3, 2, or 1 deletion at the N-terminus of SEQ ID NO:1 and 3, 2, or 1 deletion at the C-terminus of SEQ ID NO:1.

In some instances, the sequence is alpha helical, protease resistant, and/or blocks or inhibits infection of human ACE2 expressing epithelial cells of the respiratory system. In some instances, the sequence of the compound or the pharmaceutically acceptable salt thereof has 1 to 16 (e.g., 1, 2, 3, 4, 5, 6, 7, 8, 9, 10, 11, 12, 13, 14, 15, 16) amino acid substitutions which are at one or more of positions 1, 2, 5, 6, 8, 9, 12, 13, 16-20, 23, 24, or 26 of SEQ ID NO:1.

In some instances, if any one or more of positions 1, 8, 9, 12, 13, 19, 20, 23, or 24 of SEQ ID NO:1 are substituted, they are substituted with alanine or a stapling amino acid. In some instances, if position 7 of SEQ ID NO:1 is substituted, it is substituted with glutamic acid or phenylalanine. In some instances, if position 7 of SEQ ID NO:1 is substituted, it is substituted with tyrosine. In some instances, if positions 5 or 16 of SEQ ID NO:1 are substituted, they are substituted with a stapling amino acid. In some instances, one or more (e.g., 1, 2, 3, 4, 5, 6, 7, 8, 9, 10, 11, 12, 13, 14) of positions 1, 2, 6, 8, 9, 12, 13, 17-20, 23, 24, or 26 of SEQ ID NO:1 are substituted to an alanine.

In some instances, the sequence of the compound or the pharmaceutically acceptable salt thereof is any one of SEQ ID NOs:2-12, 134-143, and 172, optionally wherein X, X₁, X₂, X₃, and X₄=(S)-α-(4′-pentenyl)alanine or (R)-α-(4′-pentenyl)alanine, and 8=(R)-α-(7′-octenyl)alanine or (R)-α-(4′-pentenyl)alanine.

In some instances, any one of the following positions are substituted by α, α-disubstituted non-natural amino acids with olefinic side chains: positions 1 and 5 of SEQ ID NO:1; positions 5 and 9 of SEQ ID NO:1; positions 8 and 12 of SEQ ID NO:1; positions 12 and 16 of SEQ ID NO:1; positions 16 and 20 of SEQ ID NO:1; positions 5 and 12 of SEQ ID NO:1; positions 9 and 16 of SEQ ID NO:1; positions 12 and 19 of SEQ ID NO:1; positions 1 and 5 of SEQ ID NO:1 and positions 16 and 20 of SEQ ID NO:1; positions 1 and 5 of SEQ ID NO:1 and positions 12 and 16 of SEQ ID NO:1; or positions and 9 of SEQ ID NO:1 and positions 16 and 20 of SEQ ID NO:1. In some instances, stapling amino acids separated by three amino acids are either (S)-α-(4′-pentenyl)alanine or (R)-α-(4′-pentenyl)alanine, and stapling amino acids separated by six amino acids are (R)-α-(4′-pentenyl)alanine and (S)-α-(7′-octenyl)alanine or (R)-α-(7′-octenyl)alanine and (S)-α-(4′-pentenyl)alanine.

In some instances, when x of Formula (I) is 3; R₃ is C₈ alkylene, C₈ alkenylene, or C₈ alkynylene; and the sum of x, w, and y is at least 10, 11, 12, 13, 14, 15, or 16. In some instances, when x of Formula (I) is 6, R₃ is C₁₁ alkylene, C₁₁ alkenylene, or C₁₁ alkylene, and the sum of x, w, and y is at least 10, 11, 12, 13, 14, 15, or 16.

In some instances, R₃ is substituted with 1, 2, 3, 4, 5, or 6 R₄, and each R₄ is independently —NH₃ or —OH, wherein each —NH₃ is optionally coupled with another chemical entity. In some instances, R₃ is substituted with 2 R₄, and both R₄ are —OH. In some instances, R₃ is substituted with 2 R₄. In some instances, one R₄ is an optionally substituted —NH₃ and the other R₄ is —OH.

In some instances, the pharmaceutically acceptable salt is acetate, sulfate, or chloride.

In some instances, the virus is an alphacoronavirus. In some instances, the virus is a betacoronavirus. In some instances, the virus is HCoV-NL63, SARS-CoV-1, or SARS-CoV-2.

In some instances, the ACE2 is human ACE2 (SEQ ID NO: 22).

Also provided herein is a polypeptide comprising an amino acid sequence of 12 or more (e.g., 12, 13, 14, 15, 16, 17, 18, 19, 20, 21, 22, 23, 24, 25, 26) contiguous amino acids of SEQ ID NO:1, wherein at least two, three, or four amino acids (e.g., 2, 3, 4, 5, 6) of the amino acid sequence separated by 2, 3, or 6 amino acids are substituted by α, α-disubstituted non-natural amino acids with olefinic side chains that can form an internal cross-link; wherein 0 to 14 (1, 2, 3, 4, 5, 6, 7, 8, 9, 10, 11, 12, 13, 14) amino acids of the amino acid sequence are further substituted with another amino acid; and wherein the polypeptide prevents or inhibits the interaction between Angiotensin converting enzyme 2 (ACE2) and a virus whose receptor binding domain binds ACE2. In some instances, one or more (1, 2, 3, 4, 5, 6, 7, 8, 9, 10) of positions 3, 4, 7, 10, 11, 14, 15, 21, 22, or 25 are not substituted. In some instances, if one or more (1, 2, 3, 4, 5, 6, 7, 8, 9, 10) of positions 3, 4, 7, 10, 11, 14, 15, 21, 22, or 25 are substituted, they are substituted with a conservative amino acid or alanine.

Also provided herein is a structurally-stabilized polypeptide comprising an amino acid sequence of 12 or more (e.g., 12, 13, 14, 15, 16, 17, 18, 19, 20, 21, 22, 23, 24, 25, 26) contiguous amino acids of SEQ ID NO:1, wherein at least two, three, or four amino acids (2, 3, 4, 5, 6) of the amino acid sequence separated by 2, 3, or 6 amino acids are substituted by α, α-disubstituted non-natural amino acids with olefinic side chains that form an internal cross-link; wherein 0 to 14 (1, 2, 3, 4, 5, 6, 7, 8, 9, 10, 11, 12, 13, 14) amino acids of the amino acid sequence are further substituted with another amino acid; and wherein the structurally-stabilized polypeptide prevents or inhibits the interaction between Angiotensin converting enzyme 2 (ACE2) and a virus whose receptor binding domain binds ACE2 and has one or more of the properties listed below: (i) is alpha-helical; (ii) is protease resistant; and/or (iii) inhibits infection of an epithelial cell by the virus. In some instances, one or more (1, 2, 3, 4, 5, 6, 7, 8, 9, 10) of positions 3, 4, 7, 10, 11, 14, 15, 21, 22, or 25 are not substituted. In some instances, if one or more (1, 2, 3, 4, 5, 6, 7, 8, 9, 10) of positions 3, 4, 7, 10, 11, 14, 15, 21, 22, or 25 are substituted, they are substituted with a conservative amino acid or alanine. In some instances, the 1 to 14 (1, 2, 3, 4, 5, 6, 7, 8, 9, 10, 11, 12, 13, 14) amino acid substitutions are at one or more of positions 1, 2, 5, 6, 8, 9, 12, 13, 16-20, 23, 24, or 26 of SEQ ID NO:1. In some instances, these substitutions are non-conservative substitutions.

In some instances, the amino acid sequence has 12 to 26 contiguous amino acids of SEQ ID NO:1. In some instances, the amino acid sequence has 1 to 14 (1, 2, 3, 4, 5, 6, 7, 8, 9, 10, 11, 12, 13, 14) amino acid substitutions in addition to the at least two, three, or four (2, 3, 4, 5, 6) amino acids of the amino acid sequence that are substituted by α, α-disubstituted non-natural amino acids with olefinic side chains. In some instances, the polypeptide or the stabilized polypeptide disclosed herein comprises an amino acid sequence set forth in any one of SEQ ID NOs:2-12, 134-143, and 172 except having 1 to 14 (1, 2, 3, 4, 5, 6, 7, 8, 9, 10, 11, 12, 13, 14) amino acid substitutions in SEQ ID NO:2-12, 134-143, and 172, and optionally wherein 8=(R)-α-(7′-octenyl)alanine or (R)-α-(4′-pentenyl)alanine; and X, X₁, X₂, X₃, and X₄=(S)-α-(4′-pentenyl)alanine. In some instances, the 1 to 14 (1, 2, 3, 4, 5, 6, 7, 8, 9, 10, 11, 12, 13, 14) amino acid substitutions are at one or more of positions 1, 2, 5, 6, 8, 9, 12, 13, 16-20, 23, 24, or 26 of SEQ ID NO:1. In some instances, if any one or more of positions 1, 8, 9, 12, 13, 19, 20, 23, or 24 of SEQ ID NO:1 are substituted, they are substituted with alanine or a stapling amino acid. In some instances, if position 7 of SEQ ID NO:1 is substituted, it is substituted with glutamic acid or phenylalanine. In some instances, if position 7 of SEQ ID NO:1 is substituted, it is substituted with tyrosine. In some instances, if positions 5 or 16 of SEQ ID NO:1 are substituted, they are substituted with a stapling amino acid. In some instances, one or more of positions 1, 2, 6, 8, 9, 12, 13, 17-20, 23, 24, or 26 of SEQ ID NO:1 are substituted to an alanine. In some instances, the polypeptide or the stabilized polypeptide disclosed herein comprises an amino acid sequence set forth in any one of SEQ ID NOs:2-12, 134-143, and 172, optionally wherein 8=(R)-α-(7′-octenyl)alanine or (R)-α-(4′-pentenyl)alanine; and X, X₁, X₂, X₃, and X₄=(S)-α-(4′-pentenyl)alanine or (R)-α-(4′-pentenyl)alanine. In some instances, the polypeptide or the stabilized polypeptide disclosed herein comprises the amino acid sequence set forth in SEQ ID NOs:17 or 18.

Also disclosed herein is a stabilized peptide of the peptide with the sequence set forth in any one of SEQ ID NOs: 1, 13, or 14-16 with at least two amino acid substitutions, wherein the at least two amino acid substitutions comprise substitution of amino acids separated by 2, 3, or 6 amino ac ids with a non-natural amino acid with olefinic side chains; optionally further having 1-14 additional amino acid substitutions, and optionally wherein the 1-14 additional amino acid substitutions are at one or more of positions 1, 2, 5, 6, 8, 9, 12, 13, 16-20, 23, 24, or 26 of the sequence.

In some instances, positions 1 and 5 of SEQ ID NO:1; positions 5 and 9 of SEQ ID NO:1; positions 8 and 12 of SEQ ID NO:1; positions 12 and 16 of SEQ ID NO:1; positions 16 and 20 of SEQ ID NO:1; positions 5 and 12 of SEQ ID NO:1; positions 9 and 16 of SEQ ID NO:1; positions 12 and 19 of SEQ ID NO:1; positions 1 and 5 of SEQ ID NO:1 and positions 16 and 20 of SEQ ID NO:1; positions 1 and 5 of SEQ ID NO:1 and positions 12 and 16 of SEQ ID NO:1; or positions 5 and 9 of SEQ ID NO:1 and positions 16 and 20 of SEQ ID NO:1, are substituted by α, α-disubstituted non-natural amino acids with olefinic side chains.

Also provided herein is a stabilized peptide comprising the amino acid sequence set forth in SEQ ID NO:19 or 20, wherein the stabilized peptide prevents or inhibits the interaction between ACE2 and a virus whose receptor binding domain binds ACE2. In some instances, the virus is an alphacoronavirus or betacoronavirus. In some instances, the virus is HCoV-NL63, SARS-CoV-1, or SARS-CoV-2. In some instances, the ACE2 is human ACE2. In some instances, the polypeptide or the stabilized polypeptide is 12 to 50 amino acids in length.

Also provided herein is a pharmaceutical composition comprising the compound or pharmaceutically acceptable salt thereof, or the polypeptide or the stabilized polypeptide as disclosed herein, and a pharmaceutically acceptable carrier.

Also provided herein is a method of treating a coronavirus infection in a subject in need thereof, the method comprising administering to the subject a therapeutically-effective amount of the compound or pharmaceutically acceptable salt thereof, the polypeptide, the stabilized polypeptide, or the pharmaceutical composition disclosed herein.

Also provided herein is a method of preventing a coronavirus infection in a subject in need thereof, the method comprising administering to the subject a therapeutically-effective amount of the compound or pharmaceutically acceptable salt thereof, the polypeptide, the stabilized polypeptide, or the pharmaceutical composition disclosed herein.

In some instances, the coronavirus infection is by an alphacoronavirus or a betacoronavirus that causes infection by binding between its receptor binding domain and ACE2. In some instances, the coronavirus infection is by HCoV-NL63, SARS-CoV-1, or SARS-CoV-2. In some instances, the coronavirus infection is caused by an infection by a SARS-CoV-2 coronavirus.

Also provided herein is a method of preventing or inhibiting interaction between the receptor binding domain of a virus and ACE2 in a subject in need thereof, the method comprising administering to the subject a therapeutically-effective amount of the compound or pharmaceutically acceptable salt thereof, the polypeptide, the stabilized polypeptide, or the pharmaceutical composition disclosed herein.

In some instances, the virus is one that causes infection by binding using its receptor binding domain and ACE2. In some instances, the virus is an alphacoronavirus or a betacoronavirus that causes infection by binding between its receptor binding domain and ACE2. In some instances, the virus is HCoV-NL63, SARS-CoV-1, or SARS-CoV-2. In some instances, the virus is SARS-CoV-2. In some instances, the subject is a human subject. In some instances, the subject is a non-human subject (e.g., a domestic pet or farm animal). In some instances, administering is by nasal drop, nasal spray, nebulization, subcutaneous injection, or intravenous injection.

Also provided herein is a method of making a stabilized polypeptide, the method comprising: (a) providing a polypeptide having the sequence set forth in any one of SEQ ID NOs: 2-12, 17, or 18, 134-143, and 172, and (b) cross-linking the polypeptide.

Also provided herein is a method of making a stabilized polypeptide, the method comprising: (a) providing a polypeptide disclosed herein, and (b) cross-linking the polypeptide. In some instances, the polypeptide is cross-linked by a ruthenium catalyzed metathesis reaction.

Also provided herein is a nanoparticle comprising composition comprising the stabilized peptide disclosed herein In some instances, the compound or pharmaceutically acceptable salt thereof, or the polypeptide or stabilized polypeptide disclosed herein is linked to a detection agent, a moiety that increases half-life, a moiety that improves stability, a moiety that maintains the compound or stabilized polypeptide at the site of infection, or a moiety that improves efficacy. In some instances, the compound or pharmaceutically acceptable salt thereof, or the polypeptide or stabilized polypeptide disclosed herein is linked to polyethylene glycol chitosan, and/or a lipid moiety. In some instances, the compound or pharmaceutically acceptable salt thereof, or the polypeptide or stabilized polypeptide disclosed herein is linked to a detection agent, optionally wherein the detection agent is a fluorophore or a chromophore. In some instances, the compound or pharmaceutically acceptable salt thereof, or the polypeptide or stabilized polypeptide disclosed herein is linked to an affinity tag, optionally wherein the affinity tag is a fusion protein tag, an HA tag, a FLAG tag, a His tag, or a biotin moiety.

Also disclosed herein is a diagnostic for detecting the presence of a virus whose receptor binding domain causes infection by binding to ACE2, the diagnostic comprising a surface comprising a molecule that binds to the receptor binding domain (RBD) of a virus. In some instances, the virus is a coronavirus. In some instances, the surface is a test strip. In some instances, the molecule that binds to the receptor binding domain of a virus is an antibody, a recombinant polypeptide or a stabilized polypeptide, wherein the molecule is immobilized to the surface. In some instances, the virus is HCoV-NL63, SARS-CoV-1, or SARS-CoV-2, optionally wherein the virus is SARS-CoV-2. In some instances, the molecule that binds to the receptor binding domain of a virus is an antibody or antigen-binding fragment that specifically recognizes the RBD of HCoV-NL63, SARS-CoV-1, or SARS-CoV-2. In some instances, the molecule that binds to the receptor binding domain of a virus is a stabilized peptide of SEQ ID NO:1, optionally wherein the stabilized peptide is stapled.

Also disclosed herein is a method of detecting the presence a virus whose receptor binding domain causes infection by binding to ACE2, the method comprising (a) providing a biological sample of a subject; (b) mixing the biological sample a plurality of stabilized peptides disclosed herein to create a mixture, wherein at least one stabilized peptide in the plurality comprises a detection moiety; and at least one stabilized peptide in the plurality comprises a capture moiety; (c) providing a diagnostic disclosed herein; (d) contacting the diagnostic with the mixture; and (e) detecting the presence or absence of the virus.

Also disclosed herein is a method of detecting the presence of a virus whose receptor binding domain causes infection by binding to ACE2 in a subject, the method comprising:
(a) providing a detection agent wherein the detection agent is a stabilized peptide comprising the amino acid sequence of any one of SEQ ID NOs:2-11 with 0 to 11 amino acid substitutions, wherein the first stabilized peptide binds to the receptor binding domain of the virus, and wherein the first stabilized peptide is linked to a detection label; or wherein the detection agent is an antibody or fragment thereof that specifically binds the receptor binding domain of the virus, wherein the antibody or fragment thereof is linked to the detection label;
(b) providing a capture agent wherein the capture agent is a stabilized peptide comprising the amino acid sequence of any one of SEQ ID NOs:2-11 with 0 to 11 amino acid substitutions, wherein the second stabilized peptide binds to the receptor binding domain of the virus, and wherein the second stabilized peptide is linked to an affinity label;
(c) mixing a biological sample from the subject with the detection agent and the capture agent to form a mixture;
(d) contacting the mixture with a solid support that binds the capture agent; and
(e) detecting the presence or absence of the virus.

In some instances, the detection label is a colorimetric molecule, optionally wherein the colorimetric molecule is a chromophore, a fluorophore, horse radish peroxidase, GFP, BFP, CFRP, YFP, or fluorescein, or an affinity moiety such as biotin that can engage a secondary colorimetric molecule such as streptavidin-conjugated HRP. In some instances, the affinity label is biotin, His tag, Flag tag, or Flu tag. In some instances, the solid support comprises streptavidin, Nickel NTA, Flag peptide, or Flu peptide. In some instances, the solid support is a bead, optionally wherein the bead is a magnetic bead. In some instances, the subject is a human subject. In some instances, the biological sample is a nasal sample, a nasopharyngeal sample, an oral sample, a respiratory fluid sample, a lung sample, a blood sample, sputum, mucous, urine or stool. In some instances, the stabilized peptide comprises the amino acid sequence of any one of SEQ ID NOs: 2-12, 17, 18, 134-143, and 172, and wherein the stabilized peptide is attached to a colorimetric label. In some instances, the virus is HCoV-NL63, SARS-CoV-1, or SARS-CoV-2, optionally wherein the virus is SARS-CoV-2. In some instances, the methods disclosed herein further comprise diagnosing the subject with an infection with a virus whose receptor binding domain causes infection by binding to ACE2 when the presence of the virus is detected. In some instances, the methods disclosed herein further comprise administering to a subject who is determined to have the virus with an antiviral therapy, optionally, wherein the antiviral therapy comprises a compound or pharmaceutically acceptable salt thereof, or the stabilized peptide of any disclosed herein. In some instances, the subject is a human.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the disclosure will be apparent from the following detailed description and from the claims.

### BRIEF DESCRIPTION OF DRAWINGS

FIG. 1 shows an enlarged region of the crystal structure of the interaction between the ACE2 receptor (left) and the SARS-CoV-2 receptor binding domain (RBD; right), highlighting the location of the ACE2 helix 1 whose interacting face residues (facing right) are shown to directly engage a surface of the SARS-CoV-2 RBD. The non-interacting face residues of ACE2 helix 1 are facing left.

FIG. 2 shows the first 80 amino acids (SEQ ID NO:66) of human ACE2 (SEQ ID NO:22) compared to Mus musculus (NP_081562.2; SEQ ID NO:167); Rattus norvegicus (NP_001012006.1; SEQ ID NO:168); Danio rerio (XP_005169416.1; SEQ ID NO:169); Sus scrofa (NP_001116542.1; SEQ ID NO:170); Bos taurus (XP_005228485.1; SEQ ID NO:71); Pan troglodytes (XP_016798468.1; SEQ ID NO:72); Macaca mulatta (NP_001129168.1; SEQ ID NO:73); and Canis lupus familiaris (NP_001158732.1; SEQ ID NO:74). The boxed sequences is the ACE2 α1 helix sequence (SEQ ID NO:1).

FIG. 3 shows a non-limiting list of exemplary SAH-ACE2h1 peptides whereby single and double staples are inserted into the ACE2h1 peptide sequence and mutant derivatives thereof.

FIG. 4 shows a variety of non-natural amino acids containing olefinic tethers that can be used to generate hydrocarbon stapled ACE2h1 peptides spanning i, i+3; i, i+4; and i, i+7 positions and single staple scanning to generate a library of singly stapled ACE2h1 peptides.

FIG. 5 shows a variety of staple compositions in multiply stapled peptides and staple scanning to generate a library of multiply stapled ACE2h1 peptides.

FIG. 6 shows a variety of staple compositions in tandem stitched peptides to generate a library of stitched ACE2h1 peptides.

FIG. 7 is an illustration of an exemplary approach to designing, synthesizing, and identifying optimal SAH-ACE2h1 constructs to target the receptor binding domain of SARS-CoV-2 and block its critical interaction with the ACE2 receptor, including the generation of Ala scan, staple scan, and variable N- and C-terminal deletion, addition, and derivatization libraries. Singly and doubly stapled constructs, including alanine and staple scans, are used to identify optimal SAH-ACE2h1 peptides for in vitro and in vivo analyses.

FIG. 8 shows a listing of synthesized SAH-ACE2h1 peptides, including i, i+4 and i, i+7 staple scanning libraries, and a series of single and double stapled analogs bearing point mutations of the ACE2h1 template sequence.

FIGS. 9A-9B show how native and mutated ACE2h1 sequences, when synthesized as a peptide and evaluated by circular dichroism, do not retain the natural alpha-helical structure found in the context of the ACE2 receptor. In contrast, inserting staples at specific locations can restore alpha-helical shape. For the ACE2h1 mutant sequence shown in FIG. 9A, a single staple at a specific location can restore alpha-helical shape, with α-helicity improved even further upon double stapling at the indicated locations. For a distinct ACE2h1 mutant sequence shown in FIG. 9B, installing double staples in the corresponding positions shown in FIG. 9A, converts the random coil conformation of the unstapled sequences into an α-helix.

FIG. 10 shows that insertion of double staples at the indicated positions into ACE2h1 sequences bearing mutations confers striking in vitro proteinase K resistance compared to the unstapled wild-type ACE2h1 sequence.

FIG. 11 shows the mouse plasma stability of an unstapled ACE2h1 mutant sequence and a double-stapled analog. Whereas the unstapled peptide demonstrates a half-life of 374 min, essentially no degradation was observed upon insertion of double staples at the indicated locations.

FIGS. 12A-12B show a bead-binding assay in which His-tagged SARS-CoV-2 receptor binding domain (RBD) protein is bound to Ni-NTA beads and then FITC-ACE2h1 peptides are applied in order to measure binding activity, as monitored by fluorescence imaging of the beads, and thus detection of RBD protein. FIG. 12A shows that an unstapled and mutated ACE2h1 peptide has little to no detectable binding to the RBD-coated beads (FIG. 12A, column A; SEQ ID NO:113), whereas insertion of a single staple at the indicated location (FIG. 12, columns A and B; SEQ ID NOs: 113 and 114, respectively) and then double staples at the indicated locations (FIG. 12A, columns C and E; SEQ ID NOs: 115 and 116, respectively) leads to progressively enhanced binding activity and RBD detection. Importantly, when no RBD protein is added to the beads, the positive fluorescence signal of an exemplary double stapled ACE2h1 peptide seen with RBD-coated beads (FIG. 12A, column C; SEQ ID NO:115) is completely lost (FIG. 12A, column D; SEQ ID NO:115), highlighting the specificity of double stapled peptide binding activity for RBD. FIG. 12B shows that peptide templates bearing a series of mutations can enhance binding to the RBD-coated beads compared to the native sequence (FIG. 12A, columns A-C; SEQ ID NOs: 76, 123, and 125, respectively), including a double stapled analog (FIG. 12A, column D, SEQ ID NO:126).

FIGS. 13A-13C show that the bead-binding results in FIGS. 12A-12B, namely the capacity of a FITC-labeled and stapled ACE2h1 peptide sequence to bind and detect SARS-CoV2 RBD protein, can afford simple and rapid virus detection methods. For example, Peptide A (detection tag) and B (affinity tag) are mixed in a solution (FIG. 13A, part A). The patient sample is added to the peptide solution and mixed (FIG. 13A, part B). The beads are added, mixed, and collected by gravity or centrifugation (FIG. 13A, part C). If the result is negative (no virus present) the peptide containing the chromophore remains in solution (FIG. 13A, part D, top). When the result is positive (virus present in sample), the virus is captured by the beads via peptide B, which is collected along with simultaneous virus-bound peptide A, leading to an immediate read-out (FIG. 13A, part D, bottom). An alternative approach is shown in FIG. 13B and is based on a pregnancy-type strip or ELISA set up in which SAH-ACE2h1 peptide is fixed to a solid support (FIG. 13B, parts A and B), a patient sample is added to the strip or plate well (FIG. 13B, part C), and then application of a second SAH-ACE2h1 peptide is applied (FIG. 13B, part D) that allows for a colorimetric read-out, such as biotinylated SAH-ACE2h1 peptide detected by streptavidin HRP (FIG. 13B, part E) and incubation with chromogenic substrate (FIG. 13B, part F).

FIG. 13C demonstrates the successful development of a SAH-ACE2h1-based test strip, based on this concept of an enzyme-linked stapled peptide assay (ELIPSA), which dose-responsively detects a serial dilution of inactivated SARS-CoV-2 virus (starting titer of 109).

FIGS. 14A-14C show the binding activities of differentially stapled and mutated ACE2h1 peptides for recombinant SARS-CoV-2 proteins containing the RBD. In FIG. 14A, a direct fluorescence polarization binding assay (FPA) is shown in which FITC-labeled double i, i+4 stapled ACE2h1 peptides bearing a series of mutations demonstrate dose-responsive binding activity when incubated with a serial dilution of GST-tagged recombinant SARS-CoV-2 RBD protein. In FIG. 14B, the direct binding interaction between a FITC-labeled double i, i+4 stapled ACE2h1 peptides bearing a series of mutations and the SARS-CoV-2 spike protein is used to screen biotinylated (non-fluorescent) and differentially stapled ACE2h1 peptides, with and without mutations, for competitive spike protein binding activity in solution, revealing constructs that were capable (black bar). In FIG. 14C, a direct fluorescence polarization binding assay (FPA) is shown in which two FITC-labeled double i, i+4 stapled ACE2h1 peptides bearing a series of mutations bound to wild-type SARS-CoV-2 RBD protein and retain at least equivalent, or exhibit more, binding activity to SARS-CoV-2 RBD proteins bearing clinical variants such as the UK (N501Y), or B.1.1.7 lineage, and South African (K₄₁₇N, E484K and N501Y), or B.1.351 lineage, with the latter variant protein in particular showing enhanced binding activity to both SAH-ACE2h1 peptides.

FIGS. 15A-15B show an alternative binding analysis in which biotinylated stapled ACE2h1 peptides were applied to a streptavidin-coated tip (solid support) and tested for recombinant SARS-CoV-2 RBD binding activity by biolayer interferometry (BLI). In FIG. 15A, single i, i+4 or single i, i+7 stapled ACE2h1 peptides were tested for SARS-CoV-2 RBD binding activity at a screening dose, revealing compounds that do or do not bind to RBD based on peptide sequence, staple type, and/or staple position. In FIG. 15B, double i, i+4 stapled ACE2h1 peptides bearing a series of mutations show dose-responsive RBD binding activity.

FIGS. 16A-16B show a SARS-CoV-2 pseudovirus assay in which a virus coated with the SARS-CoV-2 Spike protein containing RNA coding for GFP is used to infect 293T cells that are treated with ACE2h1 peptides, followed by fluorescence microscopy imaging to detect and compare the infection levels between vehicle and peptide treatments to assay for inhibition of viral infection (FIG. 16A), which was quantitated by image analysis (FIG. 16B). Insertion of a single staple conferred antiviral activity compared to the unstapled template peptide that showed no activity, with double stapling enhancing antiviral activity further. Installing a series of mutations into the template sequence or double stapling an N- and C-terminally truncated construct bearing a series of distinct mutations also conferred marked antiviral activity in the pseudovirus assay.

FIGS. 17A-17C show the comparative inhibitory effects of differentially stapled and mutated ACE2h1 peptides on Vero E6 cell infection by native SARS-CoV-2 virus, which is detected and quantitated by a high throughput immunofluorescence assay.

FIG. 17A shows how peptides are initially screened for inhibitory activity at a 25 μM test dose, which is followed by evaluating hits by 2-fold serial dilution from a 25 μM starting dose, as shown in FIG. 17B and FIG. 17C, revealing differentially stapled and mutated ACE2h1 peptides with dose-responsive antiviral activity.

FIGS. 18A-18C shows how lead SAH-ACE2h1 peptides are identified based on consistency of activity across a diversity of assays, including binding assays with peptides in solution or on solid support and antiviral assays using SARS-CoV-2 pseudovirus or native virus. FIG. 18A shows an exemplary single i, i+4 stapled peptide of the native sequence with no biological activity across 4 assays and 5 single i, i+4 stapled peptides of the native sequence that demonstrated biological activity in 3 of 4 functional assays. FIG. 18B shows two exemplary i, i+7 single stapled peptides of the native sequence with no biological activity across 4 assays and 8 single i, i+7 single stapled peptides of the native sequence that demonstrated biological activity in at least 3 of 4 functional assays, with 3 compositions demonstrating efficacy across all RBD binding and antiviral assays. FIG. 18C shows how favorable staple types and locations identified by staple scanning and incorporated into templates iterated by amino acid mutagenesis can lead to a series of lead constructs with consistent biological activity across 4 independent assays, spanning soluble binding, solid phase binding, pseudovirus infectivity, and native virus infectivity assays.

### DETAILED DESCRIPTION

Coronavirus disease 2019 is an infectious disease that has spread across the world. It is caused by a novel coronavirus, severe acute respiratory syndrome coronavirus 2 (SARS-CoV-2). The spike (S) protein of SARS-CoV-2, which plays a key role in the receptor recognition and cell membrane fusion process, is composed of two subunits, S1 and S2. The S1 subunit contains a receptor-binding domain that recognizes and binds to the host receptor angiotensin-converting enzyme 2, while the S2 subunit mediates viral cell membrane fusion by forming a six-helical bundle via the two-heptad repeat domain. This disclosure relates, in part, to antiviral peptides targeting the S protein.

The present disclosure features structurally-stabilized (e.g., stapled, stitched) peptides of the ACE2 α1 helix that can block or inhibit binding by one or more coronaviruses (e.g., betacoronaviruses such as SARS-CoV-2) to a cell (e.g., a human respiratory epithelial cell). In the case of SARS-CoV-1 and SARS-CoV-2, the RBD in the spike glycoprotein (i.e., S1 subunit) of the coronavirus interacts with the al helix of ACE2. The stabilized peptides of this disclosure inhibit or block the interaction between ACE2 on a cell with the receptor binding domain (RBD) of a virus that binds ACE2, such as a coronavirus (e.g., SARS-CoV-2). Accordingly, the present disclosure provides novel methods and compositions (e.g., combinations of compositions) for treating, for developing treatments for, for preventing infection with, and for diagnosing infection by one or more coronaviruses (e.g., betacoronaviruses such as SARS-CoV-2).

### Angiotensin I Converting Enzyme 2 (ACE2)

The mechanism for SARS-CoV-2 infection is the requisite binding of the virus to the membrane-bound form of angiotensin-converting enzyme 2 (ACE2) and internalization of the complex by the host cell. Functionally, there is an interaction between ACE2 α1 helix and the S1 protein of the SARS-CoV-2 virus. S1 contains the receptor binding domain (RBD), which directly binds to the peptidase domain (PD) of angiotensin-converting enzyme 2 (ACE2). Li et al., Science 309, 1864-1868 (2005).

The ACE2 domain involved in the interaction with RBD is called the carboxypeptidase domain and encompasses residues 1-612 of human ACE 2 protein. The ACE2 α1 helix sequence spans from approximately position 20 to position 54 of the human ACE2 protein. An exemplary sequence for ACE2 α1 helix sequence that engages the RBD of SARS-CoV-2 (e.g. amino acids 21 to 46) is provided as IEEQAKTFLDKFNHEAEDLFYQSSLA (SEQ ID NO:1). The amino acid sequence of an exemplary human angiotensin I converting enzyme 2 (ACE2) helix 1 sequence that interacts with the RBD is provided as SEQ ID NO:22, shown below.

(SEQ ID NO: 22)
MSSSSWLLLS LVAVTAAQST IEEQAKTFLD KENHEAEDLE
YQSSLASWNY NTNITEENVQ NMNNAGDKWS AFLKEQSTLA
QMYPLQEIQN LTVKLQLQAL QQNGSSVLSE DKSKRLNTIL
NTMSTIYSTG KVCNPDNPQE CLLLEPGLNE IMANSLDYNE
RLWAWESWRS EVGKQLRPLY EEYVVLKNEM ARANHYEDYG
DYWRGDYEVN GVDGYDYSRG QLIEDVEHTF EEIKPLYEHL
HAYVRAKLMN AYPSYISPIG CLPAHLLGDM WGREWTNLYS
LTVPFGQKPN IDVTDAMVDQ AWDAQRIFKE AEKFFVSVGL
PNMTQGFWEN SMLTDPGNVQ KAVCHPTAWD LGKGDFRILM
CTKVTMDDEL TAHHEMGHIQ YDMAYAAQPF LLRNGANEGE
HEAVGEIMSL SAATPKHLKS IGLLSPDFQE DNETEINELL
KQALTIVGTL PFTYMLEKWR WMVFKGEIPK DQWMKKWWEM
KREIVGVVEP VPHDETYCDP ASLFHVSNDY SFIRYYTRTL
YQFQFQEALC QAAKHEGPLH KCDISNSTEA GQKLENMLRL
GKSEPWTLAL ENVVGAKNMN VRPLLNYFEP LFTWLKDQNK
NSFVGWSTDW SPYADQSIKV RISLKSALGD KAYEWNDNEM
YLERSSVAYA MRQYFLKVKN QMILFGEEDV RVANLKPRIS
ENFFVTAPKN VSDIIPRTEV EKAIRMSRSR INDAFRLNDN
SLEFLGIQPT LGPPNQPPVS IWLIVFGVVM GVIVVGIVIL
IFTGIRDRKK KNKARSGENP YASIDISKGE NNPGFQNTDD
VQTSF

Underlined in SEQ ID NO:22 is the ACE2 α1 helix sequence that spans from position 21 to position 46 (i.e., SEQ ID NO:1). In some instances, the ACE2 α1 helix sequence targets by the Receptor Binding Domain (RBD) and Receptor Binding Motif (RBM) of SARS-CoV-2 and other ACE2 interacting viruses (e.g., SARS-CoV-1, HCoV-NL63). In some instances, the ACE2 α1 helix sequence targets a variant of SARS-CoV-2. Variants are disclosed in Peacock et al., Journal of General Virology, (2021); 102:001584, which is incorporated by reference in its entirety. In some instances, the RBD amino acid sequence has the amino acid sequence of SEQ ID NO:64.

| (SEQ ID NO: 64) | |
|---|---|
| 1 | rvqptesivr fpnitnlcpf gevfnatrfa svyawnrkri sncvadysvl ynsasfstfk |
| 61 | cygvsptkln dlcftnvyad sfvirgdevr qiapgqtgki adynyklpdd ftgcviawns |
| 121 | nnldskvggn ynylyrlfrk snlkpferdi steiyqagst pcngvegfnc yfplqsygfq |
| 181 | ptngvgyqpy rvvvlsfell hapatvcgpk kstnlvknkc vnf |

In some instances, the RBD is an ACE2 binding fragment of the amino acid sequence of SEQ ID NO:64. In one embodiment the ACE2 binding fragment of RBD is

| (SEQ ID NO: 65) | |
|---|---|
| | fpnitnlcpf gevfnatrfa svyawnrkri sncvadysvl ynsasfstfk |
| 61 | cygvsptkln dlcftnvyad sfvirgdevr qiapgqtgki adynyklpdd ftgcviawns |
| 121 | nnldskvggn ynylyrlfrk snlkpferdi steiyqagst pcngvegfnc yfplqsygfq |
| 181 | ptngvgyqpy rvvvlsfell. |

In certain instances, each of the peptides and stabilized peptides described herein can bind SEQ ID NO:64 or 65.

Exemplary amino acid sequence of the ACE2 α1 helix sequence and variants thereof are provided in Table 1. Note that each of these amino acid sequences may further include an N-terminal threonine (T) amino acid residue. All such variants and their use in the methods described herein are also encompassed by this disclosure.

TABLE 1
ACE2 Helix 1 Peptide Analogs.

| Name | Sequence | SEQ ID NO: |
|---|---|---|
| ACE2h1 | IEEQAKTFLDKFNHEAEDLFYQSSLA | 1 |
| (native)-26 | | |
| ACE2h1 | IEEQAKTFLDKFNHEAEDLFYQSS | 21 |
| (native)-24 | | |
| ACE2h1 | IEEQAKTFLDKFNHEAEDLFYQS | 76 |
| (native)-23 | | |
| (21-43) | | |
| ACE2h1 | EQAKTFLDKFNHEAEDLFYQ | 77 |
| (native)-20 | | |
| (23-42) | | |
| SAH- | Xₐₐ₁EEQAKTXₐₐ₂ Xₐₐ₃DKXₐₐ₄ Xₐₐ₅HEAED Xₐₐ₆ | 13 |
| ACE2h1- | Xₐₐ₇YQ Xₐₐ₈ Xₐₐ₉ Xₐₐ₁₀ Xₐₐ₁₁, wherein | |
| 12 | Xₐₐ₁ = I or A | |
| | Xₐₐ₂ = F or A | |
| | Xₐₐ₃ = L or A | |
| | Xₐₐ₄ = F or A | |
| | Xₐₐ₅ = N or A | |
| | Xₐₐ₆ = L or A | |
| | Xₐₐ₇ = F or A | |
| | Xₐₐ₈ = S or A | |
| | Xₐₐ₉ = S or A | |
| | Xₐₐ₁₀ = L, A, or absent | |
| | Xₐₐ₁₁ = A or absent | |
| SAH- | IEEQAKTFLDKFNHEAEDLFYXₐₐ₁SSXₐₐ₂ Xₐₐ₃, | 14 |
| ACE2h1- | wherein | |
| 13 | Xₐₐ₁ = Q or Y | |
| | Xₐₐ₂ = L, A, or absent | |
| | Xₐₐ₃ = A or absent | |
| SAH- | IEEQAKXₐₐ₁FLDKFNHEAEDLFYQSSXₐₐ₂ Xₐₐ₃, | 15 |
| ACE2h1- | wherein | |
| 14 | Xₐₐ₁ = T or E | |
| | Xₐₐ₂ = L, A, or absent | |
| | Xₐₐ₃ = A or absent | |
| SAH- | IEEQAKXₐₐ₁FLDKFNHEAEDLFYQSSXₐₐ₂ Xₐₐ₃, | 16 |
| ACE2h1- | wherein | |
| 15 | Xₐₐ₁ = T or F | |
| | Xₐₐ₂ = L, A, or absent | |
| | Xₐₐ₃ = A or absent | |
| SAH- | Xₐₐ₁EEQAKTXₐₐ₂ Xₐₐ₃DKXₐₐ₄ Xₐₐ₅HEAE(D/Q) | 145 |
| ACE2h1- | Xₐₐ₆ Xₐₐ₇YQ Xₐₐ₈ Xₐₐ₉, wherein | |
| 12-alt | Xₐₐ₁ = I or A | |
| | Xₐₐ₂ = F or A | |
| | Xₐₐ₃ = L or A | |
| | Xₐₐ₄ = F or A | |
| | Xₐₐ₅ = N or A | |
| | Xₐₐ₆ = L or A | |
| | Xₐₐ₇ = F or A | |
| | Xₐₐ₈ = S or A | |
| | Xₐₐ₉ = S or A | |
| SAH- | Xaa1EEQAKTXaa2 Xaa3DKXaa4 Xaa5HEAE(D/Q) | 145 |
| ACE2h1- | Xaa6 Xaa7YQ Xaa8 Xaa9, wherein | |
| 12-alt | | |
| SAH- | IEEQAKTFLDKFNHEAE(D/Q)LFYX′ₐₐ₁SS, | 146 |
| ACE2h1- | wherein Xₐₐ₁ = Q or Y | |
| 13-alt | | |
| SAH- | IEEQAKX′ₐₐ₁FLDKFNHEAE(D/Q)LFYQSS, | 147 |
| ACE2h1- | wherein X′ₐₐ₁ = T or E | |
| 14-alt | | |
| SAH- | IEEQAKX′ₐₐ₁FLDKFNHEAE(D/Q)LFYQSS, | 148 |
| ACE2h1- | wherein X′ₐₐ₁ = T or F | |
| 15-alt | | |
| Native | IEEQAKTFLDKFNHEAEDLFYQSXaa₁Xaa₂Xaa₃, | 49 |
| ACE2h1 | wherein Xaa₁ is A or absent, Xaa₂ is L, A, or absent, | |
| peptide | Xaa₂ is A or absent, and Xaa₃ is A or absent | |
| SAH- | IEEQAKTAADKANHEAEDAAYQSAXaa₁Xaa₂, | 50 |
| ACE2h1- | wherein Xaa₁ is L, A, or absent, and Xaa₂ is A or absent | |
| mut-1 | | |
| SAH- | IQEQAKTDADKHNHEAEDYQYQSAXaa₁Xaa₂, | 53 |
| ACE2h1- | wherein Xaa₁ is L, A, or absent, and Xaa₂ is A or absent | |
| mut-4 | | |
| SAH- | QEEQAKDAADHANHEAEYQAYQSAXaa₁Xₐa₂, | 54 |
| ACE2h1- | wherein Xaa₁ is L, A, or absent, and Xaa₂ is A or absent | |
| mut-5 | | |
| SAH- | IEEQAKTAADKANHEAEQAAYQSAXaa₁Xaa₂, | 56 |
| ACE2h1- | wherein Xaa₁ is L, A, or absent, and Xaa₂ is A or absent | |
| mut-7 | | |

In certain instances, the ACE2 α1 helix sequence peptides described herein (e.g., SEQ ID NOs: 1, 13-16, 21, 49, 50, 53, 54, 56, 76, 77, 112, 113, 117, 118, 123, 125, 127, or 145-148 may also contain one or more (e.g., 1, 2, 3, 4, 5, 6, 7, 8, 9, 10, 11, 12, 13, 14, 15, 16) amino acid substitutions. These substitutions may be conservative and/or non-conservative amino acid substitutions. In addition, in some instances at least two (e.g., 2, 3, 4, 5, or 6) amino acids of SEQ ID NOs: 1, 13-16, 21, 49, 50, 53, 54, 56, 76, 77, 112, 113, 117, 118, 123, 125, or 127, or 145-148 may be substituted by α, α-disubstituted non-natural amino acids with olefinic side chains.

In some instances, any of the peptides in Table 1 (SEQ ID NO: 1, 13-16, 21, 49, 50, 53, 54, 56, or 145-148) may include Xaa₁ (wherein Xaa₁ is L or absent), or Xaa₂ (wherein Xaa1 is A, or absent). In some instances, Xaa1, and Xaa₂ are absent, in any of SEQ ID NO: 1, 13-16, 21, 49-56, or 145-148 and these peptides include an N-terminal threonine (T). In some instances, when both Xaa1, and Xaa₂ are both present in any of SEQ ID NOs: 1, 13-16, 21, 49-56, 76, 77, or 145-148 these peptides can further include an N-terminal threonine (T).

In some instances, the disclosed polypeptides comprises EQAKTFLDKFNHEAEDLFYQ (SEQ ID NO:77). In some instances, the polypeptides comprises an amino acid sequence that is at least 30%, and less than 81% identical to, or that has at least 4 and up to 14 amino acid substitutions in, (SEQ ID NO:77). In some instances, the polypeptide comprises an amino acid sequence that is at least 30%, at least 35%, at least 40%, at least 45%, at least 50%, at least 55%, at least 60%, at least 65%, at least 70%, at least 71%, at least 75% or at least 81% identical to SEQ ID NO:77. In some instances, the polypeptides comprises an amino acid sequence that is at less than 61%, 71%, or 81% identical to SEQ ID NO:77. In some instances, the polypeptide comprises an amino acid sequence that has at least 4, at least 5, at least 6, at least 7, at least 8, at least 9, at least 10, at least 11, at least 12, at least 13, or at least 14 amino acid substitutions compared to SEQ ID NO:77. As disclosed herein, substitutions include changes from an amino acid of SEQ ID NO:77 to a naturally-occurring amino acid or to a stabilized amino acid (e.g., comprising a stitch).

In some instances, the polypeptide comprises SEQ ID NO:77 and is substituted at one or more (e.g., 1, 2, 3, 4, 5, 6, 7, 8, 9, 10, 11, 12, 13, 14, or 15) of positions 2, 3, 4, 5, 6, 7, 8, 9, 10, 11, 12, 13, 17, 18, and 20 of SEQ ID NO:77 (wherein the position numbering is from the N-terminal E (position 1) to the C-terminal Q (position 20). In some instances, the polypeptide comprises SEQ ID NO:77 and is substituted at position 2 (Q). In some instances, the polypeptide comprises SEQ ID NO:77 and is substituted at position 3 (A). In some instances, the polypeptide comprises SEQ ID NO:77 and is substituted at position 4 (K). In some instances, the polypeptide comprises SEQ ID NO:77 and is substituted at position 5 (T). In some instances, the polypeptide comprises SEQ ID NO:77 and is substituted at position 6 (F). In some instances, the polypeptide comprises SEQ ID NO:77 and is substituted at position 7 (L). In some instances, the polypeptide comprises SEQ ID NO:77 and is substituted at position 8 (D). In some instances, the polypeptide comprises SEQ ID NO:77 and is substituted at position 9 (K). In some instances, the polypeptide comprises SEQ ID NO:77 and is substituted at position 10 (F). In some instances, the polypeptide comprises SEQ ID NO:77 and is substituted at position 11 (N). In some instances, the polypeptide comprises SEQ ID NO:77 and is substituted at position 12 (H). In some instances, the polypeptide comprises SEQ ID NO:77 and is substituted at position 13 (E). In some instances, the polypeptide comprises SEQ ID NO:77 and is substituted at position 17 (L). In some instances, the polypeptide comprises SEQ ID NO:77 and is substituted at position 18 (F). In some instances, the polypeptide comprises SEQ ID NO:77 and is substituted at position 20 (Q). The polypeptide has one or more of the following properties: (i) binds the peptide of SEQ ID NO: 64 or SEQ ID NO: 65; (ii) inhibits interaction between human ACE2 protein and the S1 protein subunit of SARS-CoV-2; (iii) inhibits interaction between the carboxypeptidase domain of human ACE2 protein and the S1 protein subunit of SARS-CoV-2; (iv) inhibits interaction between the carboxypeptidase domain of human ACE2 protein and the RBD of the S1 protein subunit of SARS-CoV-2; (v) competes for human ACE2-SARS-CoV-2 S1 protein subunit binding; (vi) binds the S1 protein subunit (e.g., the RBD) of a SARS-CoV-2 variant; and (vii) inhibits SARS virus infection, optionally, wherein the SARS infection is SARS-CoV-2 infection.

A “conservative amino acid substitution” means that the substitution replaces one amino acid with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), and acidic side chains and their amides (e.g., aspartic acid, glutamic acid, asparagine, glutamine).

In some instances, the ACE2 α1 helix sequence peptides described herein (e.g., SEQ ID NO: 1, 13-16, 21, 49, 50, 53, 54, 56, 76, 77, 112, 113, 117, 118, 123, 125, 127, or 145-148) may also contain at least one, at least 2, at least 3, at least 4, or at least 5 amino acids added to or deleted from the N-terminus of the peptide. In one instance any one of the peptides of SEQ ID NO: 1, 13-16, 21, 49, 50, 53, 54, 56, 76, 77, 112, 113, 117, 118, 123, 125, 127, or 145-148 has an added threonine (T) at the N-terminus of the sequence. In some instances, the ACE2 α1 helix sequence peptides described herein (e.g., SEQ ID NO: 1, 13-16, or 21, 49, 50, 53, 54, 56, 76, 77, 112, 113, 117, 118, 123, 125, 127, or 145-148) may also contain at least one, at least 2, at least 3, at least 4, or at least 5 amino acids added to or deleted from the C-terminus of the peptide.

In some instances, the amino acids of the interacting face of SEQ ID NO:1 are at positions 3 (corresponding to E23 of SEQ ID NO:22), 4 (Q24), 7 (T27), 10 (D30), 11 (K31), 14 (H34), 15 (E35), 21 (Y41), 22 (Q42), and 25 (L45). In some cases, the amino acids of the interacting face are not substituted. In some cases 1 to 10, 1 to 9, 1 to 8, 1 to 7, 1 to 6, 1 to 5, 1 to 4, 1 to 3, 1 to 2, or 1 amino acids of the interacting face are substituted (e.g., conservative amino acid substitutions). In some cases 1 to 10, 1 to 9, 1 to 8, 1 to 7, 1 to 6, 1 to 5, 1 to 4, 1 to 3, 1 to 2, or 1 amino acids of the interacting face are substituted to alanine. In some instances, the amino acids of the non-interacting face of SEQ ID NO:1 are at positions 1 (corresponding to 121 of SEQ ID NO:22), 2 (E22), 5 (A25), 6 (K26), 8 (F28), 9 (L29), 12 (F32), 13 (N33), 16 (A36), 17 (E37), 18 (D38), 19 (L39), 20 (F40), 23 (S43), 24 (S44), and 26 (A46). In some cases, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 16, 15, 14, 13, 12, 11, 10, 9, 8, 7, 6, 5, 4, 3, 2, or 1 amino acids of the non-interacting face are substituted (e.g., non-conservative and/or conservative amino acid substitutions). In some cases, the amino acids of the non-interacting face at one or more of positions 2, 5, 8, 9, 12, and 13 of SEQ ID NO:1 are substituted by non-conservative and/or conservative amino acid substitutions. In some cases, the amino acids of the non-interacting face at one or more of positions 1, 2, 8, 9, 12, 13, 19, 20, 23, or 24 of SEQ ID NO:1 are substituted by non-conservative and/or conservative amino acid substitutions. In some cases, 1 to 7, 1 to 6, 1 to 5, 1 to 4, 1 to 3, 1 to 2, or 1 of the amino acids of the non-interacting face are substituted. In some cases the substitutions are non-conservative amino acid substitutions. In other cases, the substitutions are conservative amino acid substitutions. In some cases, the substitutions include both conservative and non-conservative amino acid substitutions.

In some instances, substitutions can be made at one or more (e.g., 1, 2, 3, 4, 5, 6, 7, 8, 9, 10, 11, 12, 13, 14, 15, 16) of positions 1, 2, 4-20, 22-24, 25, and 26, wherein the position numbering is provided with respect to SEQ ID NO:1. In some instances, α,α-disubstituted non natural amino acids can be introduced in to the polypeptide (to enable internal cross-linking) at one or more of the following groups of positions: 5 and 9; 15 and 19; 16 and 20; 14 and 18; 18 and 22; 19 and 23; 9 and 16; 10 and 17; 11 and 18; 12 and 19; 13 and 70; or 15 and 22, wherein the position numbering is provided with respect to SEQ ID NO:1.

In some instances, each peptide in Table 1 can include beta alanine at the N-terminus. In some embodiments, a conjugate can be coupled to the N-terminus of the peptides in Table 1. In some instances, the conjugate is a detection moiety disclosed herein (e.g., FITC (fluorescein isothiocyanate)). In some instances, the conjugate is a capture moiety disclosed herein (e.g., a biotin moiety).

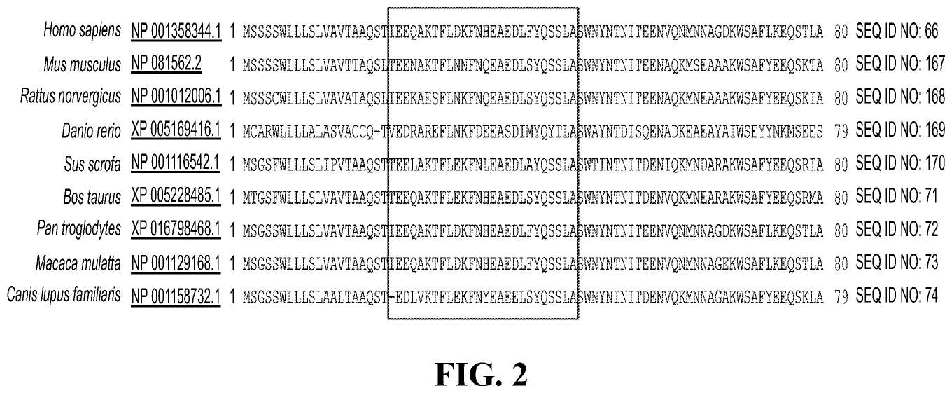

Further exemplary amino acid sequences of the ACE2 in Mus musculus (NP_081562.2); Rattus norvegicus (NP_001012006.1); Danio rerio (XP_005169416.1); Sus scrofa (NP_001116542.1); Bos taurus (XP_005228485.1); Pan troglodytes (XP_016798468.1); Macaca mulatta (NP_001129168.1); and Canis lupusfamiliaris (NP_001158732.1) are aligned to the first 80 amino acids of SEQ ID NO:22, as shown in FIG. 2, with the ACE2 α1 helix sequence boxed. In some instances, IEEQAKTFLDKFNHEAEDLFYQSSLA (SEQ ID NO:1) varies at different amino acid positions based on differences in amino acid sequences provided in FIG. 2 and listed in Table 2.

TABLE 2
ACE2 Helix 1 Peptide Analogs.

| Name | Sequence | SEQ ID NO: |
|---|---|---|
| ACE2h1 | IEEQAKTFLDKFNHEAEDLFYQSSLA | 1 |
| (native) | | |
| ACE2h1 | Xaa₁EXaa₂Xaa₃Xaa₄Xaa₅Xaa₆FLXaa₇Xaa₈FXaa₉ | 151 |
| homologous | Xaa₁₀EAXaa₁₁Xaa₁₂Xaa₁₃Xaa₁₄YQXaa₁₅Xaa₁₆SS | |
| variant | LA, wherein | |
| | Xaa₁ = I, T, V, or is absent | |
| | Xaa₂ = E or D | |
| | Xaa₃ = Q, N, K, R, or L | |
| | Xaa₄ = A or V | |
| | Xaa₅ = K, E, or R | |
| | Xaa₆ = T, S, or E | |
| | Xaa₇ = D, N, or E | |
| | Xaa₈ = K or N | |
| | Xaa₉ = N or D | |
| | Xaa₁₀ = H, Q, E, L, or Y | |
| | Xaa₁₁ = E or S | |
| | Xaa₁₂ = D or E | |
| | Xaa₁₃ = L or I | |
| | Xaa₁₄ = F, S, M, Nle (B), or A | |
| | Xaa₁₅ = S or Y | |
| | Xaa₁₆ = S or T | |

In some instances, each peptide in Table 2 can include beta alanine at the N-terminus. In some embodiments, a conjugate can be coupled to the N-terminus of the peptides listed in Table 2. In some instances, the conjugate is a detection moiety disclosed herein (e.g., FITC (fluorescein isothiocyanate)). In some instances, the conjugate is a capture moiety disclosed herein (e.g., a biotin moiety).

The above described polypeptides have one or more (e.g., 1, 2, 3, 4, 5, 6, 7) of the following properties: (i) binds the peptide of SEQ ID NO: 64 or SEQ ID NO: 65; (ii) inhibits interaction between human ACE2 protein and the S1 protein subunit of SARS-CoV-2; (iii) inhibits interaction between the carboxypeptidase domain of human ACE2 protein and the S1 protein subunit of SARS-CoV-2; (iv) inhibits interaction between the carboxypeptidase domain of human ACE2 protein and the RBD of the S1 protein subunit of SARS-CoV-2; (v) competes for human ACE2-SARS-CoV-2 S1 protein subunit binding; (vi) binds the S1 protein subunit (e.g., the RBD) of a SARS-CoV-2 variant (e.g., one or more of B.1.1.7, B.1.351, P.1, B.1.427, B.1.429, B.1.167); and (vii) inhibits SARS virus infection, optionally, wherein the SARS infection is SARS-CoV-2 infection.

### Structurally-Stabilized Peptides of ACE2 α1 Helix

Disclosed herein are structurally-stabilized (e.g., stapled or stitched) ACE2 α1 helix peptides. In some instances, the structurally-stabilized (e.g., stapled or stitched) ACE2 α1 helix peptides are derived from ACE2 α1 helix peptides (IEEQAKTFLDKFNHEAEDLFYQSSLA (SEQ ID NO:1)). In some instances, the structurally-stabilized (e.g., stapled or stitched) ACE2 α1 helix peptides are derived from ACE2 α1 helix peptides (IEEQAKTFLDKFNHEAEDLFYQSS (SEQ ID NO:21)). In some instances, the structurally-stabilized (e.g., stapled or stitched) ACE2 α1 helix peptides are derived from SEQ ID NO:1. In some instances, the structurally-stabilized (e.g., stapled or stitched) ACE2 α1 helix peptides are derived from SEQ ID NO:76. In some instances, the structurally-stabilized (e.g., stapled or stitched) ACE2 α1 helix peptides are derived from SEQ ID NO:77. In some instances, the structurally-stabilized (e.g., stapled or stitched) ACE2 α1 helix peptides are derived from any one of SEQ ID NOs.: 112, 113, 117, 118, 123, 125, or 127. In some instances, the structurally-stabilized (e.g., stapled or stitched) ACE2 α1 helix peptides are variants of any one of SEQ ID NOs.: 1, 21, 76, 77, 112, 113, 117, 118, 123, 125, or 127. Such variants differ from the recited sequence at 1, 2, 3, 4, 5, 6, 7, 8, 9, 10, 11, 12, 13, 14, 15, or 16 amino acids and have one or more (e.g., 1, 2, 3, 4, 5, 6, 7) of the following properties: (i) binds the peptide of SEQ ID NO: 64 or SEQ ID NO: 65; (ii) inhibits interaction between human ACE2 protein and the S1 protein subunit of SARS-CoV-2; (iii) inhibits interaction between the carboxypeptidase domain of human ACE2 protein and the S1 protein subunit of SARS-CoV-2; (iv) inhibits interaction between the carboxypeptidase domain of human ACE2 protein and the RBD of the S1 protein subunit of SARS-CoV-2; (v) competes for human ACE2-SARS-CoV-2 S1 protein subunit binding; (vi) binds the S1 protein subunit (e.g., the RBD) of a SARS-CoV-2 variant (e.g., one or more of B.1.1.7, B.1.351, P.1, B.1.427, B.1.429, B.1.167); and (vii) inhibits SARS virus infection, optionally, wherein the SARS infection is SARS-CoV-2 infection.

In some instances, the structurally-stabilized (e.g., stapled or stitched) ACE2 α1 helix peptides has an interacting face (i.e., the face of the helix that interacts with the RBD of the virus) comprising positions 3 (corresponding to E23 of SEQ ID NO:22), 4 (Q24), 7 (T27), 10 (D30), 11 (K31), 14 (H34), 15 (E35), 21 (Y41), 22 (Q42), and 25 (L45) of SEQ ID NO:1. In some cases, the amino acids of the interacting face are not substituted. In some cases 1 to 10, 1 to 9, 1 to 8, 1 to 7, 1 to 6, 1 to 5, 1 to 4, 1 to 3, 1 to 2, or 1 amino acids of the interacting face are substituted (e.g., conservative amino acid substitutions or to alanine). In some instances, the structurally-stabilized (e.g., stapled or stitched) ACE2 α1 helix peptides has a non-interacting face (i.e., the face of the helix that does not interact with the RBD of the virus) comprising positions 1 (corresponding to 121 of SEQ ID NO:22), 2 (E22), 5 (A25), 6 (K26), 8 (F28), 9 (L29), 12 (F32), 13 (N33), 16 (A36), 17 (E37), 18 (D38), 19 (L39), 20 (F40), 23 (S43), 24 (S44), and 26 (A46) of SEQ ID NO:1. In some cases 1 to 16, 1 to 15, 1 to 14, 1 to 13, 1 to 12, 1 to 11, 1 to 12, 1 to 11, 1 to 10, 1 to 9, 1 to 8, 1 to 7, 1 to 6, 1 to 5, 1 to 4, 1 to 3, 1 to 2, or 1 amino acids of the non-interacting face are substituted (e.g., non-conservative or conservative amino acid substitutions). In some cases, the amino acids of the non-interacting face at one or more of positions 2, 5, 8, 9, 12, 13, and 14 are substituted. In some cases, 1 to 7, 1 to 6, 1 to 5, 1 to 4, 1 to 3, 1 to 2, or 1 of the amino acids of the non-interacting face are substituted. In some cases, the substitutions are non-conservative amino acid substitutions. In other cases, the substitutions are conservative amino acid substitutions. In some cases, the substitutions include both conservative and non-conservative amino acid substitutions.

In one aspect, this disclosure features an internally cross-linked ACE2h1 peptide that binds to both the S1 protein and/or RBD of SARS-CoV-2 and the S1 protein and/or RBD of one or more SARS-CoV-2 variants, optionally wherein the SARS-CoV-2 variant B.1.1.7, B.1.351, P.1, B.1.427, B.1.429, or B.1.167. In some instances, the internally cross-linked peptide has an amino acid sequence that differs from any one of SEQ ID NOs.: 77, 90-95, 98-100, 105-108, 110, 112, 113, 117, 118, 123, 125, or 127 at 2, 3, 4, 5, 6, 7, or 8 amino acid positions. In some instances, the internally cross-linked peptide comprises α, α-disubstituted non-natural amino acids with olefinic side chains that are internally cross-linked, wherein the α, α-disubstituted non-natural amino acids are inserted at one or more of (i)-(vi): (i) positions 5 and 12, (ii) positions 11 and 18, (iii) positions 12 and 19, (iv) positions 14 and 18, (v) positions 15 and 19, or (vi) positions 16 and 20, wherein the position numbering is provided based on the N-terminal E (position 1) to the C-terminal Q (position 20) of SEQ ID NO:77. In some cases, the peptides is 20 to 25, 20 to 30, 20 to 35, 20 to 40, 20 to 45, 20 to 50, 20 to 60, to 70, 20 to 80, 20 to 90, or 20 to 100 amino acids in length. Also featured are pharmaceutical compositions comprising the above internally cross-linked peptide and a pharmaceutically acceptable carrier. These internally cross-linked peptides can be used to treat or prevent a coronavirus infection (e.g., SARS-CoV-1, SARS-CoV-2, HcoV-NL63). These internally cross-linked peptides can be used to treat or prevent post-acute sequelae of SARS-CoV-2. These internally cross-linked peptides are especially useful to treat or prevent infection by variants of SARS-CoV-2.

In some instances, the structurally stabilized (e.g., stapled or stitched) ACE2 α1 helix peptides derived from SEQ ID NO:1 or 21 include SAH-ACE2h1-1-SAH-ACE2h-land SAH-ACE2h1-21-SAH-ACE2h1-24 (e.g., SEQ ID NOs: 2-12, 17-20, 51, 52, 55, 57-60, 134-143, and 172), as shown in Table 3 below. In some instances, the structurally stabilized (e.g., stapled or stitched) ACE2 α1 helix peptides derived from SEQ ID NO:76 include SEQ ID NOs: 78-111, as shown in Table 3 below. In some instances, the structurally stabilized (e.g., stapled or stitched) ACE2 α1 helix peptides derived from SEQ ID NO:21 include SEQ TD NOs: 112-126, as shown in Table 3 below. In some instances, the structurally stabilized (e.g., stapled or stitched) ACE2 α1 helix peptides derived from SEQ TD NO:77 include SEQ ID NOs: 127-133, as shown in Table 3 below.

TABLE 3
ACE2 Helix 1 Peptide Analogs.

| Name | Sequence | SEQ ID NO: |
|---|---|---|
| SAH- | X₁EEQX₂KTFLDKFNHEAEDLFYQSSXaa₁Xaa₂, | 2 |
| ACE2h1- | wherein X₁ and X₂ are non-natural amino acids with | |
| 1 | olefinic side chains; Xaa₁ = L or absent; Xaa₂ = A or | |
| | absent | |
| SAH- | IEEQX₁KTFX₂DKFNHEAEDLFYQSSXaa₁Xaa₂, | 3 |
| ACE2h1- | wherein X₁ and X₂ are non-natural amino acids with | |
| 2 | olefinic side chains; Xaaₗ = L or absent; Xaa₂ = A or | |
| | absent | |
| SAH- | IEEQAKTX₁LDKX₂NHEAEDLFYQSSXaa₁Xaa₂, | 4 |
| ACE2h1- | wherein X₁ and X₂ are non-natural amino acids with | |
| 3 | olefinic side chains; Xaa₁ = L or absent; Xaa₂ = A or | |
| | absent | |
| SAH- | IEEQAKTFLDKX₁NHEX₂EDLFYQSSXaa₁Xaa₂, | 5 |
| ACE2h1- | wherein X₁ and X₂ are non-natural amino acids with | |
| 4 | olefinic side chains; Xaa₁ = L or absent; Xaa₂ = A or | |
| | absent | |
| SAH- | IEEQAKTFLDKFNHEX₁EDLX₂YQSSXaa₁Xaa₂, | 6 |
| ACE2h1- | wherein X₁ and X₂ are non-natural amino acids with | |
| 5 | olefinic side chains; Xaa₁ = L or absent; Xaa₂ = A or | |
| | absent | |
| SAH- | IEEQAKTF8DKFNHEXEDLFYQSSXaa₁Xaa₂, | 7 |
| ACE2h1- | wherein 8 and X are non-natural amino acids with | |
| 6 | olefinic side chains; Xaa₁ = L or absent; Xaa₂ = A or | |
| | absent | |
| SAH- | IEEQ8KTFLDKXNHEAEDLFYQSSXaa₁Xaa₂, | 8 |
| ACE2h1- | wherein 8 and X are non-natural amino acids with | |
| 7 | olefinic side chains; Xaa₁ = L or absent; Xaa₂ = A or | |
| | absent | |
| SAH- | IEEQAKTFLDK8NHEAEDXFYQSSXaa₁Xaa₂, | 9 |
| ACE2h1- | wherein 8 and X are non-natural amino acids with | |
| 8 | olefinic side chains; Xaa₁ = L or absent; Xaa₂ = A or | |
| | absent | |
| SAH- | X₁EEQX₂KTFLDKFNHEX₃EDLX₄YQSSXaa₁Xaa₂, | 10 |
| ACE2h1- | wherein X₁, X₂, X₃, and X₄ are non-natural amino | |
| 9 | acids with olefinic side chains; Xaa₁ = L or absent; | |
| | Xaa₂ = A or absent | |
| SAH- | X₁EEQX₂KTFLDKX₃NHEX₄EDLFYQSSXaa₁Xaa₂, | 11 |
| ACE2h1- | wherein X₁, X₂, X₃, and X₄ are non-natural amino | |
| 10 | acids with olefinic side chains; Xaa₁ = L or absent; | |
| | Xaa₂ = A or absent | |
| SAH- | IEEQX₁KTFX₂DKFNHEX₃EDLX4YQSSXaa₁Xaa₂, | 12 |
| ACE2h1- | wherein X₁, X₂, X₃, and X₄ are non-natural amino | |
| 11 | acids with olefinic side chains; Xaa₁ = L or absent; | |
| | Xaa₂ = A or absent | |
| SAH- | Xₐₐ₁EEQ Xₐₐ₂K Xₐₐ₃ Xₐₐ₄ Xₐₐ₅DK Xₐₐ₆ Xₐₐ₇HE Xₐₐ₈ED | 17 |
| ACE2h1- | Xₐₐ₉ Xₐₐ₁₀YXₐₐ₁₁ Xₐₐ₁₂ Xₐₐ₁₃ Xₐₐ₁₄ Xₐₐ₁₅, wherein | |
| 21 | Xₐₐ₁ = I, A, or a stapling amino acid | |
| | Xₐₐ₂ = A or a stapling amino acid | |
| | Xₐₐ₃ = T, E, or F | |
| | Xₐₐ₄ = F, A, or a stapling amino acid | |
| | Xₐₐ₅ = L, A, or a stapling amino acid | |
| | Xₐₐ₆ = F, A, or a stapling amino acid | |
| | Xₐₐ₇ = N or A | |
| | Xₐₐ₈ = A or a stapling amino acid | |
| | Xₐₐ₉ = L, A, or a stapling amino acid | |
| | Xₐₐ₁₀ = F, A, or a stapling amino acid | |
| | Xₐₐ₁₁ = Q or Y | |
| | Xₐₐ₁₂ = S or A | |
| | Xₐₐ₁₃ = S or A | |
| | Xₐₐ₁₄ = L, A, or absent | |
| | Xₐₐ₁₅ = A or absent | |
| SAH- | X₁EEQX₂KTFLDKFNHEAEDLFYQSS | 172 |
| ACE2h1- | | |
| 1-alt | | |
| SAH- | IEEQX₁KTFX₂DKFNHEAEDLFYQSS | 134 |
| ACE2h1- | | |
| 2-alt | | |
| SAH- | IEEQAKTX₁LDKX₂NHEAEDLFYQSS | 135 |
| ACE2h1- | | |
| 3-alt | | |
| SAH- | IEEQAKTFLDKX₁NHEX₂EDLFYQSS | 136 |
| ACE2h1- | | |
| 4-alt | | |
| SAH- | IEEQAKTFLDKFNHEX₁EDLX₂YQSS | 137 |
| ACE2h1- | | |
| 5-alt | | |
| SAH- | IEEQAKTF8DKFNHEXEDLFYQSS | 138 |
| ACE2h1- | | |
| 6-alt | | |
| SAH- | IEEQ8KTFLDKXNHEAEDLFYQSS | 139 |
| ACE2h1- | | |
| 7-alt | | |
| SAH- | IEEQAKTFLDK8NHEAEDXFYQSS | 140 |
| ACE2h1- | | |
| 8-alt | | |
| SAH- | X₁EEQX₂KTFLDKFNHEX₃EDLX₄YQSS | 141 |
| ACE2h1- | | |
| 9-alt | | |
| SAH- | X₁EEQX₂KTFLDKX₃NHEX₄EDLFYQSS | 142 |
| ACE2h1- | | |
| 10-alt | | |
| SAH- | IEEQX₁KTFX₂DKFNHEX₃EDLX₄YQSS | 143 |
| ACE2h1- | | |
| 11-alt | | |
| SAH- | Xₐₐ₁Xₐₐ₂EQ Xₐₐ₃ Xₐₐ₄ T Xₐₐ₅ Xₐₐ₆DK Xₐₐ₇ Xₐₐ₈HE Xₐₐ₉ | 18 |
| ACE2h1- | Xₐₐ₁₀ Xₐₐ₁₁ Xₐₐ₁₂ Xₐₐ₁₃YQ Xₐₐ₁₄ Xₐₐ₁₅ LXₐₐ₁₆, wherein | |
| 22 | Xₐₐ₁ = I, A, or a stapling amino acid | |
| | Xₐₐ₂ = E or A | |
| | Xₐₐ₃ = A or a stapling amino acid | |
| | Xₐₐ₄ = K, R, or A | |
| | Xₐₐ₅ = F, A, or a stapling amino acid | |
| | Xₐₐ₆ = L, A, or a stapling amino acid | |
| | Xₐₐ₇ = F, A, or a stapling amino acid | |
| | Xₐₐ₈ = N or A | |
| | Xₐₐ₉ = A or a stapling amino acid | |
| | Xₐₐ₁₀ = E, D, or A | |
| | Xₐₐ₁₁ = D, E, or A | |
| | Xₐₐ₁₂ = L, A, or a stapling amino acid | |
| | Xₐₐ₁₃ = F, A, or a stapling amino acid | |
| | Xₐₐ₁₄ = S or A | |
| | Xₐₐ₁₅ = S or A | |
| | Xₐₐₗ₆ = A or absent | |
| SAH- | IEEQAK₆TFLD₁₀K₁₁FNHEAED18LFYQSSLA, | 19 |
| ACE2h1- | wherein K₆ and D₁₀ are linked by a lactam bridge; or | |
| 23 | wherein K₁₁ and D₁₈ are linked by a lactam bridge | |
| SAH- | Xₐₐ₁EEQ Xₐₐ₂ K₆ Xₐₐ₃ Xₐₐ₄ Xₐₐ₅ D₁₀ K₁₁ Xₐₐ₆ Xₐₐ₇HE | 20 |
| ACE2h1- | Xₐₐ₈E D₁₈ Xₐₐ₉ Xₐₐ₁₀ YXₐ₁₁ Xₐₐ₁₂ Xₐₐ₁₃ Xₐₐ₁₄ Xₐₐ₁₅, | |
| 24 | wherein | |
| | Xₐₐ₁ = I or A, | |
| | Xₐₐ₂ = A | |
| | Xₐₐ₃ = T, E, or F | |
| | Xₐₐ₄ = F or A | |
| | Xₐₐ₅ = L or A | |
| | Xₐₐ₆ = F or A | |
| | Xₐₐ₇ = N or A | |
| | Xₐₐ₈ = A = | |
| | Xₐₐ₉ = L or A | |
| | Xₐₐ₁₀ = F or A | |
| | Xₐₐ₁₁ = Q or Y | |
| | Xₐₐ₁₂ = S or A | |
| | Xₐₐ₁₃ = S or A | |
| | Xₐₐ₁₄ = L or A | |
| | Xₐₐ₁₅ = A or absent, | |
| | wherein K₆ and D₁₀ are linked by a lactam bridge; or | |
| | wherein K₁₁ and D₁₈ are linked by a lactam bridge | |
| SAH- | XEEQXKTFLDKFNHEAEDLFYQS | 78 |
| ACE2h1- | | |
| (21-43)- | | |
| A | | |
| SAH- | IXEQAXTFLDKFNHEAEDLFYQS | 79 |
| ACE2h1- | | |
| (21-43)- | | |
| B | | |
| SAH- | IEXQAKXFLDKFNHEAEDLFYQS | 80 |
| ACE2h1- | | |
| (21-43)- | | |
| C | | |
| SAH- | IEEQXKTFXDKFNHEAEDLFYQS | 81 |
| ACE2h1- | | |
| (21-43)- | | |
| D | | |
| SAH- | IEEQAXTFLXKFNHEAEDLFYQS | 82 |
| ACE2h1- | | |
| (21-43)- | | |
| E | | |
| SAH- | IEEQAKXFLDXFNHEAEDLFYQS | 83 |
| ACE2h1- | | |
| (21-43)- | | |
| F | | |
| SAH- | IEEQAKTXLDKXNHEAEDLFYQS | 84 |
| ACE2h1- | | |
| (21-43)- | | |
| G | | |
| SAH- | IEEQAKTFXDKFXHEAEDLFYQS | 85 |
| ACE2h1- | | |
| (21-43)- | | |
| H | | |
| SAH- | IEEQAKTFLXKFNXEAEDLFYQS | 86 |
| ACE2h1- | | |
| (21-43)- | | |
| I | | |
| SAH- | IEEQAKTFLDXFNHXAEDLFYQS | 87 |
| ACE2h1- | | |
| (21-43)- | | |
| J | | |
| SAH- | IEEQAKTFLDKXNHEXEDLFYQS | 88 |
| ACE2h1- | | |
| (21-43)- | | |
| K | | |
| SAH- | IEEQAKTFLDKFXHEAXDLFYQS | 89 |
| ACE2h1- | | |
| (21-43)- | | |
| L | | |
| SAH- | IEEQAKTFLDKFNXEAEXLFYQS | 90 |
| ACE2h1- | | |
| (21-43)- | | |
| M | | |
| SAH- | IEEQAKTFLDKFNHXAEDXFYQS | 91 |
| ACE2h1- | | |
| (21-43)- | | |
| N | | |
| SAH- | IEEQAKTFLDKFNHEXEDLXYQS | 92 |
| ACE2h1- | | |
| (21-43)- | | |
| O | | |
| SAH- | IEEQAKTFLDKFNHEAXDLFXQS | 93 |
| ACE2h1- | | |
| (21-43)- | | |
| P | | |
| SAH- | IEEQAKTFLDKFNHEAEXLFYXS | 94 |
| ACE2h1- | | |
| (21-43)- | | |
| Q | | |
| SAH- | IEEQAKTFLDKFNHEAEDXFYQX | 95 |
| ACE2h1- | | |
| (21-43)- | | |
| R | | |
| SAH- | 8EEQAKTXLDKFNHEAEDLFYQS | 96 |
| ACE2h1- | | |
| (21-43)- | | |
| S | | |
| SAH- | I8EQAKTFXDKFNHEAEDLFYQS | 97 |
| ACE2h1- | | |
| (21-43)- | | |
| T | | |
| SAH- | IE8QAKTFLXKFNHEAEDLFYQS | 98 |
| ACE2h1- | | |
| (21-43)- | | |
| U | | |
| SAH- | IEE8AKTFLDXFNHEAEDLFYQS | 99 |
| ACE2h1- | | |
| (21-43)- | | |
| V | | |
| SAH- | IEEQ8KTFLDKXNHEAEDLFYQS | 100 |
| ACE2h1- | | |
| (21-43)- | | |
| W | | |
| SAH- | IEEQA8TFLDKFXHEAEDLFYQS | 101 |
| ACE2h1- | | |
| (21-43)- | | |
| X | | |
| SAH- | IEEQAK8FLDKFNXEAEDLFYQS | 102 |
| ACE2h1- | | |
| (21-43)- | | |
| Y | | |
| SAH- | IEEQAKT8LDKFNHXAEDLFYQS | 103 |
| ACE2h1- | | |
| (21-43)- | | |
| Z | | |
| SAH- | IEEQAKTF8DKFNHEXEDLFYQS | 104 |
| ACE2h1- | | |
| (21-43)-a | | |
| SAH- | IEEQAKTFL8KFNHEAXDLFYQS | 105 |
| ACE2h1- | | |
| (21-43)-b | | |
| SAH- | IEEQAKTFLD8FNHEAEXLFYQS | 106 |
| ACE2h1- | | |
| (21-43)-c | | |
| SAH- | IEEQAKTFLDK8NHEAEDXFYQS | 107 |
| ACE2h1- | | |
| (21-43)-d | | |
| SAH- | IEEQAKTFLDKF8HEAEDLXYQS | 108 |
| ACE2h1- | | |
| (21-43)-e | | |
| SAH- | IEEQAKTFLDKFN8EAEDLFXQS | 109 |
| ACE2h1- | | |
| (21-43)-f | | |
| SAH- | IEEQAKTFLDKFNH8AEDLFYXS | 110 |
| ACE2h1- | | |
| (21-43)-g | | |
| SAH- | IEEQAKTFLDKFNHE8EDLFYQX | 111 |
| ACE2h1- | | |
| (21-43)-h | | |
| SAH- | IEEQXKTAXDKANHEAEDAAYQSAXaa₁Xaa₂, | 51 |
| ACE2h1- | wherein X is a non-natural amino acid with olefinic | |
| mut-2 | side chains, Xaa₁ is L, A, or absent, and Xaa₂ is A or | |
| | absent | |
| SAH- | IEEQXKTAXDKANHEXEDAXYQSAXaa₁Xaa₂, | 52 |
| ACE2h1- | wherein X is a non-natural amino acid with olefinic | |
| mut-3 | side chains, wherein Xaa₁ is L, A, or absent, and Xaa₂ | |
| | is A or absent | |
| SAH- | QEEQXKDAXDHANHEXEYQXYQSAXaa₁Xaa₂, | 55 |
| ACE2h1- | wherein X is a non-natural amino acid with olefinic | |
| mut-6 | side chains, Xaa₁ is L, A, or absent, and Xaa₂ is A or | |
| | absent | |
| SAH- | IEEQAKTXADKXNHEAEQAAYQSAXaa₁Xaa₂, | 57 |
| ACE2h1- | wherein X is a non-natural amino acid with olefinic | |
| mut-8 | side chains, Xaa₁ is L, A, or absent, and Xaa₂ is A or | |
| | absent | |
| SAH- | IEEQXKTAXDKANHEXEQAXYQSAXaa₁Xaa₂, | 58 |
| ACE2h1- | wherein X is a non-natural amino acid with olefinic | |
| mut-9 | side chains, Xaa₁ is L, A, or absent, and Xaa₂ is A or | |
| | absent | |
| SAH- | IEEQXKEAXDKANHEXEQAXYQSAXaa₁Xaa₂, | 59 |
| ACE2h1- | wherein X is a non-natural amino acid with olefinic | |
| mut-10 | side chains, Xaa₁ is L, A, or absent, and Xaaz is A or | |
| | absent | |
| SAH- | IEEQAKTA8DKANHEXEQAAYQSAXaa₁Xaa₂, | 60 |
| ACE2h1- | wherein 8 and X are non-natural amino acids with | |
| mut-11 | olefinic side chains; Xaa₁ = L, A, or absent; Xaa₂ = | |
| | A or absent | |
| SAH- | IEEQAKTFLDKFNHEAEDLFYQSA | 112 |
| ACE2h1- | | |
| (21-44)- | | |
| mut-12 | | |
| SAH- | IEEQAKTAADKANHEAEDAAYQSA | 113 |
| ACE2h1- | | |
| (21-44)- | | |
| mut-13 | | |
| SAH- | IEEQXKTAXDKANHEAEDAAYQSA | 114 |
| ACE2h1- | | |
| (21-44)- | | |
| mut- | | |
| 13(D) | | |
| SAH- | IEEQXKTAXDKANHEXEDAXYQSA | 115 |
| ACE2h1- | | |
| (21-44)- | | |
| mut- | | |
| 13(D, O) | | |
| SAH- | IEEQXKEAXDKANHEXEDAXYQSA | 116 |
| ACE2h1- | | |
| (21-44)- | | |
| mut- | | |
| 14(D, O) | | |
| SAH- | AEEQAKTAADKAAHEAEQAAYQAA | 117 |
| ACE2h1- | | |
| (21-44)- | | |
| mut-15 | | |
| SAH- | IEEQAKTAADKANHEAEQAAYQSA | 118 |
| ACE2h1- | | |
| (21-44)- | | |
| mut-16 | | |
| SAH- | IEEQAKTXADKXNHEAEQAAYQSA | 119 |
| ACE2h1- | | |
| (21-44)- | | |
| mut- | | |
| 16(G) | | |
| SAH- | IEEQXKTAXDKANHEXEQAXYQSA | 120 |
| ACE2h1- | | |
| (21-44)- | | |
| mut- | | |
| 16(D, O) | | |
| SAH- | IEEQXKEAXDKANHEXEQAXYQSA | 121 |
| ACE2h1- | | |
| (21-44)- | | |
| mut- | | |
| 17(D, O) | | |
| SAH- | IEEQAKTA8DKANHEXEQAAYQSA | 122 |
| ACE2h1- | | |
| (21-44)- | | |
| mut- | | |
| 18(D, O) | | |
| SAH- | IQEQAKTDADKHNHEAEDYQYQSA | 123 |
| ACE2h1- | | |
| (21-44)- | | |
| mut-19 | | |
| SAH- | IQEQXKTDXDKHNHEAEDYQYQSA | 124 |
| ACE2h1- | | |
| (21-44)- | | |
| mut-20 | | |
| SAH- | QEEQAKDAADHANHEAEYQAYQSA | 125 |
| ACE2h1- | | |
| (21-43)- | | |
| mut-21 | | |
| SAH- | QEEQXKDAXDHANHEXEYQXYQSA | 126 |
| ACE2h1- | | |
| (21-43)- | | |
| mut- | | |
| 21(D, O) | | |
| SAH- | ETVDFFAEWFDVEAEDKDYL | 127 |
| ACE2h1- | | |
| (23-42)- | | |
| mut-22 | | |
| SAH- | ETXDFFXEWADVXAEDXDYL | 128 |
| ACE2h1- | | |
| (23-42)- | | |
| mut- | | |
| 23(D, N) | | |
| SAH- | ETXDFFXEWADVEXEDKXYL | 129 |
| ACE2h1- | | |
| (23-42)- | | |
| mut- | | |
| 23(D, O) | | |
| SAH- | ETXDFFXEWFDVXAEDXDYL | 130 |
| ACE2h1- | | |
| (23-42)- | | |
| mut- | | |
| 22(D, N) | | |
| SAH- | ETXDFEXEWFDVXAEDXDYL | 131 |
| ACE2h1- | | |
| (23-42)- | | |
| mut- | | |
| 24(D, N) | | |
| SAH- | ETXDFLXEWFDVXAEDXDYL | 132 |
| ACE2h1- | | |
| (23-42)- | | |
| mut- | | |
| 25(D, N) | | |
| SAH- | ETXDFYXEWFDVXAEDXDYL | 133 |
| ACE2h1- | | |
| (23-42)- | | |
| mut- | | |
| 26(D, N) | | |

In Table 3, X₁, Xa, X₃, X₄, 8, and X are all α,α-disubstituted non-natural amino acids with olefinic side chains (which can be cross-linked by e.g., a RCM reaction). R₈ and S5 are α-methylated and the final stabilized (e.g., stapled) peptide is produced by ring-closing metathesis (and loss of ethylene). In some instances, X₁, X₂, X₃, X₄=(4′-pentenyl)alanine or (S)-pentenyl alanine (S5); 8=2-(7′-octenyl)alanine or (R)-octenyl alanine residues (R8); and X=(4′-pentenyl)alanine or S5.

In some instances (e.g., SEQ ID NOs: 2-9, 51, 57, and 60), the structurally-stabilized peptide is single-stapled peptide. In some instances (e.g., SEQ ID NOs: 10-12, 52, 55, 58, and 59) the structurally-stabilized peptide is a double-stapled peptide. In some instances (e.g., SEQ TD NO: 17 or 18), the structurally-stabilized peptide is stapled peptide and includes 1 or 2 staples. In some instances (e.g., SEQ TD NO: 19 or 20), the structurally-stabilized peptide includes lactam bridge links between a lysine (K) and an aspartic acid (D) of the ACE2 α1 helix or a variant thereof.

In some instances, each peptide in Table 3 can include beta alanine at the N-terminus. In some embodiments, a conjugate can be coupled to the N-terminus of the peptides in Table 3. In some instances, the conjugate is a detection moiety disclosed herein (e.g., FITC (fluorescein isothiocyanate)). In some instances, the conjugate is a capture moiety disclosed herein (e.g., a biotin moiety).

In some instances, the stapled peptide is an internally cross-linked peptide of any one of the sequences set forth in any one of SEQ ID NOs.:78-111, 114-116, 119-122, 124, 126, or 128-133.

In some instances, disclosed herein is a stapled or stitched peptide comprising or consisting of any one of the amino acids sequences of SEQ ID NO: 1, 21, 76, 77, 112, 113, 117, 118, 123, 125, or 127, except that at least two (e.g., 2, 3, 4, 5, 6) amino acids of SEQ ID NO: 1 or 21 are replaced with a non-natural amino acid capable of forming a staple or stitch. In some instances, the non-natural amino acid is an α, α-disubstituted non-natural amino acids with olefinic side chains. In some instances, the stapled peptide of SEQ ID NO: 1, 21, 76, 77, 112, 113, 117, 118, 123, 125, or 127 further includes 1, 2, 3, 4, 5, 6, 7, 8, 9, 10, 11, or 12 amino acid substitutions and can bind the S1 protein and/or RBD of SARS-CoV-2 or a SARS-CoV-2 variant.

In some instances, the non-natural amino acids that may be used as stapling amino acids or stitching amino acids are: (R)-2-(7′-octenyl)alanine; (R)-2-(4′-pentenyl)alanine; (R)-α-(7′-octenyl)alanine; (R)-α-(4′-pentenyl)alanine; (S)-α-(7′-octenyl)alanine; (S)-2-(7′-octenyl)alanine; (S)-α-(4′-pentenyl)alanine; (S)-2-(4′-pentenyl)alanine; α,α-Bis(4′-pentenyl)glycine; and α,α-Bis(7′-octeny)glycine.

In some embodiments, an internal staple replaces the side chains of 2 amino acids, i.e., each staple is between two amino acids separated by, for example, 2, 3, or 6 amino acids. In some embodiments, an internal stitch replaces the side chains of 3 amino acids, i.e., the stitch is a pair of crosslinks between three amino acids separated by, for example, 2, 3, or 6 amino acids. In some embodiments, the amino acids forming the staple or stitch are at each of positions i and i+3 of the staple. In some embodiments, the amino acids forming the staple or stitch are at each of positions i and i+4 of the staple. In some embodiments, the amino acids forming the staple or stitch are at each of positions i and i+7 of the staple. For example, where a peptide has the sequence . . . X₁, X₂, X₃, X₄, X₅, X₆, X₇, X₈, X₉ . . . , cross-links between X₁ and X₄ (i and i+3), or between X₁ and X₅ (i and i+4), or between X₁ and X₈ (i and i+7) are useful hydrocarbon stapled forms of that peptide. The use of multiple cross-links (e.g., 2, 3, 4, or more) is also contemplated. Additional description regarding making and use of hydrocarbon-stapled peptides can be found, e.g., in U.S. Patent Publication Nos. 2012/0172285, 2010/0286057, and 2005/0250680, the contents of which are incorporated by reference herein in their entireties.

In some embodiments, the amino acids forming the staple or stitch are at each of positions i and i+4 of the staple. In some instances, to achieve the various staple lengths, α-methyl, α-alkenyl amino acids may be installed at i, i+4 positions using two (S)-pentenyl alanine residues (S5). In some instances, α-methyl, α-alkenyl amino acids may be installed at i, i+4 positions using (R)-octenyl alanine residues (R₈). In some embodiments, the amino acids forming the staple or stitch are at each of positions i and i+7 of the staple. In some instances, to achieve the various staple lengths, α-methyl, α-alkenyl amino acids may be installed at i, i+7 positions using one (S)-pentenyl alanine residues (S5) at a first position i and one (R)-octenyl alanine residues (R8) at a second position i+7. In one instance, to achieve the various staple lengths, α-methyl, α-alkenyl amino acids may be installed at i, i+7 positions using one (R)-octenyl alanine residues (R8) at a first position i and one (S)-pentenyl alanine residues (S5) at a second position i+7.

“Peptide stapling” is a term coined from a synthetic methodology wherein two olefin-containing side-chains (e.g., cross-linkable side chains) present in a peptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). The structural-stabilization may be by, e.g., stapling the peptide (see, e.g., Walensky, J. Med. Chem., 57:6275-6288 (2014), the contents of which are incorporated by reference herein in its entirety). In some cases, the staple is a hydrocarbon staple.

In some instances, the structural-stabilization is a stitch. The term “peptide stitching,” as used herein, refers to multiple and tandem stapling events in a single peptide chain to provide a “stitched” (e.g., tandem or multiply stapled) peptide, in which two staples, for example, are linked to a common residue. Peptide stitching is disclosed, e.g., in WO 2008/121767 and WO 2010/068684, which are both hereby incorporated by reference in their entirety.

In some instances, a staple or stitch used herein is a lactam staple or stitch. In some instances, the lactam staple or stitch couples a lysine residue side chain and to an aspartic acid or glutamic acid residues side-chain.

In some instances, a staple or stitch used herein is a UV-cycloaddition staple or stitch; an oxime staple or stitch; a thioether staple or stitch; a double-click staple or stitch; a bis-lactam staple or stitch; a bis-arylation staple or stitch; or a combination of any two or more thereof. Stabilized peptides as described herein include stapled peptides and stitched peptides as well as peptides containing multiple stitches, multiple staples or a mix of staples and stitches, or any other chemical strategies for structural reinforcement (see, e.g., Balaram P. Cur. Opin. Struct. Biol. 1992; 2:845; Kemp D S, et al., J. Am. Chem. Soc. 1996; 118:4240; Orner B P, et al., J. Am. Chem. Soc. 2001; 123: 5382; Chin J W, et al., Int. Ed. 2001; 40:3806; Chapman R N, et al., J. Am. Chem. Soc. 2004; 126: 12252; Horne W S, et al., Chem., Int. Ed. 2008; 47:2853; Madden et al., Chem Commun (Camb). 2009 Oct. 7; (37): 5588-5590; Lau et al., Chem. Soc. Rev., 2015, 44:91-102; and Gunnoo et al., Org. Biomol. Chem., 2016, 14:8002-8013; each of which is incorporated by reference herein in its entirety).

A peptide is “structurally-stabilized” in that it maintains its native secondary structure. For example, stapling allows a peptide, predisposed to have an α-helical secondary structure, to maintain its native α-helical conformation. This secondary structure increases resistance of the peptide to proteolytic cleavage and heat, and may increase target binding affinity, hydrophobicity, and cell permeability. Accordingly, the stapled (cross-linked) peptides described herein have improved biological activity relative to a corresponding non-stapled (un-cross-linked) peptide.

In certain instances, the modification(s) to introduce structural stabilization (e.g., internal cross-linking, e.g., stapling, stitching) into the ACE2 α1 helix peptides described herein may be positioned on the face of the ACE2 α1 helix that does not interact with SARS-CoV-2 S1. In certain instances, the modification(s) to introduce structural stabilization (e.g., internal cross-linking, e.g., stapling, stitching) into the ACE2 α1 helix peptides described herein may be positioned on the face of the ACE2 α1 helix that interacts with SARS-CoV-2 S1.

In some instances, the modifications to introduce structural stabilization (e.g., internal cross-linking, e.g., stapling or stitching) into the ACE2 α1 helix peptides described herein are positioned at the amino acid positions in the ACE2 α1 helix peptide corresponding to residues:
(i) 1 and 5 of SEQ ID NO: 1 or 21;
(ii) 5 and 9 of SEQ ID NO: 1 or 21;
(iii) 8 and 12 of SEQ ID NO: 1 or 21;
(iv) 12 and 16 of SEQ ID NO: 1 or 21;
(v) 9 and 16 of SEQ ID NO: 1 or 21;
(vi) 5 and 12 of SEQ ID NO: 1 or 21;
(vii) 12 and 19 of SEQ ID NO: 1 or 21;
(viii) 1, 5, 16, and 20 of SEQ ID NO: 1 or 21;
(ix) 1, 5, 12, and 16 of SEQ ID NO: 1 or 21; or
(x) 5, 9, 16, and 20 of SEQ ID NO: 1 or 21.

In some instances, the modifications to introduce structural stabilization (e.g., internal cross-linking, e.g., stapling or stitching) into the ACE2 α1 helix peptides described herein are positioned at the amino acid positions in the ACE2 α1 helix peptide corresponding to residues 1, 5, 8, 9, 12, 16, 19, 20, or a combination thereof, of SEQ ID NO: 1 or 21.

In some instances, the modifications to introduce structural stabilization (e.g., internal cross-linking, e.g., stapling or stitching) into the ACE2 α1 helix peptides described herein are positioned at the amino acid positions in the ACE2 α1 helix peptide corresponding to residues 1, 5, 8, 9, 12, 16, 19, 20, or a combination thereof, of SEQ ID NO: 1 or 21.

In certain instances, the ACE2 α1 helix peptides described herein (e.g., SEQ ID NOs: 11-52) may also contain one or more (e.g., 1, 2, 3, 4, or 5) amino acid substitutions (relative to an amino acid sequence set forth in any one of SEQ ID NOs: 2-12 17-20, 134-143, and 172), e.g., one or more (e.g., 1, 2, 3, 4, or 5) conservative and/or non-conservative amino acid substitutions. In some instances, the ACE2 α1 helix peptides described herein (e.g., SEQ ID NOs: 2-12 17-20, 134-143, and 172) may also contain at least one, at least 2, at least 3, at least 4, or at least 5 amino acids added to the N-terminus of the peptide. In some instances, the ACE2 α1 helix peptides described herein (e.g., SEQ ID NOs: 2-12 17-20, 134-143, and 172) may also contain at least one, at least 2, at least 3, at least 4, or at least 5 amino acids added to the C-terminus of the peptide.

In one aspect, the structurally-stabilized ACE2 α1 helix peptide comprises Formula (I),

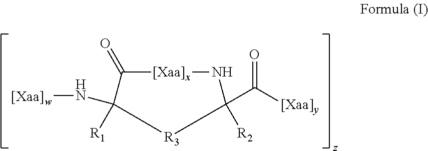

or a pharmaceutically acceptable salt thereof. In some instances, each R₁ and R₂ is independently H, alkyl, alkenyl, alkynyl, arylalkyl, cycloalkylalkyl, heteroarylalkyl, or heterocyclylalkyl, any of which is substituted or unsubstituted. In some instances, each R3 is independently alkylene, alkenylene, or alkynylene, any of which is substituted or unsubstituted;
each x is independently 2, 3, or 6. In some instances, each w and y is independently 1, 2, 3, 4, 5, 6, 7, 8, 9, 10, 11, 12, 13, 14, 15, 16, 17, 18, 19, 20, 21, 22, 23, or 24. In some instances, z is 1, 2, or 3. In some instances, each Xaa is independently an amino acid. In some instances, the stabilized peptide prevents or inhibits the interaction between Angiotensin converting enzyme 2 (ACE2) and a virus whose receptor binding domain binds ACE2. In some instances, the sequence of the peptide of pharmaceutically acceptable salt thereof has SEQ ID NO: 1 with:
   (i) at least 2 amino acids that are substituted with α, α-disubstituted non-natural amino acids with olefinic side chains that form an internal cross-link,
   (ii) 0, 1, 2, 3, 4, 5, 6, 7, 8, 9, 10, or 11 additional amino acid substitutions; and
   (iii) 0, 1, 2, 3, 4, or 5 deletions at the N and/or C-terminus of the sequence.

In some embodiments, each of the [Xaa]_{w} of Formula (I), the [Xaa]ₓ of Formula (I), and the [Xaa]_{y} of Formula (I) is as described for any one of constructs 1-8 or 12-14 in Table 4. For example, for a stabilized peptide comprising the [Xaa]_{w}, the [Xaa]ₓ, and the [Xaa]_{y} of construct 2 of Table 4, the [Xaa]_{w}, the [Xaa]ₓ, and the [Xaa]_{y} is IEEQ (SEQ ID NO: 24), KTF, and DKFNHiEAEDLFYQSSXaa1Xaa2 (SEQ ID NO: 153), respectively. As another example, for a stabilized peptide comprising the [Xaa]_{w}, the [Xaa]ₓ, and the [Xaa]_{y} of construct 3 of Table 4, the [Xaa]_{w}, the [Xaa]ₓ, and the [Xaa]_{y} is IEEQAKT (SEQ ID NO: 26), LDK, and NHEAEDLFYQSSXaa1Xaa2 (SEQ ID NO: 156), respectively.

TABLE 4
[Xaa]_{w}, [Xaa]ₓ, and [Xaa]_{y} sequences for Formula (I) constructs 1-8
and 12-14.

| Construct | [Xaa]_{w} | [Xaa]ₓ | [Xaa]_{y} |
|---|---|---|---|
| 1 (SAH- | | EEQ | KTFLDKFNHEAE(D/Q) |
| ACE2h1-1) | | | LFYQSSXaa₁Xaa₂ |
| | | | (SEQ ID NO: 23), |
| | | | wherein |
| | | | Xaa₁ = L or absent |
| | | | Xaa₂ = A or absent |
| 2 (SAH- | IEEQ (SEQ ID NO: 24) | KTF | DKFNHEAE(D/Q)LFY |
| ACE2h1-2) | | | QSS Xaa₁Xaa₂ (SEQ ID |
| | | | NO: 153), wherein |
| | | | Xaa₁ = L or absent |
| | | | Xaa₂ = A or absent |
| 3 (SAH- | IEEQAKT (SEQ ID NO: | LDK | NHEAE(D/Q)LFYQSS |
| ACE2h1-3) | 26) | | Xaa₁Xaa₂ (SEQ ID NO: |
| | | | 156), wherein |
| | | | Xaa₁ = L or absent |
| | | | Xaa₂ = A or absent |
| 4 (SAH- | IEEQAKTFLDK (SEQ | NHE | E(D/Q)LFYQSSXaa₁Xaa₂ |
| ACE2h1-4) | ID NO: 29) | | (SEQ ID NO: 31), |
| | | | wherein |
| | | | Xaa₁ = L or absent |
| | | | Xaa₂ = A or absent |
| 5 (SAH- | IEEQAKTFLDKFNHE | EDL | YQSS Xaa₁Xaa₂ (SEQ |
| ACE2h1-5) | (SEQ ID NO: 32) | | ID NO: 33), wherein |
| | | | Xaa₁ = L or absent |
| | | | Xaa₂ = A or absent |
| 6 (SAH- | IEEQAKTF (SEQ ID | DKFNHE (SEQ ID | E(D/Q)LFYQSS |
| ACE2h1-6) | NO: 34) | NO: 35) | Xaa₁Xaa₂ (SEQ ID NO: |
| | | | 36), wherein |
| | | | Xaa₁ = L or absent |
| | | | Xaa₂ = A or absent |
| 7 (SAH- | IEEQ (SEQ ID NO: 24) | KTFLDK (SEQ ID | NHEAE(D/Q)LFYQSS |
| ACE2h1-7) | | NO: 38) | Xaa₁Xaa₂ (SEQ ID |
| | | | NO: 39), wherein |
| | | | Xaa₁ = L or absent |
| | | | Xaa₂ = A or absent |
| 8 (SAH- | IEEQAKTFLDK (SEQ | NHEAE(D/Q) | FYQSSXaa₁Xaa₂ (SEQ |
| ACE2h1-8) | ID NO: 29) | (SEQ ID NO: 41) | ID NO: 42), wherein |
| | | | Xaa₁ = L or absent |
| | | | Xaa₂ = A or absent |
| 12 | IEEQ (SEQ ID NO: 24) | KTA | DKANHEAE(D/Q)AAYQ |
| | | | SAXaa₁Xaa₂ (SEQ ID |
| | | | NO: 67), wherein Xaa₁ is |
| | | | L, A, or absent, and Xaa₂ |
| | | | is A or absent |
| 13 | IEEQAKT (SEQ ID NO: | ADK | NHEAEQAAYQSAXaa₁ |
| | 26) | | Xaa₂ (SEQ ID NO: 68), |
| | | | wherein Xaa₁ is L, A, or |
| | | | absent, and Xaa₂ is A or |
| | | | absent |
| 14 | IEEQAKTA (SEQ ID | DKANHE (SEQ ID | EQAAYQSAXaa₁Xaa₂ |
| | NO: 61) | NO: 62) | (SEQ ID NO: 69), wherein |
| | | | Xaa₁ is L, A, or absent, |
| | | | and Xaa2 is A or absent |

In certain instances, the sequences set forth above in Table 4 can have at least one (e.g., 1, 2, 3, 4, 5, or 6) amino acid substitution or deletion. All of these peptides and their variants bind the RBD of the virus (e.g., SARS-CoV-2) and inhibit its interaction with ACE2 on the cell (e.g., human respiratory cell) surface. The ACE2 α1 helix peptides can include any amino acid sequence described herein.

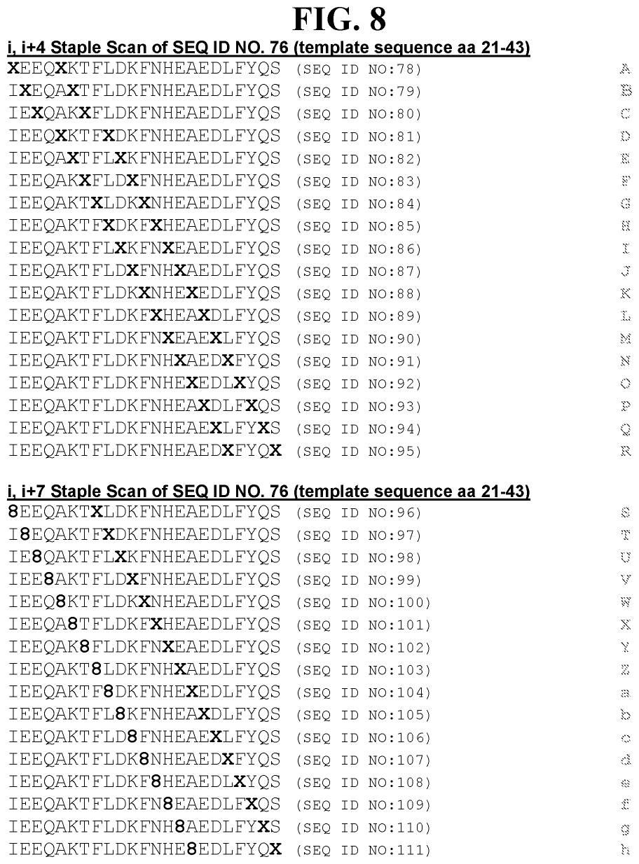

In addition to the sequences provided in Table 4, peptides comprising Formula (I) also include SEQ ID NOs: 78-111, 114, 119, and 122, as shown in FIG. 8.

In some instances, a peptide comprising Formula (I) comprising the sequences set forth above in Table 4 can have one or more of the properties listed below: (i) the peptide prevents or inhibits the interaction between Angiotensin converting enzyme 2 (ACE2) and a virus whose receptor binding domain binds ACE2; (ii) the peptide is alpha helical; (iii) the peptide is protease resistant; and/or (iv) the peptide blocks or inhibits infection of human ACE2 expressing epithelial cells. In some instances, the epithelial cells are located in the respiratory system.

In some instances, the peptide of Formula (I) has a sequence of any one of SEQ ID NOs.: 78-111, wherein [Xaa]_{w} refers to the amino acids corresponding to those before the first (N-terminal most) stapling amino acid; [Xaa]ₓ refers to the amino acids corresponding to those between the first (N-terminal most) and second (N-terminal most) stapling amino acid; and Xaa]_{y} refers to the amino acids corresponding to those after the last (C-terminal most) stapling amino acid in these sequences.

The tether of Formula (I) can include an alkyl, alkenyl, or alkynyl moiety (e.g., C₅, C₈, C₁₁, or C₁₂ alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances of Formula (I), x is 2, 3, or 6. In some instances of Formula (I), each y is independently an integer between 0 and 15, or 3 and 15. In some instances of Formula (I), R₁ and R₂ are each independently H or C₁-C₃ alkyl. In some instances of Formula (I), R₁ and R₂ are each independently C₁-C₃ alkyl. In some instances of Formula (I), at least one of R₁ and R₂ are methyl. For example, R₁ and R₂ can both be methyl. In some instances of Formula (I), R₃ is alkyl (e.g., C₈ alkyl) and x is 3. In some instances of Formula (I), R₃ is C₁₁ alkyl and x is 6. In some instances of Formula (I), R₃ is alkenyl (e.g., C₈ alkenyl) and x is 3. In some instances of Formula (I), x is 6 and R₃ is C₁₁ alkenyl. In some instances, R₃ is a straight chain alkyl, alkenyl, or alkynyl. In some instances, R₃ is CH₂—CH₂—CH₂—CH═CH—CH₂—CH₂—CH₂.

In another aspect of Formula (I), the two alpha, alpha disubstituted stereocenters are both in the R configuration or S configuration (e.g., i, i+4 cross-link), or one stereocenter is R and the other is S (e.g., i, i+7 cross-link). Thus, where Formula (I) is depicted as:

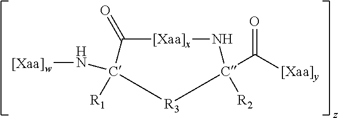

The C′ and C″ disubstituted stereocenters can both be in the R configuration or they can both be in the S configuration, e.g., when x is 3. When x is 6 in Formula (I), the C′ disubstituted stereocenter is in the R configuration and the C″ disubstituted stereocenter is in the S configuration. The R₃ double bond of Formula (I) can be in the E or Z stereochemical configuration.

In some instances of Formula (I), R₃ is [R₄—K—R₄]ₙ; and R₄ is a straight chain alkyl, alkenyl, or alkynyl.

In some instances, “z” of Formula (I) is greater than one. In some instances, z=2, as shown in Formula (II). In this instance, the peptide includes more than one staple.

In one aspect, disclosed herein is a compound comprising a stabilized peptide comprising a sequence having the formula:

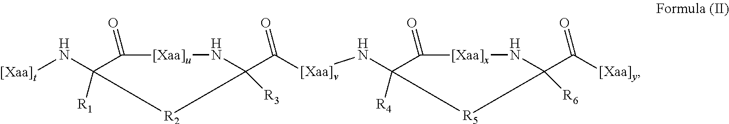

or a pharmaceutically acceptable salt thereof. In some instances, each R₁, R₃, R₄, and R₆ is independently H, alkyl, alkenyl, alkynyl, arylalkyl, cycloalkylalkyl, heteroarylalkyl, or heterocyclylalkyl, any of which is substituted or unsubstituted. In some instances, each R₂ and R₅ is independently alkylene, alkenylene, or alkynylene, any of which is substituted or unsubstituted; optionally wherein R₂ and R₅ are either C₈ alkylene, C₈ alkenylene, or C₈ alkylene; or C₁₁ alkylene, C₁₁ alkenylene, or C₁₁ alkylene. In some instances, each u and x is independently 2, 3, or 6. In some instances, each t, v, and y is independently 1, 2, 3, 4, 5, 6, 7, 8, 9, 10, 11, 12, 13, 14, 15, 16, 17, 18, 19, 20, 21, or 22. In some instances, each Xaa is independently an amino acid. In some instances, the stabilized peptide prevents or inhibits the interaction between Angiotensin converting enzyme 2 (ACE2) and a virus whose receptor binding domain binds ACE2. In some instances, the sequence of the peptide or the pharmaceutically acceptable salt thereof has SEQ ID NO: 1 with:
(i) at least 4 amino acids that are substituted with α, α-disubstituted non-natural amino acids with olefinic side chains that form an internal cross-link;
(ii) 0, 1, 2, 3, 4, 5, 6, 7, 8, 9, 10, or 11 additional amino acid substitutions, and/or
(iii) 0, 1, 2, 3, 4, or 5 amino acid deletions at the N and/or C-terminus of the sequence.

In some embodiments, each of the [Xaa]ₜ of Formula (II), the [Xaa]ᵤ of Formula (II), the [Xaa]ᵥ of Formula (II), the [Xaa]ₓ of Formula (II), and the [Xaa]_{y} of Formula (II) is as described for any one of constructs 9-11 of Table 5. For example, for a stabilized peptide comprising the [Xaa]ₜ, the [Xaa]ᵤ, the [Xaa]ᵥ, the [Xaa]ₓ, and the [Xaa]_{y} of construct 9 of Table 5, the [Xaa]ₜ, the [Xaa]ᵤ, the [Xaa]ᵥ, the [Xaa]ₓ, and the [Xaa]_{y} is absent; EEQ; KTFLDKFNHE (SEQ ID NO:43); EDL; and YQSS (SEQ ID NO: 161), respectively. As another example, for a stabilized peptide comprising the [Xaa]ₜ, the [Xaa]ᵤ, the [Xaa]ᵥ, the [Xaa]ₓ, and the [Xaa]_{y} of construct 9 of Table 5, the [Xaa]ₜ, the [Xaa]ᵤ, the [Xaa]ᵥ, the [Xaa]ₓ, and the [Xaa]_{y} is absent; EEQ; KTFLDK (SEQ ID NO:38); NHE; and EDLFYQSS (SEQ ID NO: 159), respectively.

TABLE 5
[Xaa]ₜ, [Xaa]ᵤ, [Xaa]ᵥ, [Xaa]ₓ, and [Xaa]_{y} sequences for Formula (II)
constructs 9-11 and 15-18.

| Construct | [Xaa]ₜ | [Xaa]ᵤ | [Xaa]ᵥ | [Xaa]ₓ | [Xaa]_{y} |
|---|---|---|---|---|---|
| 9 (SAH- | absent | EEQ | KTFLDKFNHE | EDL | YQSSXaa₁Xaa₂ (SEQ ID |
| ACE2h1- | | | (SEQ ID NO: | | NO: 161), wherein |
| 8) | | | 43) | | Xaa₁ = L or absent |
| | | | | | Xaa₂ = A or absent |
| 10 (SAH- | absent | EEQ | KTFLDK (SEQ | NHE | EDLFYQSS Xaa₁Xaa₂ |
| ACE2h1- | | | ID NO: 38) | | (SEQ ID NO: 159), |
| 9) | | | | | wherein |
| | | | | | Xaa₁ = L or absent |
| | | | | | Xaa₂ = A or absent |
| 11 (SAH- | IEEQ | KTF | DKFNHE (SEQ | EDL | YQSSXaa₁Xaa₂ (SEQ ID |
| ACE2h1- | (SEQ | | ID NO: 48) | | NO: 161), wherein |
| 10) | ID NO: | | | | Xaa₁ = L or absent |
| | 24) | | | | Xaa₂ = A or absent |
| 15 | IEEQ | KTA | DKANHE | EDA | YQSAXaa₁Xaa₂ (SEQ ID |
| | (SEQ | | (SEQ ID NO: | | NO: 63), wherein Xaa₁ is L, |
| | ID NO: | | 62) | | A, or absent, and Xaa₂ is A |
| | 24) | | | | or absent |
| 16 | QEEQ | KDA | DHANHE | EYQ | YQSAXaa₁Xaa₂ (SEQ ID |
| | (SEQ | | (SEQ ID NO: | | NO: 63), wherein Xaa₁ is L, |
| | ID | | 165) | | A, or absent, and Xaa₂ is A |
| | NO:70) | | | | or absent |
| 17 | IEEQ | KTA | DKANHE | EQA | YQSAXaa₁Xaa₂ (SEQ ID |
| | (SEQ | | (SEQ ID NO: | | NO: 63), wherein Xaa₁ is L, |
| | ID NO: | | 62) | | A, or absent, and Xaa₂ is A |
| | 24) | | | | or absent |
| 18 | IEEQ | KEA | DKANHE | EQA | YQSAXaa₁Xaa₂ (SEQ ID |
| | (SEQ | | (SEQ ID NO: | | NO: 63), wherein Xaa₁ is L, |
| | ID NO: | | 62) | | A, or absent, and Xaa₂ is A |
| | 24) | | | | or absent |

In certain instances, the sequences set forth above in Table 5 can have at least one (e.g., 1, 2, 3, 4, 5, or 6) amino acid substitution or deletion. The ACE2 α11 helix peptides can include any amino acid sequence described herein.

In addition to the sequences provided in Table 5, peptides comprising Formula (II) also include SEQ TD NOs: 115, 116, 120, 121, 126, and 128-133, as shown in FIG. 8.

In some instances, a peptide comprising Formula (II) comprising the sequences set forth above in Table 5 can have one or more of the properties listed below: (i) the peptide prevents or inhibits the interaction between Angiotensin converting enzyme 2 (ACE2) and a virus whose receptor binding domain binds ACE2; (ii) the peptide is alpha helical; (iii) the peptide is protease resistant; and/or (iv) the peptide blocks or inhibits infection of human ACE2 expressing epithelial cells. In some instances, the epithelial cells are located in the respiratory system.

The tether of Formula (II) can include an alkyl, alkenyl, or alkynyl moiety (e.g., C₅, C₈, C₁₁, or C₁₂ alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances of Formula (II), x is 2, 3, or 6. In some instances of Formula (II), each y is independently an integer between 0 and 15, or 3 and 15. In some instances of Formula (II), R₁ and R₂ are each independently H or C₁-C₆ alkyl. In some instances of Formula (II), R₁ and R₂ are each independently C₁-C₃ alkyl. In some instances of Formula (II), at least one of R₁ and R₂ are methyl. For example, R₁ and R₂ can both be methyl. In some instances of Formula (II), R₃ is alkyl (e.g., C₈ alkyl) and x is 3. In some instances of Formula (II), R₃ is C₁₁ alkyl and x is 6. In some instances of Formula (II), R₃ is alkenyl (e.g., C₈ alkenyl) and x is 3. In some instances of Formula (II), x is 6 and R₃ is C₁₁ alkenyl. In some instances, R₃ is a straight chain alkyl, alkenyl, or alkynyl. In some instances, R₃ is CH₂—CH₂—CH₂—CH═CH—CH₂—CH₂—CH₂.

In another aspect, disclosed herein is a compound comprising a stabilized peptide comprising a sequence having the formula:

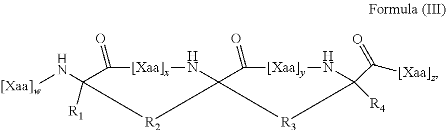

or a pharmaceutically acceptable salt thereof. In some instances, R₁ and R₄ is independently H, alkyl, alkenyl, alkynyl, arylalkyl, cycloalkylalkyl, heteroarylalkyl, or heterocyclylalkyl, any of which is substituted or unsubstituted. In some instances, each R₂ and R₃ is independently alkylene, alkenylene, or alkynylene, any of which is substituted or unsubstituted. In some instances, each u and x is independently 2, 3, or 6. In some instances, each t, v, and y is independently 1, 2, 3, 4, 5, 6, 7, 8, 9, 10, 11, 12, 13, 14, 15, 16, 17, 18, 19, 20, 21, 22, or 23. In some instances, each Xaa is independently an amino acid. In some instances, the stabilized peptide prevents or inhibits the interaction between Angiotensin converting enzyme 2 (ACE2) and a virus whose receptor binding domain binds ACE2. In some instances, the sequence has SEQ ID NO: 1 with:
(i) at least 3 amino acids that are substituted with α, α-disubstituted non-natural amino acids with olefinic side chains that form an internal cross-link;
(ii) 0, 1, 2, 3, 4, 5, 6, 7, 8, 9, 10, or 11 additional amino acid substitutions, and
(iii) 0, 1, 2, 3, 4, or 5 amino acid deletions at the N and/or C-terminus of the sequence.

In some instances, each R₁ and R₄ is independently H or a C₁₋₁₀ alkyl, alkenyl, alkynyl, arylalkyl, cycloalkylalkyl, heteroarylalkyl, or heterocyclylalkyl, any of which is substituted or unsubstituted. In some instances, each of R₂ and R₃ is independently a C₅₋₂₀ alkyl, alkenyl, alkynyl; [R₄—K—R₄]ₙ; each of which is substituted with 0-6 R₅. In some instances, R₅ is halo, alkyl, OR₆, N(R₆)₂, SR₆, SOR₆, SO₂R₆, CO₂R₆, R₆, a fluorescent moiety, or a radioisotope; K is O, S, SO, SO₂, CO, CO₂, CONR₆, or

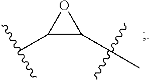 [SMILES: *C1OC1*]

In some instances, R₆ is H, alkyl, or a therapeutic agent. In some instances, n is an integer from 1-4. In some instances, [Xaa]_{w}; [Xaa]ₓ; [Xaa]_{y}; and [Xaa]z are provided in Table 5.

In some instances, a peptide comprising Formula (III) can have one or more of the properties listed below: (i) the peptide prevents or inhibits the interaction between Angiotensin converting enzyme 2 (ACE2) and a virus whose receptor binding domain binds ACE2; (ii) the peptide is alpha helical; (iii) the peptide is protease resistant; and/or (iv) the peptide blocks or inhibits infection of human ACE2 expressing epithelial cells. In some instances, the epithelial cells are located in the respiratory system.

The tether of Formula (III) can include an alkyl, alkenyl, or alkynyl moiety (e.g., C₅, C₈, C₁₁, or C₁₂ alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances of Formula (III), x is 2, 3, or 6. In some instances of Formula (III), each y is independently an integer between 0 and 15, or 3 and 15. In some instances of Formula (III), R₁ and R₂ are each independently H or C₁-C₆ alkyl. In some instances of Formula (III), R₁ and R₂ are each independently C₁-C₃ alkyl. In some instances of Formula (III), at least one of R₁ and R₂ are methyl. For example, R₁ and R₂ can both be methyl. In some instances of Formula (III), R₃ is alkyl (e.g., C₈ alkyl) and x is 3. In some instances of Formula (III), R₃ is C₁₁ alkyl and x is 6. In some instances of Formula (III), R₃ is alkenyl (e.g., C₈ alkenyl) and x is 3. In some instances of Formula (III), x is 6 and R₃ is C₁₁ alkenyl. In some instances, R₃ is a straight chain alkyl, alkenyl, or alkynyl. In some instances, R₃ is —CH₂—CH₂—CH₂—CH═CH—CH₂—CH₂—CH₂—.

In another aspect of Formula (III), of the three alpha, alpha disubstituted stereocenters: (i) two stereocenters are in the R configuration and one stereocenter is in the S configuration; or (ii) two stereocenters are in the S configuration and one stereocenter is in the R configuration. Thus, where Formula (III) is depicted as:

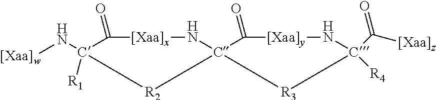

The C′ and C″′ disubstituted stereocenters can both be in the R configuration or they can both be in the S configuration. When both C′ and C″′ are in the R configuration, C″ is in the S configuration. When both C′ and C″′ are in the S configuration, C″ is in the R configuration. The double bond in each of R₂ and R₃ of Formula (III) can be in the E or Z stereochemical configuration.

In some instances of Formula (III), R₃ is [R₄—K—R₄]ₙ; and R₄ is a straight chain alkyl, alkenyl, or alkynyl.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, the term “cycloalkylalkyl,” employed alone or in combination with other terms, refers to a group of formula cycloalkyl-alkyl-. In some embodiments, the alkyl portion has 1 to 4, 1 to 3, 1 to 2, or 1 carbon atom(s). In some embodiments, the alkyl portion is methylene. In some embodiments, the cycloalkyl portion has 3 to ring members or 3 to 7 ring members. In some embodiments, the cycloalkyl group is monocyclic or bicyclic. In some embodiments, the cycloalkyl portion is monocyclic. In some embodiments, the cycloalkyl portion is a C₃-7 monocyclic cycloalkyl group.

As used herein, the term “heteroarylalkyl,” employed alone or in combination with other terms, refers to a group of formula heteroaryl-alkyl-. In some embodiments, the alkyl portion has 1 to 4, 1 to 3, 1 to 2, or 1 carbon atom(s). In some embodiments, the alkyl portion is methylene. In some embodiments, the heteroaryl portion is a monocyclic or bicyclic group having 1, 2, 3, or 4 heteroatoms independently selected from nitrogen, sulfur and oxygen. In some embodiments, the heteroaryl portion has 5 to carbon atoms.

As used herein, the term “substituted” means that a hydrogen atom is replaced by a non-hydrogen group. It is to be understood that substitution at a given atom is limited by valency.

As used herein, “halo” or “halogen”, employed alone or in combination with other terms, includes fluoro, chloro, bromo, and iodo. In some embodiments, halo is F or Cl.

In some embodiments, the disclosure features structurally-stabilized (e.g., stapled or stitched) peptides comprising the amino acid sequence of any one of SEQ ID NO: 1 or 21 (or a modified version thereof), wherein: the side chains of two amino acids separated by two, three, or six amino acids are replaced by an internal staple, the side chains of three amino acids are replaced by an internal stitch, the side chains of four amino acids are replaced by two internal staples, or the side chains of five amino acids are replaced by the combination of an internal staple and an internal stitch. In some embodiments, the disclosure features structurally-stabilized (e.g., stapled or stitched) peptides comprising the amino acid sequence of any one of SEQ ID NO: 1 or 21 (or a modified version thereof), wherein the side chains of two amino acids separated by two, three, or six amino acids are replaced by an internal staple. In some embodiments, the disclosure features structurally-stabilized (e.g., stapled or stitched) peptides comprising the amino acid sequence of any one of SEQ ID NO: 1 or 21 (or a modified version thereof), wherein the side chains of two amino acids separated by three amino acids are replaced by an internal staple. In some embodiments, the disclosure features structurally-stabilized (e.g., stapled or stitched) peptides comprising the amino acid sequence of any one of SEQ ID NO: 1 or 21 (or a modified version thereof), wherein the side chains of two amino acids separated by six amino acids are replaced by an internal staple. In some embodiments, the disclosure features structurally-stabilized (e.g., stapled or stitched) peptides comprising the amino acid sequence of any one of SEQ ID NO: 1 or 21 (or a modified version thereof), wherein the side chains of three amino acids are replaced by an internal stitch.

The stapled or stitched peptide can be 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, or 100 amino acids in length. In one instance, the stapled or stitched peptide is 20-25, 20-30, 20-35, 20-40, 20-45, 20-45, 20-50, 20-60, 20-70, 20-80, 20-85, 20-90, 20-95, or 20-100 amino acids in length. In a specific embodiment, the stapled or stitched peptide is 24-45 amino acids (i.e., 24, 25, 26, 27, 28, 29, 30, 31, 32, 33, 34, 35, 36, 37, 38, 39, 40, 41, 42, 43, 44, 45) in length. In a specific embodiment, the stapled or stitched peptide is 24-42 amino acids (i.e., 24, 25, 26, 27, 28, 29, 30, 31, 32, 33, 34, 35, 36, 37, 38, 39, 40, 41, 42) amino acids in length. In a specific embodiment, the stapled or stitched peptide is 24-35 amino acids (i.e., 24, 25, 26, 27, 28, 29, 30, 31, 32, 33, 34, 35) amino acids in length. In a specific embodiment, the stapled or stitched peptide is 24 amino acids in length. In a specific embodiment, the stapled or stitched peptide is 26 amino acids in length. In another specific embodiment, the stapled or stitched peptide is 35 amino acids in length. In another specific embodiment, the stapled or stitched peptide is 40 amino acids in length. In another specific embodiment, the stapled or stitched peptide is 45 amino acids in length. In another specific embodiment, the stapled or stitched peptide is 50 amino acids in length. Exemplary ACE2 α1 helix stapled or stitched peptides are shown in Tables 3-5 and described in Formulae (I)-(III). In one embodiment, the ACE2 α1 helix stapled or stitched peptide comprises or consists of a stapled or stitched version of the amino acid sequence of any one of SEQ ID NOs: 1, 13-16, 21, or 145-148 (e.g., the product of a ring-closing metathesis reaction performed on a peptide comprising the amino acid sequence of any one of SEQ ID NOs: 1, 13-16, 21, or 145-148, respectively). In one embodiment, the ACE2 α1 helix stapled or stitched peptide comprises or consists of a stapled or stitched version of the amino acid sequence of SEQ ID NO: 1 (e.g., the product of a ring-closing metathesis reaction performed on a peptide comprising the amino acid sequence of SEQ ID NO:1). In one embodiment, the ACE2 α1 helix stapled or stitched peptide comprises or consists of a stapled or stitched version of the amino acid sequence of SEQ ID NO: 21 (e.g., the product of a ring-closing metathesis reaction performed on a peptide comprising the amino acid sequence of SEQ ID NO: 21).

In certain embodiments, the stapled peptide comprises or consists of a variant of the amino acid sequence set forth in any one of SEQ ID NO: 1 or 21, wherein two amino acids each separated by 3 amino acids (i.e., positions i and i+4) are modified to structurally stabilize the peptide (e.g., by substituting them with non-natural amino acids to permit hydrocarbon stitching, i.e., stapling amino acids). In certain embodiments, the stapled peptide comprises or consists of a variant of the amino acid sequence set forth in any one of SEQ ID NO: 1 or 21, wherein two amino acids each separated by 6 amino acids (i.e., positions i and i+7) are modified to structurally stabilize the peptide (e.g., by substituting them with non-natural amino acids to permit hydrocarbon stapling, i.e., with stapling amino acids).

While hydrocarbon tethers are common, other tethers can also be employed in the structurally-stabilized ACE2 α1 helix peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the ACE2 α1 helix peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc.,113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U.S.A, 97:3803-3808 (2000); Double-click stapling: Lau et al., Chem. Sci., 5:1804-1809 (2014); Bis-lactam stapling: J. C. Phelan et al., J. Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+3; i to i+4; and i to i+7 are common in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

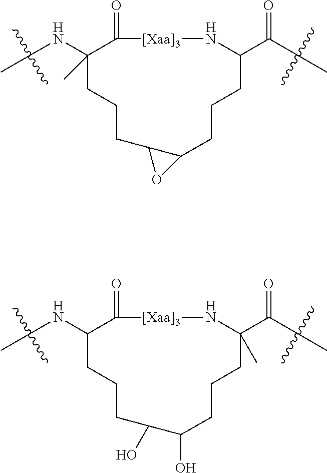

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the peptide or via the amino acid side chain. Other agents can be attached to the functionalized tether, e.g., an agent that facilitates entry of the peptide into cells.

In some instances, alpha disubstituted amino acids are used in the peptide to improve the stability of the alpha helical secondary structure. However, alpha disubstituted amino acids are not required, and instances using mono-alpha substituents (e.g., in the tethered amino acids) are also envisioned.

The structurally-stabilized (e.g., stapled or stitched) peptides can include a drug, a toxin, a derivative of polyethylene glycol; a second peptide; a carbohydrate, etc. Where a polymer or other agent is linked to the structurally-stabilized (e.g., stapled or stitched) peptide, it can be desirable for the composition to be substantially homogeneous.

The addition of polyethelene glycol (PEG) molecules can improve the pharmacokinetic and pharmacodynamic properties of the peptide. For example, PEGylation can reduce renal clearance and can result in a more stable plasma concentration. PEG is a water soluble polymer and can be represented as linked to the peptide as formula:
XO—(CH₂CH₂O)ₙ—CH₂CH₂—Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the peptide. Y may also be a maleimide linkage to a thiol group (including but not limited to, the thiol group of cysteine). Other methods for linking PEG to a peptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain embodiments, macromolecular polymer (e.g., PEG) is attached to a structurally-stabilized (e.g., stapled or stitched) peptide described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

The structurally-stabilized (e.g., stapled or stitched) peptides can also be modified, e.g., to further facilitate cellular uptake or increase in vivo stability, in some embodiments. For example, acylating or PEGylating a structurally-stabilized peptide facilitates cellular uptake, increases bioavailability, increases blood circulation, alters pharmacokinetics, decreases immunogenicity and/or decreases the needed frequency of administration.

In some embodiments, the structurally-stabilized (e.g., stapled or stitched) peptides disclosed herein have an enhanced ability to penetrate cell membranes (e.g., relative to non-stabilized peptides). See, e.g., International Publication No. WO 2017/147283, which is incorporated by reference herein in its entirety.

### Conjugates to the Polypeptides or Stabilized Peptides

Disclosed herein are conjugates that associate with (e.g., are conjugated to) a peptide or stabilized peptide disclosed herein. Conjugates are moieties that are used to isolate and/or detect a peptide disclosed herein. In some instances, the moiety increased the half-life of the peptide to which it is conjugated. In some instances, the moiety is a detection moiety. In some instances, the moiety is a capture moiety such as biotin. In some instances, the peptide is conjugated to a bead. In some instances, the bead is magnetic.

In some instances, the conjugate functions to extend the half-life of the peptide. In some instances, the peptide is conjugated to a moiety that increases the overall size of the peptide molecule. In some instances, the moiety is a polyethylene glycol (PEG). In some instances, other moieties known in the art are conjugated to a peptide disclosed herein. For example, in one aspect, human serum albumin (HSA) is conjugated to a peptide disclosed herein.

In some instances, a detection moiety is conjugated to a peptide disclosed herein. In some instances, the detection moiety is a fluorescent, radioactive, chemiluminescent, or colorimetric detectable markers. Any suitable detectable label can be used. In some embodiments, the detectable label is a fluorophore (e.g., GFP, FITC, Cy5, etc.). In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate chemical substrates (e.g., an oxidizing reagent plus a chemiluminescent compound). A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters.

In some instances, the peptide is conjugated to a capture moiety. A capture moiety can be used for purification or capture of the peptide of interest. For example, in some instances, the capture moiety is a biotin molecule. In this setting, an avidin or streptavidin molecule (e.g., on a support) can be used to capture the biotin-conjugated peptide. In some instances, the capture moiety is a fusion protein tag. In some instances, the fusion protein tag includes His, HA, Flu, or FLAG tag.

### Pharmaceutical Compositions

One or more of any of the structurally-stabilized (e.g., stapled or stitched) peptides described herein can be formulated for use as or in pharmaceutical compositions. The pharmaceutical compositions may be used in the methods of treatment or prevention described herein (see above). In certain embodiments, the pharmaceutical composition comprises a structurally-stabilized (e.g., stapled or stitched) peptide comprising or consisting of an amino acid sequence that is identical to an amino acid sequence set forth in Table 1, except for 1 to 10, 1 to 9, 1 to 8, 1 to 7, 1 to 6, 1 to 5, 1 to 4, 1 to 3, 1 to 2, or 1 amino acid substitution, insertion, or deletion. These changes to the amino acid sequences can be made on the non-interacting alpha-helical face of these peptides (i.e., to the amino acids that do not interact with the coronavirus 5 helix bundle) and/or on the interacting alpha-helical face (i.e., to the amino acids that interact with the coronavirus 5 helix bundle). Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal administration, and/or topical administration (including topical (e.g., nasal) sprays and/or solutions).

In some instances, pharmaceutical compositions can include an effective amount of one or more structurally-stabilized (e.g., stapled or stitched) peptides. The terms “effective amount” and “effective to treat,” as used herein, refer to an amount or a concentration of one or more structurally-stabilized (e.g., stapled or stitched) peptides or a pharmaceutical composition described herein utilized for a period of time (including acute or chronic administration and periodic or continuous administration) that is effective within the context of its administration for causing an intended effect or physiological outcome (e.g., treatment of infection).

Pharmaceutical compositions of this invention can include one or more structurally-stabilized (e.g., stapled or stitched) peptides described herein and any pharmaceutically acceptable carrier and/or vehicle. In some instances, pharmaceuticals can further include one or more additional therapeutic agents in amounts effective for achieving a modulation of disease or disease symptoms.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

In some instances, one or more structurally-stabilized (e.g., stapled or stitched) peptides disclosed herein can be conjugated, for example, to a carrier protein. Such conjugated compositions can be monovalent or multivalent. For example, conjugated compositions can include one structurally-stabilized (e.g., stapled or stitched) peptide disclosed herein conjugated to a carrier protein. Alternatively, conjugated compositions can include two or more structurally-stabilized (e.g., stapled or stitched) peptides disclosed herein conjugated to a carrier.

As used herein, when two entities are “conjugated” to one another they are linked by a direct or indirect covalent or non-covalent interaction. In certain embodiments, the association is covalent. In other embodiments, the association is non-covalent. Non-covalent interactions include hydrogen bonding, van der Waals interactions, hydrophobic interactions, magnetic interactions, electrostatic interactions, etc. An indirect covalent interaction occurs when two entities are covalently connected, optionally through a linker group.

Carrier proteins can include any protein that increases or enhances immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561, 1991; Szu et al., J. Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

### Methods of Treatment

The disclosure features methods of using any of the structurally-stabilized (e.g., stapled or stitched) peptides (or pharmaceutical compositions comprising said structurally-stabilized peptides) described herein for the prevention and/or treatment of a coronavirus (e.g., betacoronavirus) infection or coronavirus disease. The terms “treat” or “treating,” as used herein, refers to alleviating, inhibiting, or ameliorating the disease or infection from which the subject (e.g., human) is suffering. In some instances, the subject is an animal. In some embodiments, the subject is a mammal such as a non-primate (e.g., cow, pig, horse, cat, dog, rat, etc.) or a primate (e.g., monkey or human). In some instances, the subject is a domesticated animal (e.g., a dog or cat). In some instances, the subject is a bat. In some instances, the subject is a human. In certain embodiments, such terms refer to a non-human animal (e.g., a non-human animal such as a pig, horse, cow, cat or dog). In some embodiments, such terms refer to a pet or farm animal. In some embodiments, such terms refer to a human.

The structurally-stabilized (e.g., stapled or stitched) peptides (or compositions comprising the peptides) described herein can be useful for treating a subject (e.g., human subject) having a coronavirus (e.g., betacoronavirus) infection. The structurally-stabilized (e.g., stapled or stitched) peptides (or compositions comprising the peptides) described herein can also be useful for treating a human subject having a coronavirus disease. In certain embodiments, the coronavirus infection is an infection of one of 229E (alphacoronavirus); NL63 (alphacoronavirus); OC43 (betacoronavirus); HKU1 (betacoronavirus); Middle East respiratory syndrome (MERS); SARS-CoV-1; or SARS-CoV-2. In certain embodiments, the coronavirus disease is caused by a SARS-CoV-2 infection.

The structurally-stabilized (e.g., stapled or stitched) peptides (or compositions comprising the peptides) described herein can be useful for preventing (i.e., prophylaxis treatment of) a coronavirus (e.g., betacoronavirus) infection in a subject. The peptides (or compositions comprising the peptides) described herein can also be useful for preventing a coronavirus disease in a subject (e.g., human subject). In certain embodiments, the coronavirus infection is an infection of one of 229E (alphacoronavirus); NL63 (alphacoronavirus); OC43 (betacoronavirus); HKU1 (betacoronavirus); Middle East respiratory syndrome (MERS); SARS-CoV-1; or SARS-CoV-2. In certain embodiments, the coronavirus disease is caused by a SARS-CoV-2 infection.

The structurally-stabilized (e.g., stapled or stitched) peptides (or compositions comprising the peptides) described herein can also be useful for treating a subject with post-acute sequelae of SARS-CoV-2 infection.

In addition, the structurally-stabilized (e.g., stapled or stitched) peptides (or compositions comprising the peptides) described herein can also be useful for treating or preventing infection by a SARS-CoV-2 variant in a subject.

Also provided are methods of preventing or inhibiting interaction between the receptor binding domain of a virus and ACE2 in a subject in need thereof using the structurally-stabilized (e.g., stapled or stitched) peptides (or compositions comprising the peptides) described herein. In some cases, the virus can be a coronavirus (e.g., SARS-CoV-1 or SARS-CoV-2).

In certain embodiments, the subject in need thereof is administered a peptide described in Tables 1-5, or a variant thereof. In certain embodiments, the human subject in need thereof is administered a stapled ACE2 α1 helix peptide comprising or consisting of SEQ ID NO: 1 or a modified version (variant) thereof. In certain embodiments, the human subject in need thereof is administered a stapled ACE2 α1 helix peptide comprising or consisting of SEQ ID NO:21 or a modified version (variant) thereof.

In certain embodiments, the subject in need thereof is administered any one of the peptides having SEQ ID NOs: 1, 13-16, 21, 76, 77, or 145-148 described in Table 1, or a peptide of SEQ ID NOs. 112, 113, 117, 118, 123, 125, or 127, or a variant of any of these peptides that can still bind the S1 protein and/or RBD of SARS-CoV-2 or of a SARS-CoV-2 variant. In certain embodiments, the human subject in need thereof is administered any one of the peptides described in Table 3 or FIG. 8, or a variant thereof that can still bind the S1 protein and/or RBD of SARS-CoV-2 or of a SARS-CoV-2 variant. In certain embodiments, the human subject in need thereof is administered any one of the peptides described in FIG. 8. In one instance, the human subject in need thereof is administered any one of the peptides set forth in SEQ ID NOs.: 90-95, 98-100, 105-108, 110, 115, 116, 120, 121, 126, 130, 131, 132, or 133. In some embodiments, the human subject is infected with a coronavirus (e.g., betacoronavirus). In some embodiments, the human subject is at risk of being infected with a coronavirus (e.g., betacoronavirus). In some embodiments, the human subject is at risk of developing a coronavirus disease (e.g., betacoronavirus). In some instances, a human subject is at risk of being infected with a coronavirus or at risk of developing a coronavirus disease if he or she lives in an area (e.g., city, state, country) subject to an active coronavirus outbreak (e.g., an area where at least 1, at least 2, at least 3, at least 4, at least 5, at least 6, at least 7, at least 8, at least 9, at least 10, at least 20, at least 30, at least 40, or more people have been diagnosed as infected with a coronavirus). In some instances, a human subject is at risk of being infected with a coronavirus or developing a coronavirus disease if he or she lives in an area near (e.g., a bordering city, state, country) a second area (e.g., city, state, country) subject to an active coronavirus outbreak (e.g., an area near (e.g., bordering) a second area where at least 1, at least 2, at least 3, at least 4, at least 5, at least 6, at least 7, at least 8, at least 9, at least 10, at least 20, at least 30, at least 40, or more people have been diagnosed as infected with a coronavirus). In certain embodiments, the coronavirus disease is caused by a SARS-CoV-2 infection.

In some instances, also disclosed are methods of treatment or prevention that include a combination therapy. In some instances, the combination therapy treats or prevents a SARS virus infection (e.g., SARS-CoV-2 or a SARS-CoV-2 variant). In some instances, the combination therapy comprises any one of the polypeptides in FIG. 8 or Table 3. In some instances, the combination therapy includes a pharmaceutical composition that comprises any one of the polypeptides in FIG. 8 or Table 3. In some instances, the combination therapy further includes one or more of: dexamethasone, remdesivir, baricitinib in combination with remdesivir, favipiravir, merimepodib, an anticoagulation drug selected from low-dose heparin or enoxaparin, bamlanivimab, a combination of bamlanivimab and etesevimab, a combination of casirivimab and imdevimab, convalescent plasma, an mRNA SARS-CoV-2 vaccine (such as those produced by Moderna or Pfizer), an attenuated SARS-CoV-2 virus vaccine, or a dead SARS-CoV-2 virus vaccine. In some instances, the combination therapy comprises a viral vaccine against SARS-CoV-2 (e.g., an adenovirus vaccine such as those produced by Astra Zeneca and Johnson & Johnson. In some instances, the combination therapy comprises a monoclonal antibody that binds the coronavirus (e.g., SARS-CoV-2) and inhibits infection of a human subject. In some instances, the combination therapy comprises orthogonal entry inhibitors, such as antibodies, peptides, and small molecules; and furin inhibitors such as decanoyl-RVKR-chloromethylketone (CMK) and naphthofluorescein. See e.g., the agents described in Huang et al., Acta Pharmacologica Sinica (2020) 41:1141-1149; https://doi.org/10,1038/s41401-020-0485-4, which is incorporated by reference herein. In some instances, the combination therapy is with any one or more of a stabilized peptide(s) described in PCT/US2021/020940 (which is incorporated by reference herein).

In general, methods include selecting a subject and administering to the subject an effective amount of one or more of the structurally-stabilized (e.g., stapled or stitched) peptides herein, e.g., in or as a pharmaceutical composition, and optionally repeating administration as required for the prevention or treatment of a coronavirus infection or a coronavirus disease and can be administered intranasally (e.g. nose spray), as an inhalant (e.g. nebulization to access the respiratory system), orally, intravenously or topically. A subject can be selected for treatment based on, e.g., determining that the subject has a coronavirus (e.g., betacoronavirus) infection. The peptides of this disclosure can be used to determine if a subject's is infected with a coronavirus.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

### Methods of Diagnosis

Provided herein is a method of diagnosing a subject as having an infection caused by SARS-CoV-2 using a structurally-stabilized (e.g., stapled) peptide described herein.

In some instances, the peptides used for methods of diagnosis include SEQ ID NOs: 1, 13-16, 21, or 145-148 as shown in Table 1. In some embodiments, the peptides used for methods of diagnosis include SEQ ID NOs: 2-12 17-20, 134-143, and 172 as shown in Table 3. In some instances, the peptides used for methods of diagnosis include SEQ ID NOs: 49-53, as shown in Table 6.

The disclosure features methods of using a structurally-stabilized peptide (e.g., any of the structurally-stabilized (e.g., stapled) peptides (or pharmaceutical compositions comprising said structurally-stabilized peptides) described herein) to determine whether a subject would be receptive to therapy using structurally-stabilized (e.g., stapled) peptide described herein.

Moreover, the disclosure additionally provides a method for predicting the efficacy of treatment using a structurally-stabilized (e.g., stapled) peptide described herein for a subject having an infection caused by SARS-CoV-2. In some instances, the methods include testing a cell of a subject having an infection caused by SARS-CoV-2 for the presence of SARS-CoV-2, and predicting that a structurally-stabilized (e.g., stapled) peptide would likely inhibit SARS-CoV-2 infection.

In some instances, the methods include isolating secretions (e.g., mucous, sputum, saliva) or cells (e.g., biopsy; e.g., liquid biopsy) from a subject having an infection caused by SARS-CoV-2. In some instances, the isolated cells are cultured and treated with one or more of the structurally-stabilized (e.g., stapled) peptides described herein.

In some embodiments, the methods can include developing a personalized treatment regimen for a subject having an infection caused by SARS-CoV-2. Such methods can include, e.g., identifying a subject with secretions or cells containing the virus that are sensitive to one or more structurally-stabilized (e.g., stapled) peptides as disclosed herein and treating the subject with one or more structurally-stabilized (e.g., stapled) peptides as disclosed herein. In some embodiments, the methods can include determining the most appropriate treatment for a subject having an infection caused by SARS-CoV-2.

In some instances, the method of detecting the presence of a virus (e.g., SARS-CoV-2) whose receptor binding domain causes infection by binding to ACE2 includes isolating a sample from a subject and providing the sample to a plurality of stabilized peptides as disclosed herein. In some instances, the stabilized peptides in this method include stabilized peptides that are conjugated to a detection moiety and stabilized peptides that are conjugated to a capture moiety. After mixing the sample with the two different types of conjugated peptides, the sample is provided to a diagnostic such as a test strip. In some instances, the detection moiety (e.g., any of the detection moieties disclosed herein) can be identified.

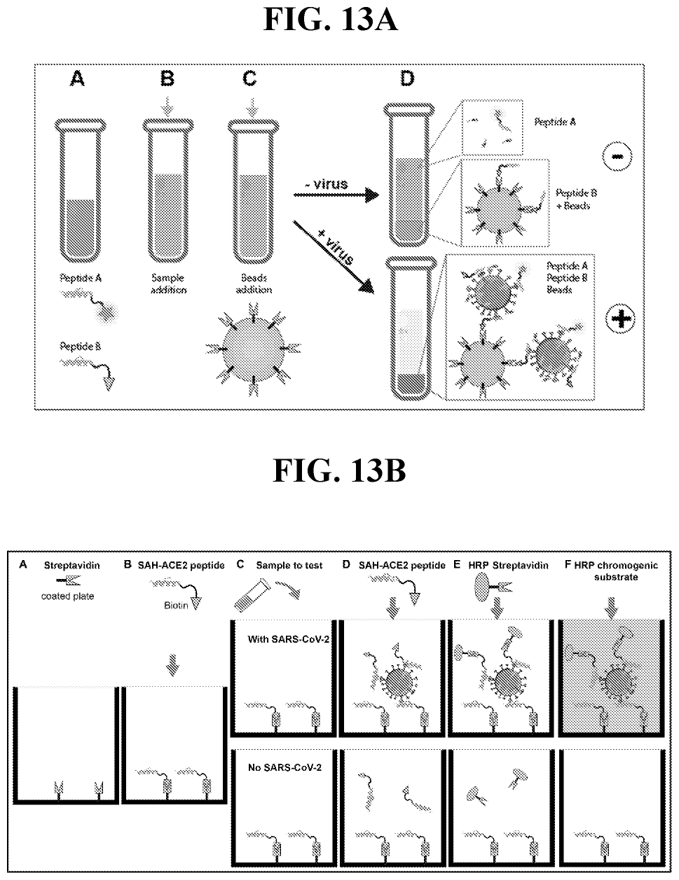

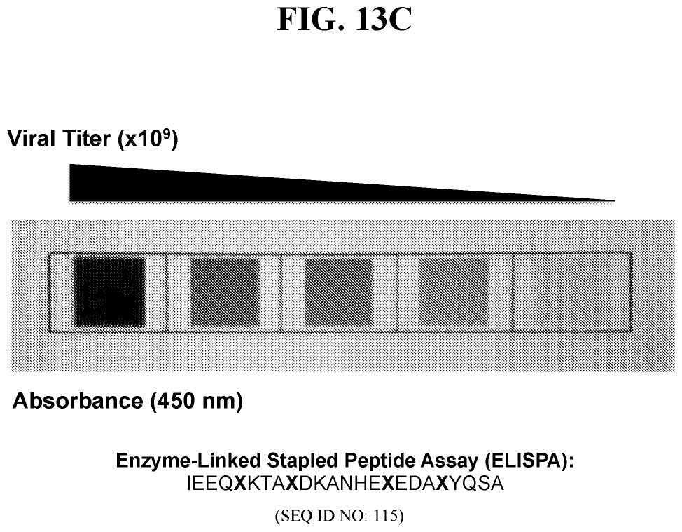

In some instances, the method of diagnosis is a method described in FIG. 13A or FIG. 13B or a modification thereof.

In some instances, the subject can be an animal. In some embodiments, the subject is a mammal such as a non-primate (e.g., cow, pig, horse, cat, dog, rat, etc.) or a primate (e.g., monkey or human). In some instances, the subject is a domesticated animal (e.g., a dog or cat). In some instances, the subject is a bat. In some instances, the subject is a human. In some instances, the subject is a human. In certain embodiments, such terms refer to a non-human animal (e.g., a non-human animal such as a pig, horse, cow, cat or dog). In some embodiments, such terms refer to a pet or farm animal. In some embodiments, such terms refer to a human.

### Kits

Provided herein is a kit that is used to detect SARS-CoV-2 infection. In some instances, the kit includes a test strip. In some instances, the kit includes one or more stapled proteins disclosed herein that is conjugated to a detection moiety (e.g., a fluorophore, chromophore, HRP, etc.), In some instances, the kit includes one or more stapled peptide disclosed herein that is conjugated to an affinity moiety (e.g, biotin) that can be captured by a solid support using streptavidin beads. In some instances, the kit further includes a capture resin (e.g. streptavidin beads for a biotin affinity label, Nickel NTA beads for a His-tag affinity label, anti-FLAG beads for a FLAG tag affinity label, etc.). Once captured using the affinity moiety, the peptide can be imaged for the presence of the detection moiety. In some instances, the kit further includes instructions for identification of the presence of the detection moiety. A positive test is determined by the presence of the detection label, such as fluorescence, and a negative test is determined by the absence of the detection label, as measured by eye, spectrometer, or other detection instrument. Like a pregnancy strip test, this diagnostic method can be used as a simple, rapid, and point-of-care method that does not require complex infrastructure or expertise to diagnose the infection. Thus, home use is also envisioned.

Also provided herein are kits comprising one or more structurally-stabilized ACE2 α1 antiviral peptides described herein In some embodiments, provided herein is a pharmaceutical pack or kit comprising one or more containers filled with one or more of the ingredients of the pharmaceutical compositions described herein, such as one or more structurally-stabilized peptides provided herein. In some embodiments, the kits contain a pharmaceutical composition described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for human administration. Also provided herein are kits that can be used in the above methods

### Methods of Making Stapled or Stitched Peptides

Stapled peptide synthesis: Fmoc-based solid-phase peptide synthesis was used to synthesize stapled peptide fusion inhibitors in accordance with our reported methods for generating all-hydrocarbon stapled peptides. To achieve the various staple lengths, α-methyl, α-alkenyl amino acids were installed at i, i+4 positions using two S-pentenyl alanine residues (S5). For the stapling reaction, Grubbs 1st generation ruthenium catalyst dissolved in dichloroethane was added to the resin-bound peptides. To ensure maximal conversion, three to five rounds of stapling were performed. The peptides were then cleaved off of the resin using trifluoroacetic acid, precipitated using a hexane:ether (1:1) mixture, air dried, and purified by LC-MS. All peptides were quantified by amino acid analysis.

Stitched peptide synthesis: Methods of synthesizing the stitched peptides described herein are known in the art. Nevertheless, the following exemplary method may be used. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The peptides of this invention can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH2 protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Insertion of a stitching amino acid may be performed as described in, e.g., Young and Schultz, J Biol Chem. 2010 Apr. 9; 285(15): 11039-11044. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from, e.g., Advanced Chemtech or Symphony X. Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (O—CH₂ or CH₂-0); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The peptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C₁-C₂₀ straight or branched alkyl groups); fatty acid radicals; and combinations thereof. α, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al., Current Protocols in Chemical Biology, 2011). In some instances for peptides where an i linked to i+7, i+7 linked to i+14 stitch is used (four turns of the helix stabilized): one R-octenyl alanine (e.g., (R)-α-(7′-octenyl)alanine), one one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one bis-pentenyl glycine (e.g., α,α-Bis(4′-pentenyl)glycine), and one S-octenyl alanine (e.g., (S)-α-(7′-octenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. R-octenyl alanine is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin (see, e.g., WO 2010/148335).

Fmoc-protected α-amino acids (other than the olefinic amino acids N-Fmoc-α,α-Bis(4′-pentenyl)glycine, (S)—N-Fmoc-α-(4′-pentenyl)alanine, (R)—N-Fmoc-α-(7′-octenyl)alanine, (R)—N-Fmoc-α-(7′-octenyl)alanine, and (R)—N-Fmoc-α-(4′-pentenyl)alanine), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stitching, and purifying the peptides disclosed herein are also known in the art (see, e.g., Bird et. al., Methods in Enzymol., 446:369-386 (2008); Bird et al., Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some instances, the peptides are substantially free of non-stitched peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific instance, a 50%/50% DMSO/NMP solution is used. The solution may be incubated for a period of 1, 6, 12 or 24 hours, following which the resin may be washed, for example with dichloromethane or NMP. In one instance, the resin is washed with NMP. Shaking and bubbling an inert gas into the solution may be performed.

Properties of the stitched or stapled peptides of the disclosure can be assayed, for example, using the methods described below and in the Examples.

### Assays to Determine Characteristics and Effectiveness of Stabilized Peptides

Assays to Determine α-Helicity: Compounds are dissolved in an aqueous solution (e.g. 5 μM potassium phosphate solution at pH 7, or distilled H₂O, to concentrations of 25-50 μM). Circular dichroism (CD) spectra are obtained on a spectropolarimeter (e.g., Jasco J-710, Aviv) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol., 1986).

Assays to Determine Melting Temperature (Tm): Cross-linked or the unmodified template peptides are dissolved in distilled H₂O or other buffer or solvent (e.g. at a final concentration of 50 μM) and Tm is determined by measuring the change in ellipticity over a temperature range (e.g. 4 to 95° C.) on a spectropolarimeter (e.g., Jasco J-710, Aviv) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In Vitro Protease Resistance Assays: The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The peptidomimetic macrocycles of the present invention may be subjected to in vitro enzymatic proteolysis (e.g. trypsin, chymotrypsin, pepsin) to assess for any change in degradation rate compared to a corresponding uncrosslinked or alternatively stapled polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time.

Peptidomimetic macrocycles and/or a corresponding uncrosslinked polypeptide can be each incubated with fresh mouse, rat and/or human serum (e.g. 1-2 mL) at 37° C. for, e.g., 0, 1, 2, 4, 8, and 24 hours. Samples of differing macrocycle concentration may be prepared by serial dilution with serum. To determine the level of intact compound, the following procedure may be used: The samples are extracted, for example, by transferring 100 μL of sera to 2 ml centrifuge tubes followed by the addition of 10 μL of 50% formic acid and 500 μL acetonitrile and centrifugation at 14,000 RPM for 10 min at 4+/−2° C. The supernatants are then transferred to fresh 2 ml tubes and evaporated on Turbovap under N2<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

Plasma Stability Assay: Stapled peptide stability can be tested in freshly drawn mouse plasma collected in lithium heparin tubes. Triplicate incubations are set up with 500 μl of plasma spiked with 10 μM of the individual peptides. Samples are gently shaken in an orbital shaker at 37° C. and 25 μl aliquots are removed at 0, 5, 15, 30, 60, 240, 360 and 480 min and added to 100 μl of a mixture containing 10% methanol: 10% water: 80% acetonitrile to stop further degradation of the peptides. The samples are allowed to sit on ice for the duration of the assay and then transferred to a MultiScreen® Solvinert 0.45 μm low-binding hydrophilic PTFE plate (Millipore). The filtrate is directly analyzed by LC-MS/MS. The peptides are detected as double or triple charged ions using a Sciex 5500 mass spectrometer. The percentage of remaining peptide is determined by the decrease in chromatographic peak area and log transformed to calculate the half-life.

In Vivo Protease Resistance Assays: A key benefit of peptide stapling is the translation of in vitro protease resistance into markedly improved pharmacokinetics in vivo.

Liquid chromatography/mass spectrometry-based analytical assays are used to detect and quantitate SARS-CoV-2 levels in plasma. For pharmacokinetic analysis, peptides are dissolved in sterile aqueous 5% dextrose (1 mg/mL) and administered to C57BL/6 mice (Jackson Laboratory) by bolus tail vein or intraperitoneal injection (e.g. 5, 10, 25, 50 mg/kg). Blood is collected by retro-orbital puncture at 5, 30, 60, 120, and 240 minutes after dosing 5 animals at each time point. Plasma is harvested after centrifugation (2,500×g, 5 minutes, 4° C.) and stored at −70° C. until assayed. Peptide concentrations in plasma are determined by reversed-phase high performance liquid chromatography with electrospray ionization mass spectrometric detection (Aristoteli et al., Journal of Proteome Res., 2007; Walden et al., Analytical and Bioanalytical Chem., 2004). Study samples are assayed together with a series of 7 calibration standards of peptide in plasma at concentrations ranging from 1.0 to 50.0 μg/mL, drug-free plasma assayed with and without addition of an internal standard, and 3 quality control samples (e.g. 3.75, 15.0, and 45.0 μg/mL). Standard curves are constructed by plotting the analyte/internal standard chromatographic peak area ratio against the known drug concentration in each calibration standard. Linear least squares regression is performed with weighting in proportion to the reciprocal of the analyte concentration normalized to the number of calibration standards. Values of the slope and y-intercept of the best-fit line are used to calculate the drug concentration in study samples. Plasma concentration-time curves are analyzed by standard noncompartmental methods using WinNonlin Professional 5.0 software (Pharsight Corp., Cary, NC), yielding pharmacokinetic parameters such as initial and terminal phase plasma half-life, peak plasma levels, total plasma clearance, and apparent volume of distribution.

Persistence of stabilized ACE2 α1 helix peptides in the nasal mucosa after topical administration (i.e. nose drops) and in the respiratory mucosa after nebulization is examined in the context of pre- and post-infection blockade of viral fusion and dissemination. Mice are exposed to single ACE2 α1 helix peptide treatment by nose drop or nebulizer at a series of intervals preceding intranasal infection with SARS-CoV-2, and the duration of protection from mucosal infection (assessed histologically as described above) used to measure the relative mucosal stability and prophylactic efficacy of ACE2 α1 helix constructs.

In vitro SARS CoV-2 RBD Protein Binding Assays: To assess the binding and affinity of peptidomimetic macrocycles and peptidomimetic precursors to acceptor proteins, a fluorescence polarization assay (FPA) can be used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein, such as in this case, recombinant SARS-CoV-2 RBD protein or recombinant SARS-CoV-2 spike protein) emit higher levels of polarized fluorescence due to their slower rates of rotation as compared to fluorescent tracers attached to smaller molecules (e.g. FITC-labeled peptides that are free in solution). A positive control binding interaction between a FITC-labeled ACE2h1 peptide and SARS-CoV-2 protein can also be used to conduct competitive FPAs, in which non-fluorescently labeled peptides are incubated with the FITC-peptide/RBD complex to assess the differential capacity of alternate SAH-ACE2h1 peptides to compete with the FITC-peptide for protein binding. Another method for evaluating the binding and affinity of peptidomimetic macrocycles and peptidomimetic precursors to acceptor proteins involves anchoring the test peptides on a solid support, such as the use of biotinylated SAH-ACE2h1 peptides bound to streptavidin-coated tips in a biolayer interferometry assay, whereby the association and dissociation of RBD protein in solution to the peptide-coated tip is monitored and quantitated. Binding assays are performed with FITC-labeled ACE2h1 peptides for direct FPAs, non-fluorescent ACE2h1 peptides for competitive FPAs, and affinity tagged ACE2h1 peptides (such as biotinylated peptides) for peptide capture on a compatible BLI tip (e.g. streptavidin coated tip for capture of biotinylated peptides). Target proteins for analysis include recombinant SARS-CoV-2 RBD or spike proteins expressed in E coli or HEK293 cells with GST or His tags that are either cleaved after purification or retained for affinity capture (e.g. glutathione plates, Ni-NTA beads or plates, etc.).

In vitro SARS-CoV-2 RBD Bead Binding Assay: To monitor the association of FITC-labeled ACE2h1 peptides to RBD-coated beads, which can be used for qualitative and quantitative assessment of binding activity, His-tagged RBD protein (25 μg) or vehicle is incubated with Ni-NTA agarose beads (100 μL) in PBS for 30 min. The beads are then incubated with FITC-labeled peptide (10 μM) for 30 min, isolated by benchtop centrifugation (2000×g), resuspended in PBS for plating in 386-well plate format (10 L/well), and imaged using an Olympus wide-field epifluorescence microscope, a 63× LCPlanFL NA 0.7 objective and a CoolSNAP DYNO camera.

Antiviral Efficacy Assays: The efficiency of ACE2 α1 helix peptides in preventing and treating COVID-19 infection are evaluated in monolayer cell cultures. A viral detection platform has been developed for SARS-CoV-2 based on previous screens against Ebolaviruses (see, Anantpadma M. et al., Antimicrob Agents Chemother. 2016; 60(8):4471-81. Epub 2016/05/11. doi: 10.1128/AAC.00543-16. PubMed PMID: 27161622; PMCID: PMC4958205). Vero E6 cells plated in 384-well format are treated for 1 hour with a serial dilution of stapled peptides (e.g. 10-25 μM starting dose), performed in triplicate, followed by 4 hour challenge with SARS-CoV-2 to achieve control infection of 10-20% cells (the pre-determined optimal infectivity to assess the dynamic range of test compounds in the assay). Infected cells are then washed, fixed with 4% paraformaldehyde, rewashed in PBS, immune-stained with anti-SARS-CoV-2 nucleocapsid monoclonal antibody followed by anti-Ig secondary antibody (Alexa Fluor 488; Life Technologies), and cell bodies counterstained with HCS CellMask blue. Cells are imaged across the z-plane on a Nikon Ti Eclipse automated microscope, analyzed by CellProfiler software, and infection efficiency calculated by dividing infected by total cells. Control cytotoxicity assays are performed using Cell-Titer Glo (Promega) and LDH release (Roche) assays.

In an alternative approach, qPCR based viral detection is used in natively-susceptible human-derived Huh770 and Calu-371 cells that express ACE2, and also MatTek Life Sciences primary lung epithelial and alveolar cell models, infected with SARS-CoV-2 virus (e.g. USA-WA1/2020; Hongkon VM20001061). Cultured cells are treated for 1 hour with a serial dilution of stapled peptides followed by challenge with SARS-CoV-2. Culture supernatants are sampled, virus lysed in the presence of RNAse inhibitor, and RT and qPCR performed as described. See Suzuki et al. J Vis Exp. 2018(141). Epub 2018/11/20. doi: 10.3791/58407. CDC-validated BHQ quenched dye pair primers are purchased from IDT and genome equivalents calculated from Ct values.

In yet another approach, antiviral activity of ACE2 α1 helix stapled peptides are assessed using pseudotyped virus. The 293T-hsACE2 stable cell line (Cat #C-HA101) and the pseudotyped SARS-CoV-2 (Wuhan-Hu-1 strain) viral particles coated with the SARS-CoV-2 spike protein and carrying RNA coding for GFP (Cat #RVP-701G, Lot #CG-113A) reporters are used (Integral Molecular). The neutralization assay is carried out according to the manufacturers' protocols. In brief, 5 μL of a single dose of peptide (5 μM final dose) is incubated with 5 μL pseudotyped SARS-CoV-2-GFP for 1 hr at 37° C. in a 384 well black clear bottom plate followed by addition of 30 μL of 1,000 293T-hsACE2 cells in 10% FBS DMEM, phenol red free media and placed in a humidified incubator for 48 or 72 hrs. Hoechst 33342 and DRAQ7 dyes are added and the plate imaged on a Molecular Devices ImageXpress Micro Confocal Laser at 10× magnification. GFP (+) cells are counted and plotted using Prism software (Graphpad). To evaluate the capacity of lead stapled peptides to prevent SARS-CoV-2 infection, K18-hACE2 (Jackson Laboratory) mice (n=10 per arm; 5 male, 5 female) are administered intranasally or by the oropharyngeal route with stapled peptide or vehicle and 24 hours later a viral dosage of 10⁴ PFU is inoculated intranasally. Mice are euthanized 4 days later (peak of viremia) for evaluation by necropsy and viral load as quantitated by qPCR from supernatant samples of lung homogenates, prepared as described using a tissuelyzer (Qiagen). See Bao L et al. Nature. 2020. Epub 2020/05/08; doi: 10.1038/s41586-020-2312-y. To evaluate the capacity of lead stapled peptides to treat or mitigate established SARS-CoV-2 infection, K18-hACE2 mice (n=10 per arm; 5 male, 5 female) are inoculated intranasally at a viral dosage of 10⁴ PFU on day 1, followed by daily oropharyngeal or intraperitoneal treatment with stapled peptide or vehicle for 10 days (days 2-12). In an alternate design, dosing is delayed until 3-5 days post-inoculation to simulate symptom- or positive test-driven initiation of therapy. Mice are continuously monitored to record body weights and clinical signs, with disease progression scored as >10% body weight loss, labored breathing, and/or failure to thrive. Doses for the most effective compound and route are then be refined in both prevention and treatment studies to determine the minimum dose to protect mice. The same experimental design is used except that the 4 treatment groups (n=10; 5 male, 5 female) receive the original dose and then 3 progressively lowered doses in 4-fold increments.

Clinical Trials: To determine the suitability of the cross-linked polypeptides of the invention for treatment of humans, clinical trials can be performed. For example, patients exposed to SARS-CoV-2 infection or diagnosed with SARS-CoV-2 infection are selected and separated in treatment and one or more control groups, wherein the treatment group is administered a crosslinked polypeptide of the invention, while the control groups receive a placebo or a known antiviral drug. The treatment safety and efficacy of the cross-linked polypeptides of the invention can thus be evaluated by performing comparisons of the patient groups with respect to factors such as prevention of symptoms, time to resolution of symptoms, and/or overall infection severity. In another example, uninfected patients are identified and are given either a cross-linked polypeptide or a placebo. After receiving treatment, patients are followed. In both examples, the SARS-CoV-2-exposed patient group treated with a cross-linked polypeptide would avoid the development of infection, or a patient group with SARS-CoV-2 infection would show resolution of or relief from symptoms compared to a patient control group treated with a placebo.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Design and Synthesis of SAH-ACE2h1 Stapled Peptides

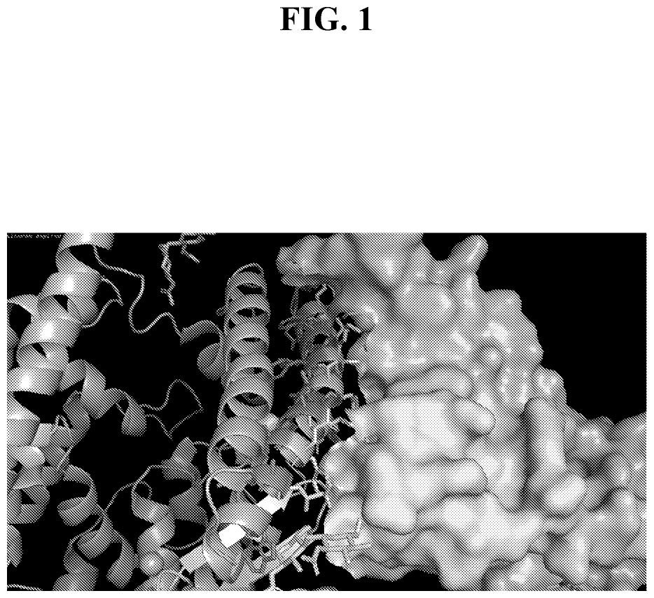

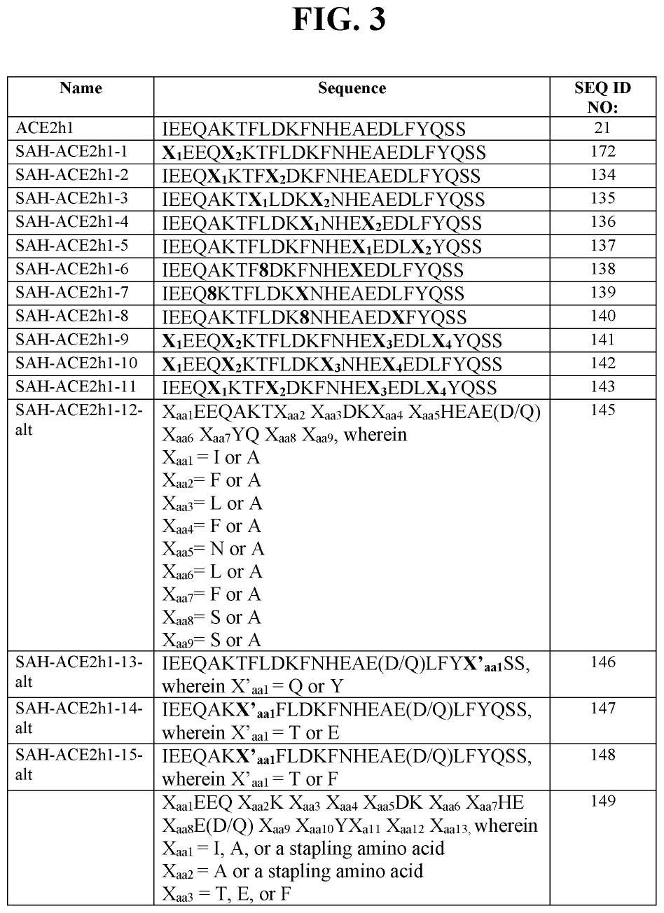

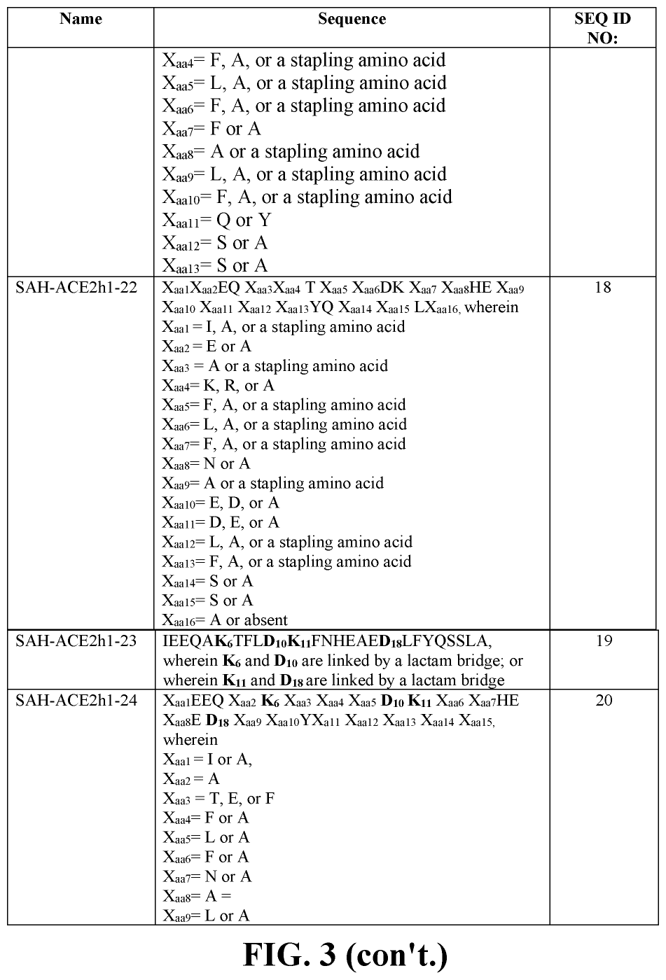

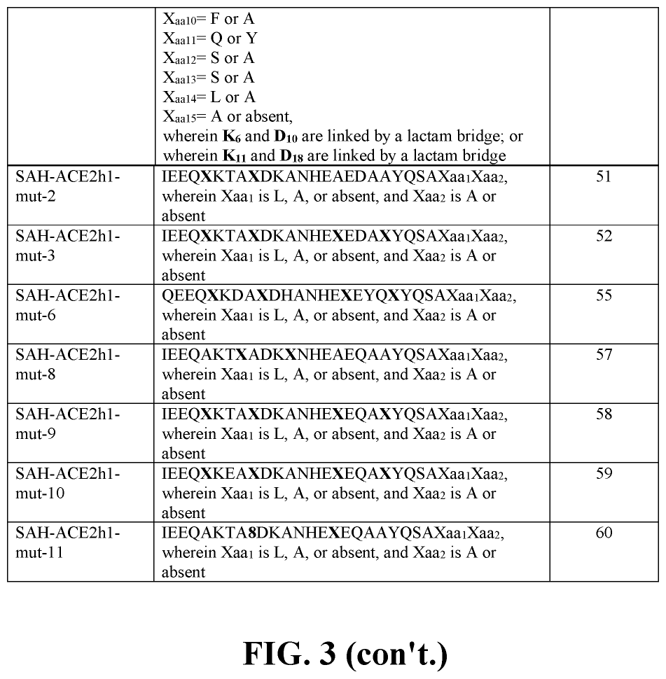

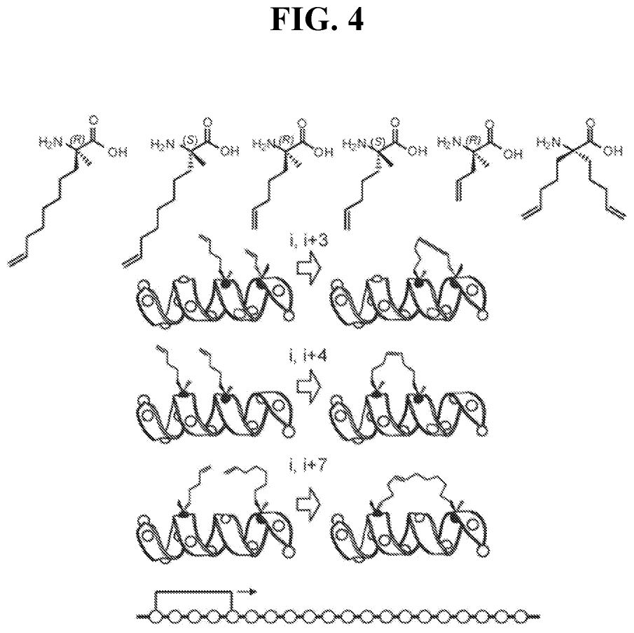

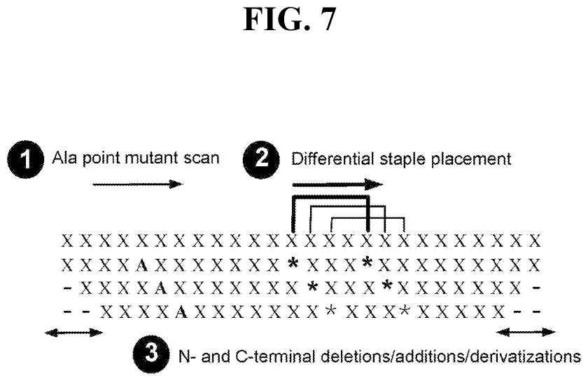

Hydrocarbon-stapled peptides were synthesized, purified, and quantitated using previously reported methods (Bird et al., Methods Enzymol., 446:369-86 (2008); Bird et al., Curr. Protoc. Chem. Biol., 3(3):99-117 (2011). To design peptides that could block the interaction between the SARS-CoV-2 spike protein and the human ACE2 receptor, a series of stapled peptides bearing differentially localized chemical staples and ACE2h1 sequences, including a series of mutants, were designed (FIG. 3, FIG. 8). The differentially localized chemical staples were located within the ACE2 receptor helix 1 peptide that engages the receptor binding domain of SARS-CoV-2 (see FIGS. 1 and 2) by replacing native residues with α, α-disubstituted non-natural olefinic residues (“X”) at select (i, i+4) or (i, i+7) positions and combinations thereof in the form of double staples or stitches, followed by ruthenium-catalyzed olefin metathesis (FIGS. 4-6). Some designs incorporate staples on the non-interacting amphiphilic face of the helix, on the interacting face of the helix, or at positions at the border of the interacting and non-interacting faces of the helix (FIG. 1). Template sequences used were based on the human ACE2 helix 1 peptide, N- and C-terminal truncations thereof, and/or a series of mutations of the native sequence (FIGS. 2, 3, 7, and 8).

Stabilized Alpha-Helix of ACE2h1 (SAH-ACE2h1) constructs were designed by replacing two naturally occurring amino acids with the non-natural S-2-(4′-pentenyl) alanine (S5) amino acids at i, i+4 positions (i.e. flanking 3 amino acids) to generate a staple spanning one α-helical turn, or a combination of (R)-2-(((9H-fluoren-9-yl)methoxy)carbonylamino)-2-methyl-dec-9-enoic acid (R₈) and S5 at i, i+7 positions, respectively, to generate a staple spanning two α-helical turns. Asymmetric syntheses of α, α-disubstituted amino acids were performed as previously described in detail (Schafmeister et al., J. Am. Chem. Soc., 2000; Walensky et al., Science, 2004; Bird et al. Current Protocols in Chemical Biology, 2011, each of which is incorporated by reference in its entirety).

“Staple scanning” was performed to respectively identify residues and binding surfaces critical for interaction, which dictates the design of optimized constructs and negative control mutants (FIG. 4). The N-termini of SAHs were capped with acetyl, a fluorophore (e.g. FITC, rhodamine), or affinity tag (e.g. biotin) depending upon the experimental application.

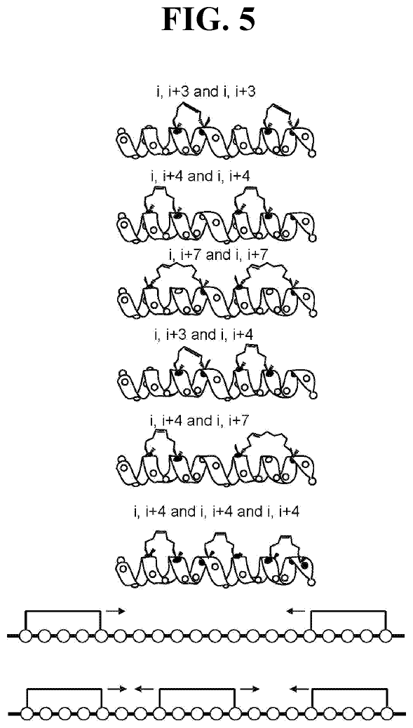

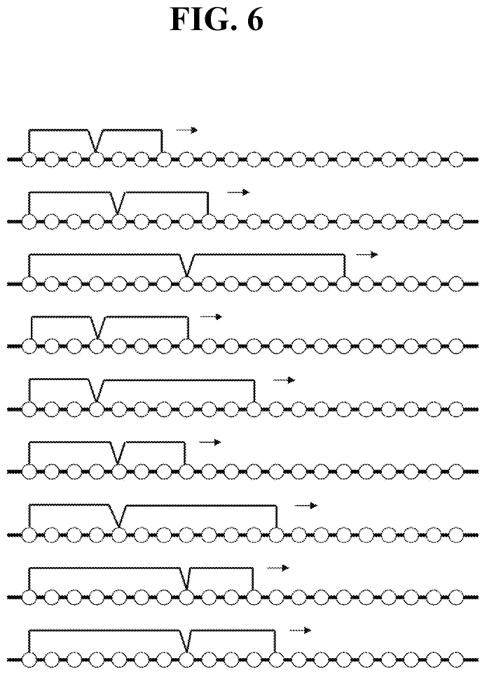

Doubly stapled peptides were generated by installing two-S5-S5, two —R₈—S5, or other combinations of crosslinking non-natural amino acids. Multiply stapled or stitched peptides are generated using similar principles (FIGS. 5 and 6).

Synthesis of the SAH-ACE2h peptides shown in FIGS. 3 and 8 were performed using solid phase Fmoc chemistry and ruthenium-catalyzed olefin metathesis, followed by peptide deprotection and cleavage, purification by reverse phase high performance liquid chromatography/mass spectrometry (LC/MS), and quantification by amino acid analysis (AAA) (Bird et al., Methods Enzymol., 2008).

The peptides shown in Table 3 were synthesized based on the template sequences in Table 1. SEQ ID NOs: 2-12, 145-148, 18-20, 51-60, 134-143, and 172 in Table 1 and FIG. 3 were generated by mutating SEQ ID NO:21 (IEEQAKTFLDKFNHEAEDLFYQSS) with substitutions of naturally-occurring amino acids and/or by substituting amino acids therein with other naturally-occurring amino acids. SEQ ID NOs: 78-111 in FIG. 8 were generated by mutating SEQ ID NO:76 (IEEQAKTFLDKFNHEAEDLFYQS) with substitutions of naturally-occurring amino acids and by substituting amino acids therein with other naturally-occurring amino acids. SEQ ID NOs: 112-126, 134-143, 145-149, and 172 in FIGS. 3 and 8 were generated by mutating SEQ ID NO:21 (IEEQAKTFLDKFNHEAEDLFYQSS) with substitutions of naturally-occurring amino acids and by substituting amino acids therein with other naturally-occurring amino acids. SEQ ID NOs: 127-133 in FIG. 8 were generated by mutating SEQ ID NO:77 (EQAKTFLDKFNHEAEDLFYQ) with substitutions of naturally-occurring amino acids and by substituting amino acids therein with other naturally-occurring amino acids. SEQ ID NOs: 50, 53, 54, and 56 were generated by mutating SEQ ID NO:49 by substituting amino acids therein with other naturally-occurring amino acids (not shown in Table below). SEQ ID NOs: 51, 52, 55, and 57-60 were generated by mutating peptides in SEQ ID NO:49 by substitution with naturally-occurring amino acids and with non-natural all-hydrocarbon cross-link or staple (indicated by “8” or “X” in Table 6).

TABLE 6
Generated Conjugated ACE2 Peptides

| Name | Sequence | SEQ ID NO |
|---|---|---|
| SAH- | IEEQAKTAADKANHEAEQAAYQSAXaa₁Xaa₂, | 56 |
| ACE2h1- | wherein Xaa₁ is L, A, or absent, and Xaa₂ is A or | |
| mut-7 | absent | |
| (P7) | | |
| SAH- | IEEQAKTXADKXNHEAEQAAYQSAXaa₁Xaa₂, | 57 |
| ACE2h1- | wherein Xaa₁ is L, A, or absent, and Xaa₂ is A or | |
| mut-8 | absent | |
| (P8) | | |
| SAH- | IEEQXKTAXDKANHEXEQAXYQSAXaa₁Xaa₂, | 58 |
| ACE2h1- | wherein Xaa₁ is L, A, or absent, and Xaa₂ is A or | |
| mut-9 | absent | |
| (P9) | | |
| SAH- | IEEQXKEAXDKANHEXEQAXYQSAXaa₁Xaa₂, | 59 |
| ACE2h1- | wherein Xaa₁ is L, A, or absent, and Xaa₂ is A or | |
| mut-10 | absent | |
| (P10) | | |
| SAH- | IEEQAKTA8DKANHEXEQAAYQSAXaa₁Xaa₂, | 60 |
| ACE2h1- | wherein Xaa₁ is L, A, or absent, and Xaa₂ is A or | |
| mut-11 | absent | |
| (P11) | | |

Tables 3 and 6 provides a list of synthesized peptide analogs, where X and 8 (bolded e.g., in Table 6) indicate non-natural amino acids used to install the all-hydrocarbon cross-link or staple. In some instances, additional amino acids (e.g., a leucine (L) and/or an alanine (A)) is added to the C-terminus of each peptide. In some instances, non-natural amino acids as shown in Tables 3 and 6 are inserted into one of the peptides of Table 1 to generate the peptide analogs.

In some instances, each peptide in Tables 3 and 6 can include beta alanine at the N-terminus. For example, SEQ ID NO:56 can include BA-IEEQAKTAADKANHEAEQAAYQSAXaa₁Xaa₂, wherein Xaa1 is L, A, or absent, and Xaa₂ is A or absent (SEQ ID NO:171). In some embodiments, a conjugate can be coupled to the N-terminus of any one of the peptides of Tables 3 or 6. In some instances, the conjugate is a detection moiety (e.g., FITC (fluorescein isothiocyanate)). In some instances, the conjugate is a capture moiety (e.g., a biotin moiety).

### Example 2: Assessing Alpha-Helical Stabilization of ACE2h1 Stapled Peptides

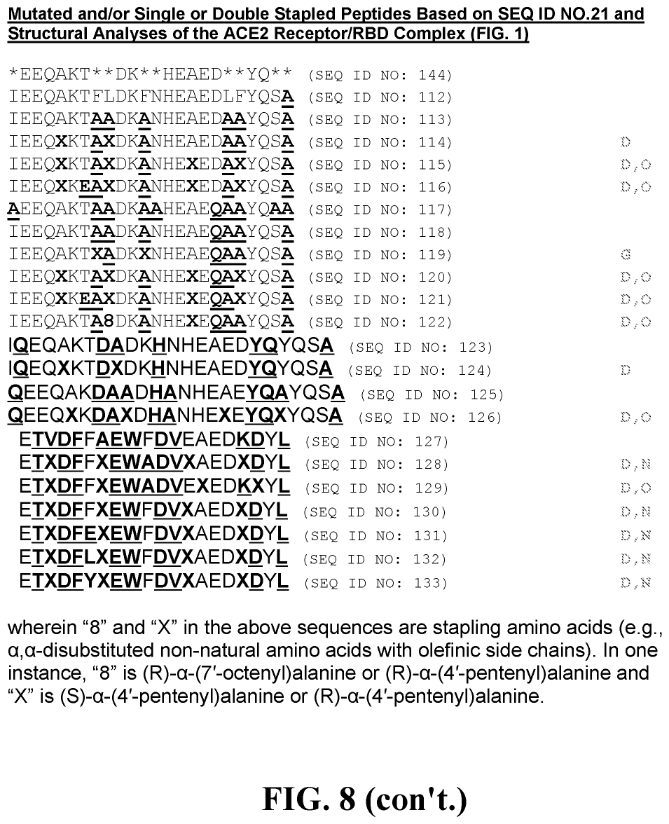

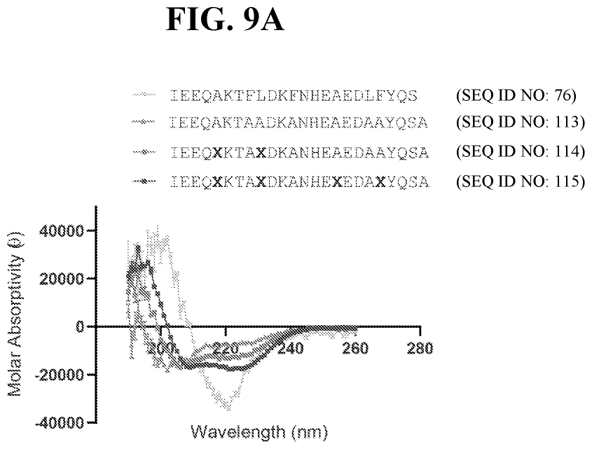

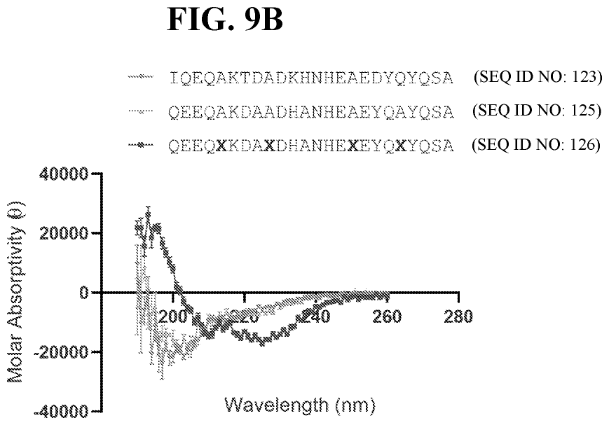

Generally, short peptides do not exhibit significant α-helical structure in solution. This is because the entropic cost of maintaining a conformationally-restricted structure is not overcome by the enthalpic gain from hydrogen bonding of the peptide backbone. To document secondary structure improvements of hydrocarbon-stapled peptides, circular dichroism (CD) spectra was recorded and analyzed on a Model 410 Aviv Biomedical spectrometer. Five scans from 190-260 nm in 0.5 nm increments with 0.5 sec averaging time were collectively averaged to obtain each spectrum using a 1 mm path length cell. The target peptide concentration for CD studies was 25-50 μM in 50 mM potassium phosphate (pH 7.5) or Milli-Q deionized water, and exact concentrations were confirmed by quantitative AAA of two CD sample dilutions. The CD spectra were initially plotted as wavelength versus millidegree. Once the precise peptide concentration was confirmed, the mean residue ellipticity [0], in units of degree·cm²·dmol⁻¹·residue⁻¹, was derived from the equation, [θ]=millidegree/molar concentration/number of amino acid residues. After conversion to mean residue ellipticity, percent α-helicity was calculated using the equation, % helicity=100×[θ]222/max[0]222, where max[θ]222=−40,000×[1−(2.5/number of amino acid residues)]. Stapled constructs that reinforce α-helical structure were advanced to protease-resistance testing, binding analyses, and antiviral activity assays. FIGS. 9A-9B shows how native and mutated ACE2h1 sequences, when synthesized as a peptide and evaluated by circular dichroism, do not retain the natural alpha-helical structure found in the context of the ACE2 receptor. In contrast, inserting staples at specific locations can restore alpha-helical shape. For the ACE2h1 mutant sequence shown in FIG. 9A, a single staple at a specific location (SEQ ID NO:114) can restore alpha-helical shape, with α-helicity improved even further upon double stapling at the indicated locations (SEQ ID NO:115). For a distinct ACE2h1 mutant sequence shown in FIG. 9B, installing double staples in the corresponding positions (SEQ ID NO:126) shown in FIG. 9A, converts the random coil conformation of the unstapled sequences into an α-helix.

### Example 3: Determining Protease Resistance of SARS-CoV-2 HR2 Stapled Peptides

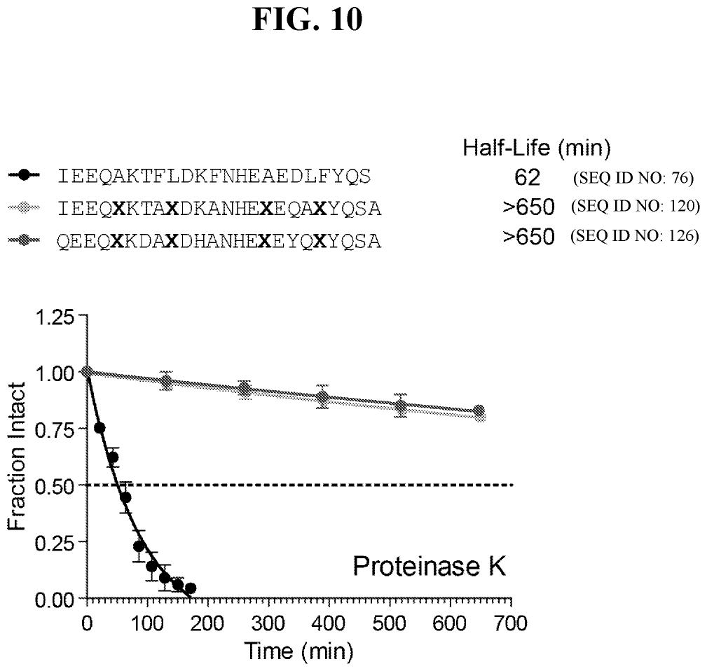

Linear peptides are susceptible to rapid proteolysis in vitro and in vivo, limiting the application of natural peptides for mechanistic analyses and therapeutic use. In contrast, amide bonds engaged in the hydrogen-bonding network of a structured peptide helix are poor enzymatic substrates, as are residues shielded by the hydrocarbon staple itself (Bird et al., PNAS, 2010). To evaluate the relative protease resistance conferred by hydrocarbon stapling, in vitro proteolytic degradation was measured by LC/MS (Agilent 1200) using the following parameters: 20 μL injection, 0.6 mL flow rate, 15 min run time consisting of a gradient of water (0.1% formic acid) to 20-80% acetonitrile (0.075% formic acid) over 10 min, 4 min wash to revert to starting gradient conditions, and 0.5 min post-time. The DAD signal was set to 280 nm with an 8 nm bandwidth and MSD set to scan mode with one channel at (M+2H)/2, +/−1 mass units and the other at (M+3H)/3, +/−1 mass units. Integration of each MSD signal yielded areas under the curve of >10⁸ counts. Reaction samples were composed of 5 μL peptide in DMSO (1 mM stock) and 195 μL of buffer consisting of 50 mM Tris HCl at pH 7.4. Upon injection of the 0 hr time point sample, 2 μL of 100 ng/μL proteinase K (New England Biolabs) was added and the amount of intact peptide quantitated by serial injection over time. An internal control of acetylated tryptophan carboxamide at a concentration of 100 μM is used to normalize each MSD data point. A plot of MSD area versus time yielded an exponential decay curve and half-lives were determined by nonlinear regression analysis using Prism software (GraphPad). FIGS. 13A and 13B show how insertion of double staples or stitches into the core template sequence (aa 1169-1197) conferred striking protease stability compare to the unstapled sequence, depending on the sequence, staple type, and staple location. FIG. 10 shows that insertion of double staples at the indicated positions into two ACE2h1 sequences (SEQ ID NOs: 120 and 126) bearing mutations confers striking in vitro proteinase K resistance to both constructs compared to the unstapled wild-type ACE2h1 sequence.

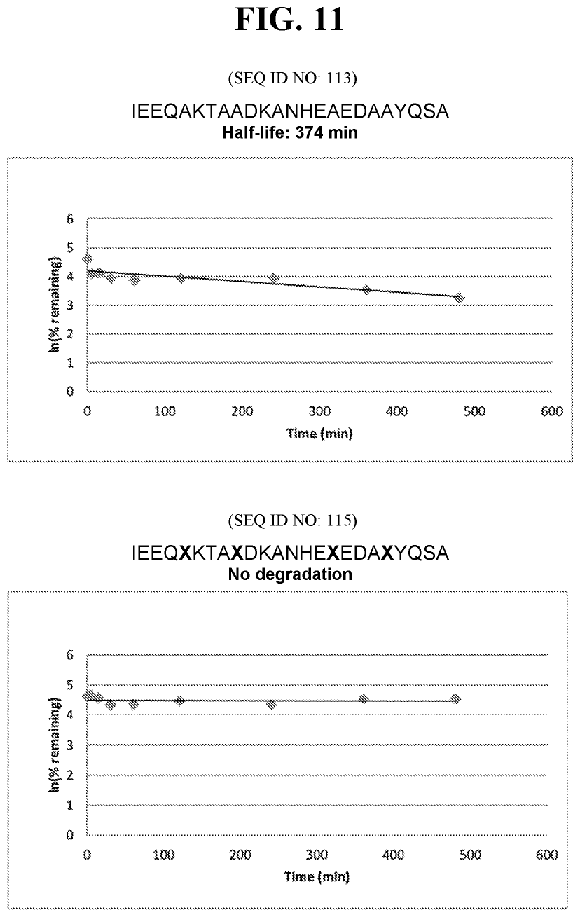

The protease resistance and stability of stapled peptides were also measured by use of a mouse plasma stability assay. Stapled peptide stability was tested in freshly drawn mouse plasma collected in lithium heparin tubes. Triplicate incubations were set up with 500 μl of plasma spiked with 10 μM of the individual peptides. Samples were gently shaken in an orbital shaker at 37° C. and 25 μl aliquots were removed at 0, 5, 15, 30, 60, 240, 360 and 480 min and added to 100 μl of a mixture containing 10% methanol: 10% water: 80% acetonitrile to stop further degradation of the peptides. The samples were allowed to sit on ice for the duration of the assay and then transferred to a MultiScreen Solvinert 0.45 μm low-binding hydrophilic PTFE plate (Millipore). The filtrate was directly analyzed by LC-MS/MS. The peptides were detected as double or triple charged ions using a Sciex 5500 mass spectrometer. The percentage of remaining peptide was determined by the decrease in chromatographic peak area and log transformed to calculate the half-life. FIG. 11 shows the mouse plasma stability of an unstapled ACE2h1 mutant sequence (SEQ ID NO: 113, top chart) and a double-stapled analog (SEQ ID NO: 115, bottom chart). Whereas the unstapled peptide demonstrates a half-life of 374 min, essentially no degradation was observed upon insertion of double staples at the indicated locations.

### Example 4: Visualization of ACE2h Stapled Peptide Binding Activity to SARS-CoV-2 RBD Protein by Bead Assay

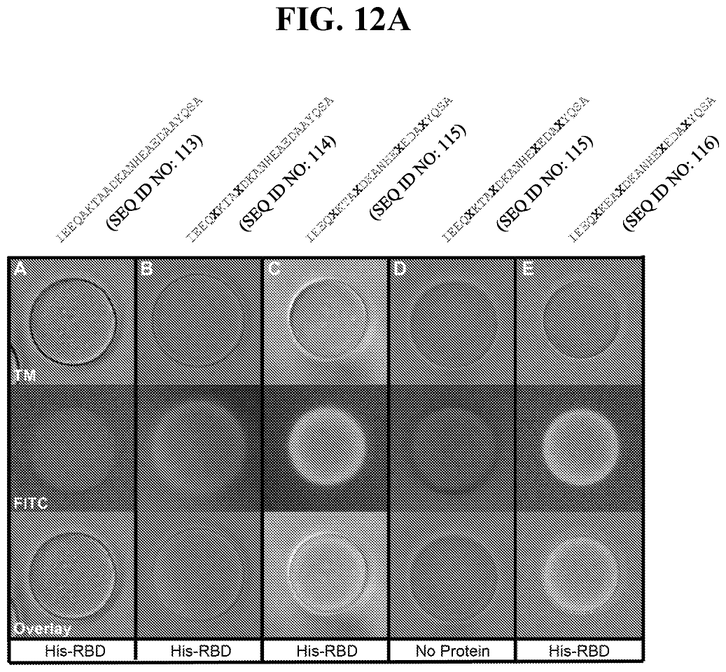

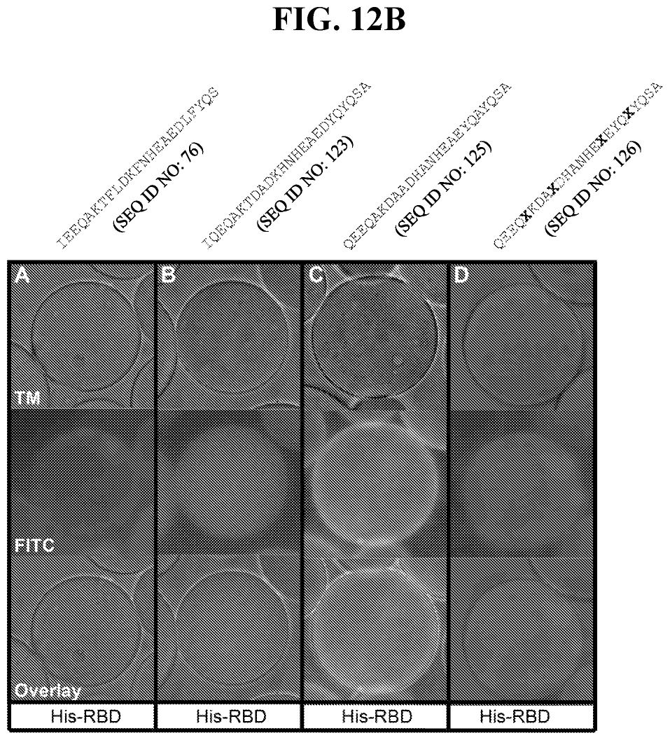

To asses and visualize the binding activity of SAH-ACE2h1 peptides for the SARS-CoV-2 receptor binding domain (RBD), 25 μg of recombinant His-tagged RBD containing amino acids Val 16-Arg 685 of S1 (SEQ ID NO:75) was captured using 100 μl of Ni-NTA agarose beads (Invitrogen™ catalog #R90110) followed by incubation with the FITC-labeled peptides (10 μM) for 30 min. FIG. 12A shows that an unstapled and mutated ACE2h1 peptide has little to no detectable binding to the RBD-coated beads (FIG. 12A, column A, SEQ ID NO: 113), whereas insertion of a single staple at the indicated location (FIG. 12A, column B, SEQ ID NO: 114) and then double staples at the indicated locations (FIG. 12A, columns C and E, SEQ ID NOs: 115 and 116, respectively) leads to progressively enhanced binding activity and RBD detection. Importantly, when no RBD protein is added to the beads, the positive fluorescence signal of an exemplary double stapled ACE2h1 peptide seen with RBD-coated beads (FIG. 12, columns A-C, SEQ ID NOs: 113-115, respectively) is completely lost (FIG. 12A, column D, SEQ ID NO: 115), highlighting the specificity of double stapled peptide binding activity for RBD. FIG. 12B shows that peptide templates bearing a series of mutations can enhance binding to the RBD-coated beads compared to the native sequence (FIG. 12A, columns A-C, SEQ ID NOs: 76, 123, and 125, respectively), including a double stapled analog (FIG. 12A, column D, SEQ ID NO:126).

### Example 5: Utilization of SAH-ACE2h1 Peptides as a Detection Agent for the Diagnosis of SARS-CoV-2

The peptides of the invention can be derivatized with fluorophores, affinity tags, and/or enzymes (e.g. HRP) and incorporated into an assay for the detection of SARS-CoV-2 in human fluid (nasal, oral, respiratory fluid or mucous), as a rapid, capture-and-detect (non-serologic/non-genetic) point-of-care diagnostic test. FIG. 13 shows that the bead-binding results in FIG. 12, namely the capacity of a FITC-labeled and stapled ACE2h1 peptide sequence to bind and detect SARS-CoV-2 RBD protein, can afford simple and rapid virus detection methods. For example, Peptide A (detection tag; e.g. fluorophore) and B (affinity tag; e.g. biotin) are mixed in a solution (FIG. 13A, part A). The patient sample is added to the peptide solution and mixed (FIG. 13A, part B). The virus contains multiple sites to which the differentially-labeled (e.g., comprising a moiety for detection or a moiety for affinity capture) peptides can bind, and therefore, each viral particle can bind to Peptides A and B. The capture beads (e.g. streptavidin beads to capture biotinylated peptide) are added, mixed, and collected by gravity or centrifugation (FIG. 13A, part C). If the result is negative (no virus present) the peptide containing the chromophore/fluorophore remains in solution (FIG. 13A, part D, top). When the result is positive (virus present in sample), the virus is captured by the beads via peptide B, which is collected along with simultaneous virus-bound peptide A, leading to an immediate read-out using a detection instrument (FIG. 13A, part D, bottom). An alternative approach is shown in FIG. 13B and is based on a pregnancy-type strip or ELISA set up in which SAH-ACE2h1 peptide is fixed to a solid support (FIG. 13B, parts A and B; e.g. biotin-peptide saturates a streptavidin coated plate), a patient sample is added to the strip or plate well (FIG. 13B, part C), and then application of a second SAH-ACE2h1 peptide is applied (FIG. 13B, part D) that allows for a colorimetric read-out, such as a second biotinylated SAH-ACE2h1 peptide detected by streptavidin HRP (FIG. 13B, part E) and incubation with chromogenic substrate (FIG. 13B, part F). FIG. 13C demonstrates the successful development of a SAH-ACE2h1-based test strip, based on this concept of an enzyme-linked stapled peptide assay (ELIPSA), which dose-responsively detects a serial dilution of inactivated native SARS-CoV-2 virus (starting titer of 10). Like a pregnancy strip test, this diagnostic ELIPSA method can be used as a simple, rapid, and point-of-care method that does not require complex infrastructure or expertise to diagnose the infection, and thus can also be for home use.

### Example 6: Solution-Phase Binding Analysis of SAH-ACE2h1 Interaction with SARS-CoV-2 RBD and Spike Proteins

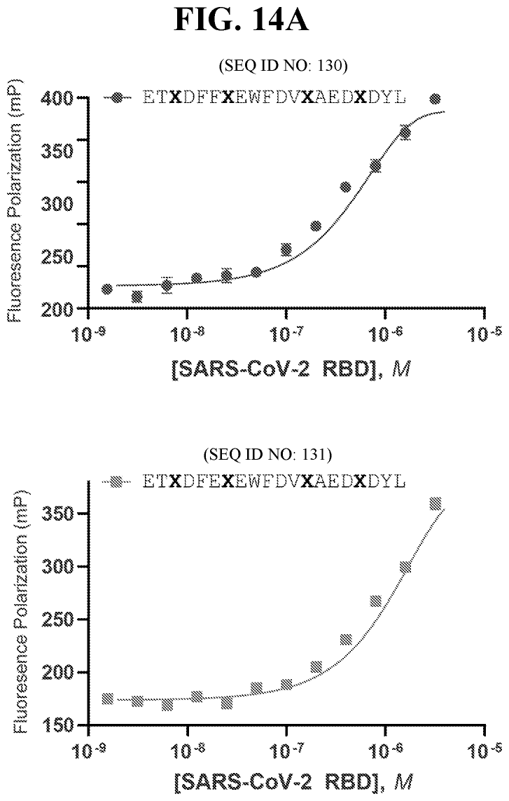

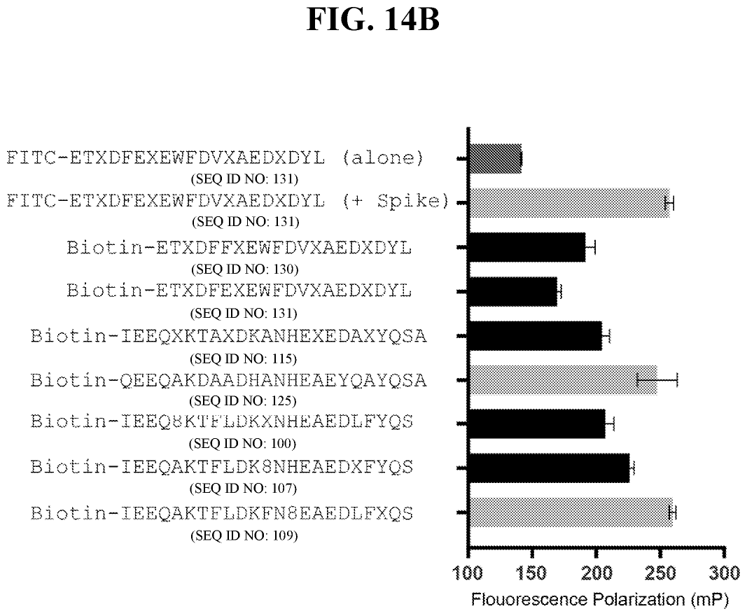

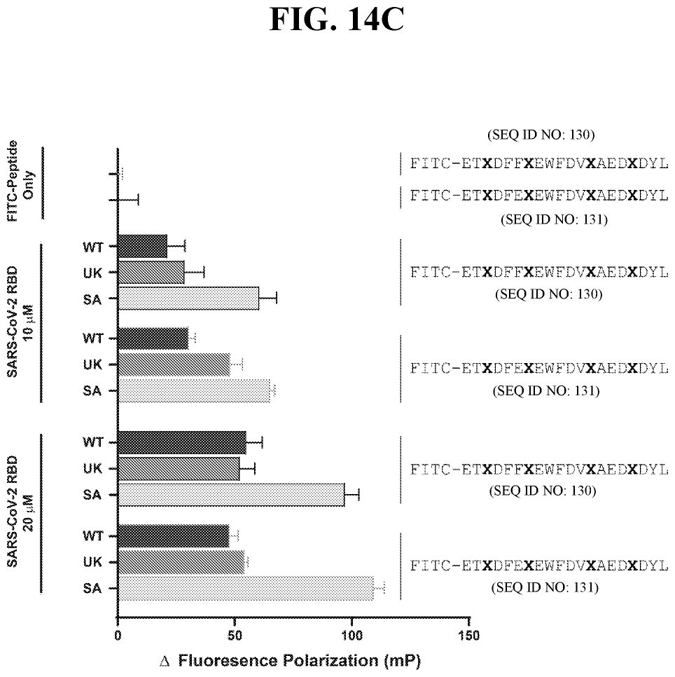

Direct and competitive fluorescence polarization binding assays were used to evaluate and compare the binding activity of SAH-ACE2h1 peptides for SARS-CoV-2 proteins, including recombinant SARS-CoV-2 RBD and Spike proteins. FIGS. 14A-14C shows the binding activities of differentially stapled and mutated ACE2h1 peptides for recombinant SARS-CoV-2 proteins containing the RBD. In FIG. 14A, a direct fluorescence polarization binding assay (FPA) is shown in which FITC-labeled double i, i+4 stapled ACE2h1 peptides bearing a series of mutations demonstrate dose-responsive binding activity when incubated with a serial dilution of purified, recombinant, GST-tagged SARS-CoV-2 RBD protein expressed in E. coli. In FIG. 14B, the direct binding interaction between a FITC-labeled double i, i+4 stapled ACE2h1 peptides bearing a series of mutations and the recombinant SARS-CoV-2 spike protein is used to screen biotinylated (non-fluorescent) and differentially stapled ACE2h1 peptides, with and without mutations, for competitive spike protein binding activity in solution, revealing constructs that were capable (black bar). In FIG. 14C, a direct fluorescence polarization binding assay (FPA) is shown in which two FITC-labeled double i, i+4 stapled ACE2h1 peptides bearing a series of mutations bound to wild-type SARS-CoV-2 RBD protein expressed in mammalian HEK293 cells and retained at least equivalent, or exhibit more, binding activity to SARS-CoV-2 RBD proteins bearing clinical variants such as the UK (N501Y) and South African (K417N, E484K and N501Y), with the latter variant protein in particular showing enhanced binding activity to both SAH-ACE2h1 peptides.

### Example 7: Solid-Phase Binding Analysis of SAH-ACE2h1 Interaction with SARS-CoV-2 RBD Protein

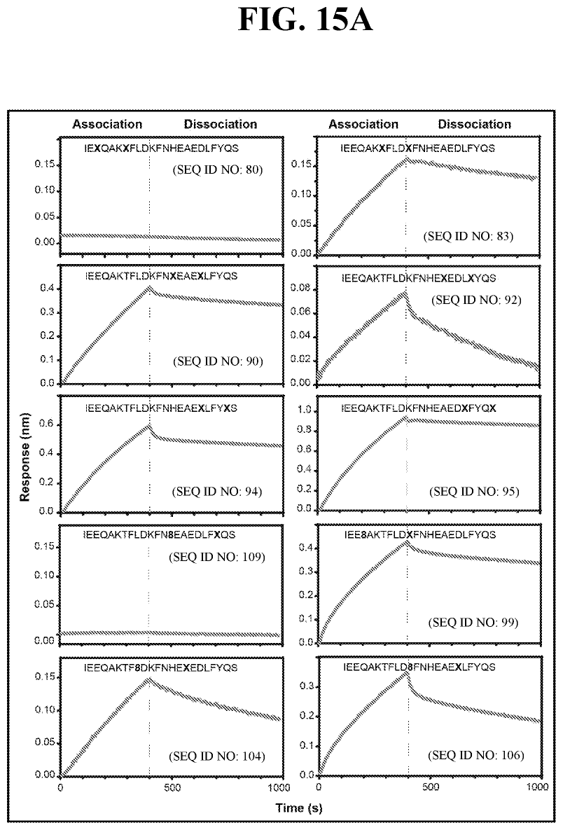

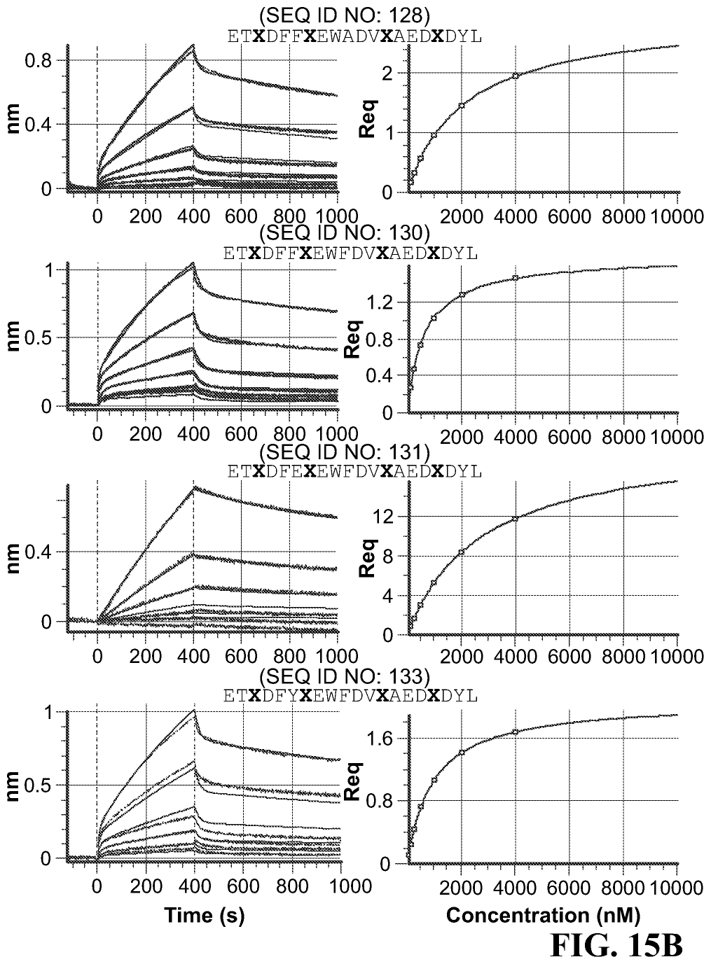

Applying SAH-ACE2h1 peptide to a solid support, such as via biotin-peptide capture by a streptavidin coated tip or chip, can be used to evaluate and compare the binding activity of SAH-ACE2h1 peptides for SARS-CoV-2 RBD protein using methods such as biolayer interferometry (BLI) or surface plasmon resonance. FIGS. 15A-15B show a binding analysis in which biotinylated stapled ACE2h1 peptides were applied to a streptavidin-coated tip (solid support) and tested for recombinant SARS-CoV-2 RBD binding activity by BLL. In FIG. 15A, single i, i+4 or single i, i+7 stapled ACE2h1 peptides were tested for SARS-CoV-2 RBD binding activity at a screening dose, revealing compounds that do or do not bind to RBD based on peptide sequence, staple type, and/or staple position. In FIG. 15B, double i, i+4 stapled ACE2h1 peptides bearing a series of mutations showed dose-responsive RBD binding activity.

### Example 8: Antiviral Activity of SAH-ACE2h1 Peptides as Assessed by Pseudovirus Infection Assay

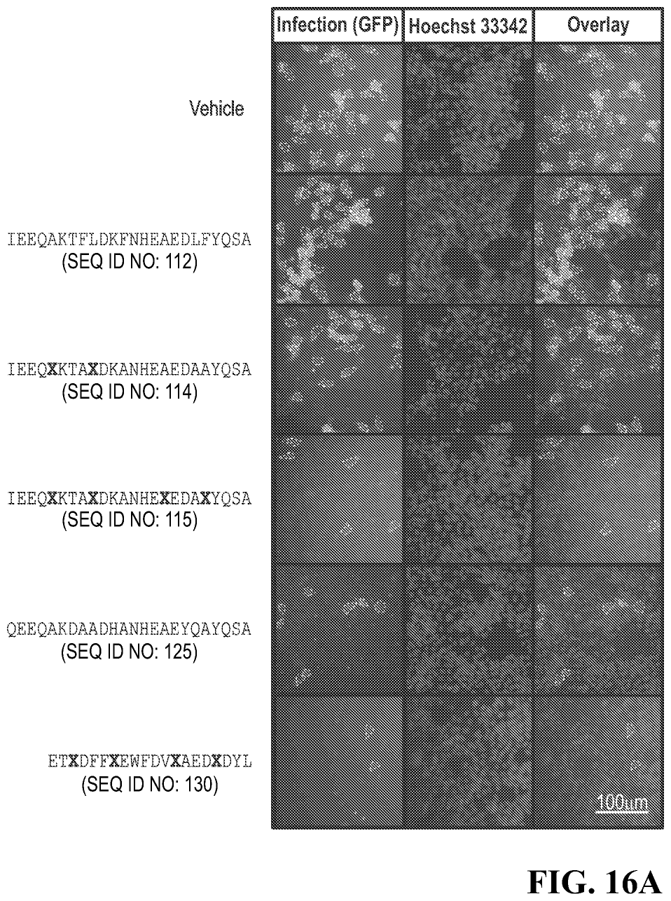

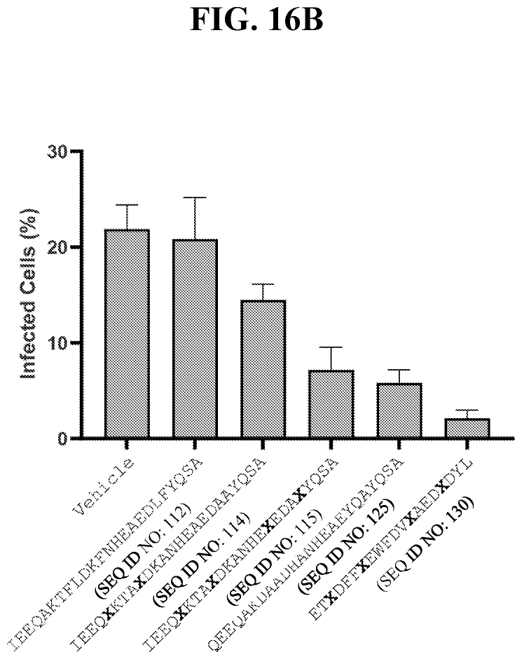

Antiviral activity of ACE2 α1 helix stapled peptides were assessed using pseudotyped virus. The 293T-hsACE2 stable cell line (Cat #C-HA101) and the pseudotyped SARS-CoV-2 (Wuhan-Hu-1 strain) particles with GFP (Cat #RVP-701G, Lot #CG-113A) reporters were used (Integral Molecular). The neutralization assay was carried out according to the manufacturers' protocols. In brief, 5 μL of a single dose of peptide (5 μM final dose) was incubated with 5 μL pseudotyped SARS-CoV-2-GFP for 1 hr at 37° C. in a 384 well black clear bottom plate followed by addition of 30 μL of 1,000 293T-hsACE2 cells in 10% FBS DMEM, phenol red free media and placed in a humidified incubator for 48 or 72 hrs. Hoechst 33342 and DRAQ7 dyes were added and the plate imaged on a Molecular Devices ImageXpress Micro Confocal Laser at 10× magnification. GFP (+) cells were counted and plotted using Prism software (Graphpad). FIGS. 16A-16B show a SARS-CoV-2 pseudovirus assay in which GFP-coding virus was used to infect 293T cells that were treated with ACE2h1 peptides, followed by fluorescence microscopy imaging to detect and compare the level of virus between vehicle and peptide treatments to assay for inhibition of viral infection (FIG. 16A), which was quantitated by image analysis (FIG. 16B). Insertion of a single staple conferred antiviral activity compared to the unstapled template peptide that showed no activity, with double stapling enhancing antiviral activity further. Installing a series of mutations into the template sequence or double stapling an N- and C-terminally truncated construct bearing a series of distinct mutations also conferred marked antiviral activity in the pseudovirus assay.

### Example 9: Antiviral Activity of SAH-ACE2h1 Peptides as Assessed by a Native SARS-CoV-2 Infectivity Assay

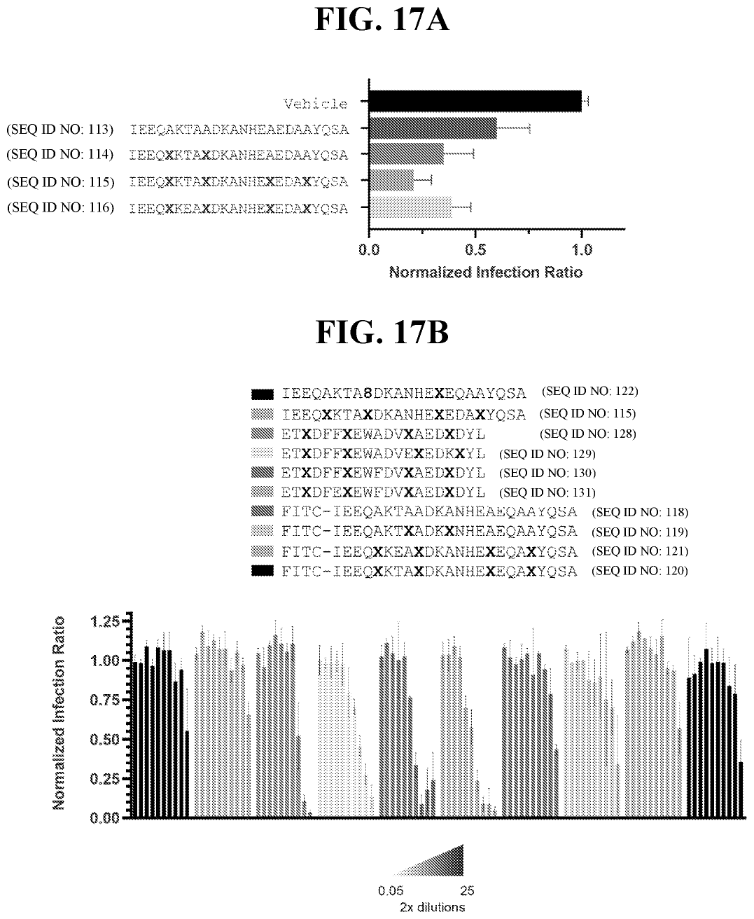

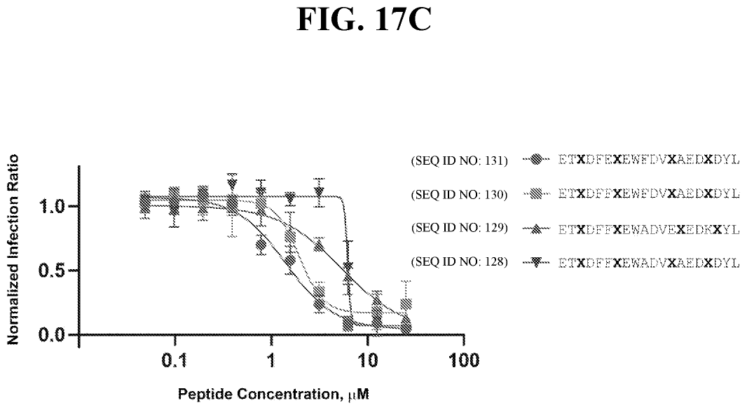

Vero E6 cells plated in 384-well format were treated for 1 hour with either a screening dose of stapled peptide (e.g. 25 μM) or a serial dilution of stapled peptide (e.g. 25 μM starting dose), performed in triplicate, followed by 4 hour challenge with SARS-CoV-2 to achieve control infection of 10-20% cells (the pre-determined optimal infectivity to assess the dynamic range of test compounds in the assay). Infected cells were then washed, fixed with 4% paraformaldehyde, rewashed in PBS, immune-stained with anti-SARS-CoV-2 nucleocapsid monoclonal antibody followed by anti-Ig secondary antibody (Alexa Fluor 488; Life Technologies), and cell bodies counterstained with HCS CellMask blue. Cells were imaged across the z-plane on a Nikon Ti Eclipse automated microscope, analyzed by CellProfiler software, and infection efficiency calculated by dividing infected by total cells. Control cytotoxicity assays were performed using Cell-Titer Glo (Promega) and LDH release (Roche) assays. FIG. 17A shows that initial screening for inhibitory activity at a 25 μM test dose demonstrated that single and double stapled constructs of a template ACE2h1 sequence exhibited improved antiviral activity. Positive hits were advanced to serial dilution testing. FIG. 17B shows the differential antiviral activity of a series of SAH-ACE2h1 peptides bearing single i, i+4 or i, i+7 staples, or double i, i+4 staples, in the context of various ACE2h1 template sequences bearing a serious of distinct mutations. FIG. 17C shows additional biological replicates of i, i+4 double stapled peptides of ACE2h1 template sequences bearing a series of mutations and exhibiting dose-responsive antiviral activity.

### Example 10: Identification of Lead SAH-ACE2h1 Peptides by Correlation of Biochemical and Antiviral Activities

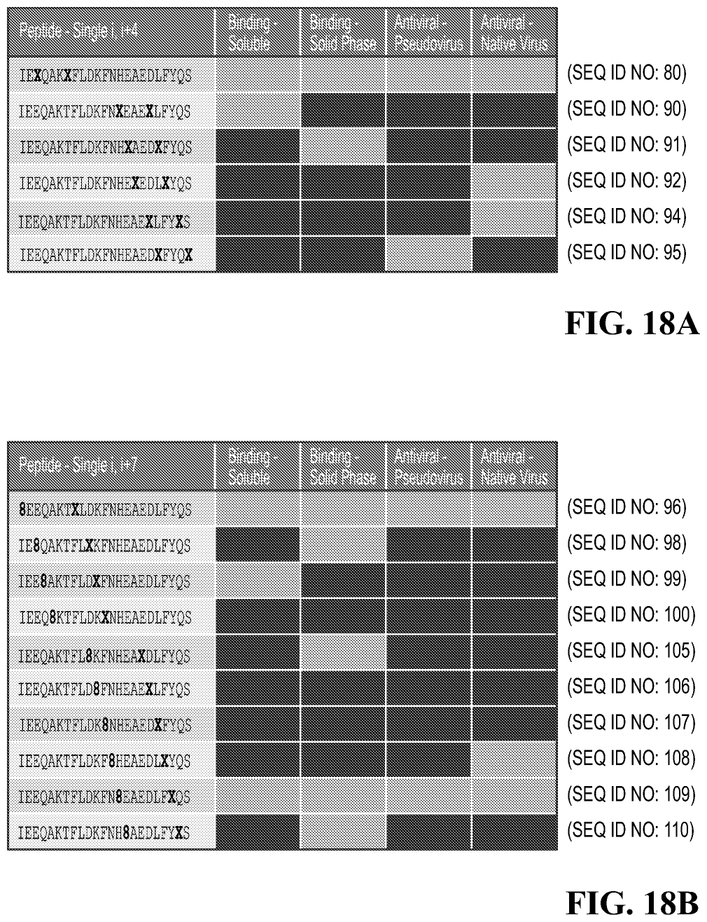

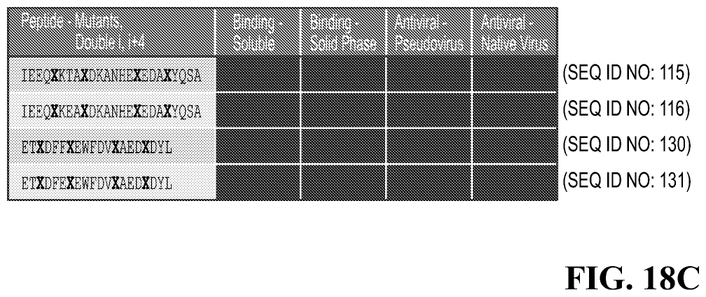

Synthesis and multidisciplinary testing of libraries of stapled peptides based on diverse sequence templates, which can incorporate individual or a series of mutations, can enable the identification of lead constructs that demonstrate correlations between optimized biophysical properties, target binding affinities, and functional antiviral activity. FIGS. 18A-18C show how lead SAH-ACE2h1 peptides were identified based on consistency of activity across a diversity of assays, including binding assays with peptides in solution or on solid support and antiviral assays using SARS-CoV-2 pseudovirus or native virus. FIG. 18A shows an exemplary single i, i+4 stapled peptide of the native sequence with no biological activity across 4 assays and 5 single i, i+4 stapled peptides of the native sequence that demonstrated biological activity in 3 of 4 functional assays. FIG. 18B shows two exemplary i, i+7 single stapled peptides of the native sequence with no biological activity across 4 assays and 8 single i, i+7 single stapled peptides of the native sequence that demonstrated biological activity in at least 3 of 4 functional assays, with 3 compositions demonstrating efficacy across all RBD binding and antiviral assays. FIG. 18C shows how incorporation of favorable staple types and locations based on staple scanning into templates iterated by amino acid mutagenesis led to identification of a series of lead constructs with consistent biological activity across 4 independent assays, spanning soluble binding, solid phase binding, pseudovirus infectivity, and native virus infectivity assays.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims

1. A polypeptide comprising an amino acid sequence that is at least 30% and less than 81% identical to, or that has at least 4 and up to 14 amino acid additions or substitutions in, EQAKTFLDKFNHEAEDLFYQ (SEQ ID NO:77), wherein the polypeptide has one or more of the following properties:
(i) binds the peptide of SEQ ID NO: 64 or SEQ ID NO: 65;
(ii) inhibits interaction between human angiotensin converting enzyme 2 (ACE2) protein and an S1 protein subunit of SARS-CoV-2;
(iii) inhibits interaction between a carboxypeptidase domain of human ACE2 protein and the S1 protein subunit of SARS-CoV-2;
(iv) inhibits interaction between the carboxypeptidase domain of human ACE2 protein and a receptor binding domain (RBD) of the S1 protein subunit of SARS-CoV-2;
(v) competes for human ACE2-SARS-CoV-2 S1 protein binding;
(vi) binds S1 protein of a SARS-CoV-2 variant and/or RBD of the SARS-CoV-2 variant; and
(vii) inhibits a SARS virus infection, optionally, wherein the SARS infection is SARS-CoV-2 infection.

2.-3. (canceled)

4. The polypeptide of claim 1, comprising the amino acid sequence:
(i)
(SEQ ID NO: 125)
QEEQAKDAADHANHEAEYQAYQSA,
(ii)
(SEQ ID NO: 113)
IEEQAKTAADKANHEAEDAAYQSA,
(iii)
(SEQ ID NO: 118)
IEEQAKTAADKANHEAEQAAYQSA,
iv)
(SEQ ID NO: 117)
AEEQAKTAADKAAHEAEQAAYQAA,
(v)
(SEQ ID NO: 123)
IQEQAKTDADKHNHEAEDYQYQSA,
or
(vi)
(SEQ ID NO: 127)
ETVDFFAEWFDVEAEDKDYL[[;]].

5. The polypeptide of claim 1, comprising the amino acid sequence of any one of the sequences set forth in SEQ ID NOs.: 90-95, 98-100, 105-108, and 110.

6. (canceled)

7. The polypeptide of claim 1, wherein the polypeptide is hydrocarbon stapled or stitched.

8. The polypeptide of claim 1, which is not stapled or stitched.

9. The polypeptide of claim 1, wherein the polypeptide comprises an amino acid sequence set forth in any one of SEQ ID NOs: 10-12, 17-20, and 113-133.

10. The polypeptide of claim 7, comprising the amino acid sequence set forth in:
(i)
(SEQ ID NO: 115)
IEEQXKTAXDKANHEXEDAXYQSA,
(ii)
(SEQ ID NO: 116)
IEEQXKEAXDKANHEXEDAXYQSA,
(iii)
(SEQ ID NO: 120)
IEEQXKTAXDKANHEXEQAXYQSA,
(iv)
(SEQ ID NO: 121)
IEEQXKEAXDKANHEXEQAXYQSA,
(v)
(SEQ ID NO: 126)
QEEQXKDAXDHANHEXEYQXYQSA,
(vi)
(SEQ ID NO: 132)
ETXDFLXEWFDVXAEDXDYL,
(vii)
(SEQ ID NO: 133)
ETXDFYXEWFDVXAEDXDYL,
(viii)
(SEQ ID NO: 130)
ETXDFFXEWFDVXAEDXDYL,
[[or]]
(ix)
(SEQ ID NO: 131)
ETXDFEXEWFDVXAEDXDYL,
(x)
(SEQ ID NO: 128)
ETXDFFXEWADVXAEDXDYL,
(xii)
(SEQ ID NO: 129)
ETXDFFXEWADVEXEDKXYL,
(xiii)
(SEQ ID NO: 100)
IEEQXKTFLDKXNHEAEDLFYQS,
(xiv)
(SEQ ID NO: 105)
IEEQAKTFLXKFNHEAXDLFYQS,
(XV)
(SEQ ID NO: 106)
IEEQAKTFLDXFNHEAEXLFYQS,
or
(xvi)
(SEQ ID NO: 107)
IEEQAKTFLDKXNHEAEDXFYQS,
wherein each X in the sequences above is an α, α-disubstituted non-natural amino acid with olefinic side chains.

11. The polypeptide of claim 7, further comprising α, α-disubstituted non-natural amino acids with olefinic side chains that are internally cross-linked,
wherein the α, α-disubstituted non-natural amino acids are inserted at positions 3 and 7 and/or positions 14 and 15 of SEQ ID NO:77 (wherein the position numbering is from the N-terminal E (position 1) to the C-terminal Q (position 20), or
wherein the α, α-disubstituted non-natural amino acids are inserted at positions 3 and 7 and/or positions 13 and 17 of SEQ ID NO:77 (wherein the position numbering is from the N-terminal E (position 1) to the C-terminal Q (position 20).

12. (canceled)

13. The polypeptide of claim 7, further comprising α, α-disubstituted non-natural amino acids with olefinic side chains that can be internally cross-linked, wherein the α, α-disubstituted non-natural amino acids are inserted at positions SEQ ID NO:77 (wherein the position numbering is from the N-terminal E (position 1) to the C-terminal Q (position 20):
(i) positions 5 and 12,
(ii) positions 11 and 18,
(iii) positions 12 and 19,
(iv) positions 14 and 18,
(v) positions 15 and 19, or
(vi) positions 16 and 20.

14. (canceled)

15. The polypeptide of claim 1, wherein the polypeptide is not substituted at one or more of positions 1, 6, 10, 14, 15, 16 and 19 of SEQ ID NO:77 (wherein the position numbering is from the N-terminal E (position 1) to the C-terminal Q (position 20).

16. The polypeptide of claim 1, which is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

17. (canceled)

18. A polypeptide comprising an amino acid sequence set forth below:
(i)
(SEQ ID NO: 2)
X₁EEQX₂KTFLDKFNHEAEDLFYQSSXaa₁Xaa₂,
(ii)
(SEQ ID NO: 3)
IEEQX₁KTFX₂DKFNHEAEDLFYQSSXaa₁Xaa₂,
(iii)
(SEQ ID NO: 4)
IEEQAKTX₁LDKX₂NHEAEDLFYQSSXaa₁Xaa₂,
(iv)
(SEQ ID NO: 5)
IEEQAKTFLDKX₁NHEX₂EDLFYQSSXaa₁Xaa₂,
(v)
(SEQ ID NO: 6)
IEEQAKTFLDKFNHEX₁EDLX₂YQSSXaa₁Xaa₂,
(vi)
(SEQ ID NO: 7)
IEEQAKTF8DKFNHEXEDLFYQSSXaa₁Xaa₂,
(vii)
(SEQ ID NO: 8)
IEEQ8KTFLDKXNHEAEDLFYQSSXaa₁Xaa₂,
(viii)
(SEQ ID NO: 9)
IEEQAKTFLDK8NHEAEDXFYQSSXaa₁Xaa₂,
(ix)
(SEQ ID NO: 10)
X₁EEQX₂KTFLDKFNHEX₃EDLX₄YQSSXaa₁Xaa₂,
(x)
(SEQ ID NO: 11)
X₁EEQX₂KTFLDKX₃NHEX₄EDLFYQSSXaa₁Xaa₂,
(xi)
(SEQ ID NO: 12)
IEEQX₁KTFX₂DKFNHEX₃EDLX₄YQSSXaa₁Xaa₂,
(xii) Xaa₁₁EEQXaa₁₂KXaa₁₃Xaa₁₄Xaa₁₅DKXaa₁₆Xaa₁₇HEXaa₁₈EDXaa₁₉Xaa₂₀ YXa₂₁Xaa₂₂Xaa₂₃Xaa₂₄Xaa₂₅, wherein
Xaa₁₁=I, A, or a stapling amino acid
Xaa₁₂=A or a stapling amino acid
Xaa₁₃=T, E, or F
Xaa₁₄=F, A, or a stapling amino acid
Xaa₁₅=L, A, or a stapling amino acid
Xaa₁₆=F, A, or a stapling amino acid
Xaa₁₇=N or A
Xaa₁₈=A or a stapling amino acid
Xaa₁₉=L, A, or a stapling amino acid
Xaa₂₀=F, A, or a stapling amino acid
Xaa₂₁=Q or Y
Xaa₂₂=S or A
Xaa₂₃=S or A
Xaa₂₄=L, A, or absent
Xaa₂₅=A or absent (SEQ ID NO:17),
(xiii) Xaa₁₁Xaa₁₂EQXaa₁₃Xaa₁₄TXaa₁₅Xaa₁₆DKXaa₁₇Xaa₁₈HEXaa₁₉Xaa₂₀Xaa₂₁ Xaa₂₂Xaa₂₃YQXaa₂₄Xaa₂₅LXaa₂₆, wherein
Xaa₁₁=I, A, or a stapling amino acid
Xaa₁₂=E or A
Xaa₁₃=A or a stapling amino acid
Xaa₁₄=K, R, or A
Xaa₁₅=F, A, or a stapling amino acid
Xaa₁₆=L, A, or a stapling amino acid
Xaa₁₇=F, A, or a stapling amino acid
Xaa₁₈=N or A
Xaa₁₉=A or a stapling amino acid
Xaa₂₀=E, D, or A
Xaa₂₁=D, E, or A
Xaa₂₂=L, A, or a stapling amino acid
Xaa₂₃=F, A, or a stapling amino acid
Xaa₂₄=S or A
Xaa₂₅=S or A
Xaa₂₆=A or absent (SEQ ID NO: 18),
(xiv) IEEQAK₆TFLD₁₀K₁₁FNHEAED₁₈LFYQSSLA, wherein K₆ and D₁₀ are linked by a lactam bridge; or wherein K₁₁ and D₁₈ are linked by a lactam bridge (SEQ ID NO: 19),
(xv) Xaa₁₁EEQXaa₁₂K₆Xaa₁₃Xaa₁₄Xaa₁₅D₁₀K₁₁Xaa₁₆Xaa₁₇HEXaa₁₈ED₁₈ Xaa₁₉Xaa₂₀YXaa₂₁Xaa₂₂Xaa₂₃Xaa₂₄Xaa₂₅, wherein
Xaa₁₁=I or A,
Xaa₁₂=A
Xaa₁₃=T, E, or F
Xaa₁₄=F or A
Xaa₁₅=L or A
Xaa₁₆=F or A
Xaa₁₇=N or A
Xaa₁₈=A
Xaa₁₉=L or A
Xaa₂₀=F or A
Xaa₂₁=Q or Y
Xaa₂₂=S or A
Xaa₂₃=S or A
Xaa₂₄=L or A
Xaa₂₅=A or absent,
wherein K₆ and D₁₀ are linked by a lactam bridge; or wherein K₁₁ and Dis are linked by a lactam bridge (SEQ ID NO: 20),
(xvi)
(SEQ ID NO: 78)
XEEQXKTFLDKFNHEAEDLFYQS,
(xvii)
(SEQ ID NO: 79)
IXEQAXTFLDKFNHEAEDLFYQS,
(xviii)
(SEQ ID NO: 80)
IEXQAKXFLDKFNHEAEDLFYQS,
(xix)
(SEQ ID NO: 81)
IEEQXKTFXDKFNHEAEDLFYQS,
(xx)
(SEQ ID NO: 82)
IEEQAXTFLXKFNHEAEDLFYQS,
(xxi)
(SEQ ID NO: 83)
IEEQAKXFLDXFNHEAEDLFYQS,
(xxii)
(SEQ ID NO: 84)
IEEQAKTXLDKXNHEAEDLFYQS,
(xxiii)
(SEQ ID NO: 85)
IEEQAKTFXDKFXHEAEDLFYQS,
(xxiv)
(SEQ ID NO: 86)
IEEQAKTFLXKFNXEAEDLFYQS,
(xxv)
(SEQ ID NO: 87)
IEEQAKTFLDXFNHXAEDLFYQS,
(xxvi)
(SEQ ID NO: 88)
IEEQAKTFLDKXNHEXEDLFYQS,
(xxvii)
(SEQ ID NO: 89)
IEEQAKTFLDKFXHEAXDLFYQS,
(xxviii)
(SEQ ID NO: 90)
IEEQAKTFLDKFNXEAEXLFYQS,
(xxix)
(SEQ ID NO: 91)
IEEQAKTFLDKFNHXAEDXFYQS,
(xxx)
(SEQ ID NO: 92)
IEEQAKTFLDKFNHEXEDLXYQS,
(xxxi)
(SEQ ID NO: 93)
IEEQAKTFLDKFNHEAXDLFXQS,
(xxxii)
(SEQ ID NO: 94)
IEEQAKTFLDKFNHEAEXLFYXS,
(xxxiii)
(SEQ ID NO: 95)
IEEQAKTFLDKFNHEAEDXFYQX,
(xxxiv)
(SEQ ID NO: 96)
8EEQAKTXLDKFNHEAEDLFYQS,
(xxxv)
(SEQ ID NO: 97)
I8EQAKTFXDKFNHEAEDLFYQS,
(xxxvi)
(SEQ ID NO: 98)
IE8QAKTFLXKFNHEAEDLFYQS,
(xxxvii)
(SEQ ID NO: 99)
IEE8AKTFLDXFNHEAEDLFYQS,
(xxxviii)
(SEQ ID NO: 100)
IEEQ8KTFLDKXNHEAEDLFYQS,
(xxxix)
(SEQ ID NO: 101)
IEEQA8TFLDKFXHEAEDLFYQS,
(xl)
(SEQ ID NO: 102)
IEEQAK8FLDKFNXEAEDLFYQS,
(xli)
(SEQ ID NO: 103)
IEEQAKT8LDKFNHXAEDLFYQS,
(xlii)
(SEQ ID NO: 104)
IEEQAKTF8DKFNHEXEDLFYQS,
(xliii)
(SEQ ID NO: 105)
IEEQAKTFL8KFNHEAXDLFYQS,
(xliv)
(SEQ ID NO: 106)
IEEQAKTFLD8FNHEAEXLFYQS,
(xlv)
(SEQ ID NO: 107)
IEEQAKTFLDK8NHEAEDXFYQS,
(xlvi)
(SEQ ID NO: 108)
IEEQAKTFLDKF8HEAEDLXYQS,
(xlvii)
(SEQ ID NO: 109)
IEEQAKTFLDKFN8EAEDLFXQS,
(xlviii)
(SEQ ID NO: 110)
IEEQAKTFLDKFNH8AEDLFYXS,
or
(xlix)
(SEQ ID NO: 111)
IEEQAKTFLDKFNHE8EDLFYQX,
wherein X₁, X₂, X₃, X₄, X, and 8 are non-natural amino acids with olefinic side chains;
(i) binds the peptide of SEQ ID NO: 64 or SEQ ID NO: 65;
(ii) inhibits interaction between human ACE2 protein and an S1 protein subunit of SARS-CoV-2;
(iii) inhibits interaction between the carboxypeptidase domain of human ACE2 protein and the S1 protein subunit of SARS-CoV-2;
(iv) inhibits interaction between the carboxypeptidase domain of human ACE2 protein and an RBD of the S1 protein subunit of SARS-CoV-2;
(v) competes for human ACE2-SARS-CoV-2 S1 protein binding;
(vi) binds an S1 protein of a SARS-CoV-2 variant and/or the RBD of the S1 protein subunit of the SARS-CoV-2 variant; and
(vii) inhibits SARS virus infection.

19. A pharmaceutical composition comprising the polypeptide of claim 1 and a pharmaceutically acceptable carrier.

20.-21. (canceled)

22. A nanoparticle composition comprising the polypeptide of claim 1.

23. A diagnostic reagent for detecting coronavirus whose receptor-binding domain binds to ACE2, the diagnostic reagent comprising a surface comprising the polypeptide of claim 1.

24. (canceled)

25. A method for treating or preventing a viral infection caused by (i) a coronavirus infection, or (ii) a virus that infects cells by binding to ACE2 or treating post-acute sequelae of SARS-CoV-2 infection in a subject in need thereof, the method comprising administering to a subject in need thereof a therapeutically effective amount of the polypeptide of claim 1.

26.-28. (canceled)

29. A method of treating a subject in need thereof, the method comprising administering to the subject a therapeutically effective amount of the polypeptide of claim 1,
wherein the subject has been, or is at risk of being, infected with a variant of SARS-CoV-2, or
wherein the subject has post-acute sequelae of SARS-CoV-2 infection.

30. A method of preventing or inhibiting interaction between the receptor binding domain of a virus and ACE2 in a subject in need thereof, the method comprising administering to the subject a therapeutically effective amount of the polypeptide of claim 1.

31.-33. (canceled)

34. A method of making an internally cross-linked polypeptide, the method comprising:
(a) providing the polypeptide of claim 1; and
(b) cross-linking the polypeptide by a ruthenium catalyzed metathesis reaction thereby generating an internally cross-linked polypeptide, and
(c) formulating the internally cross-linked polypeptide as a sterile pharmaceutical composition.

35. A method of detecting the presence of a virus whose receptor binding domain binds to ACE2, the method comprising:
(I) (a) providing a biological sample of a subject;
(b) mixing the biological sample with a plurality of peptides comprising the polypeptide of claim 1 to create a mixture, wherein:
at least one stabilized peptide in the plurality comprises a detection moiety; and
at least one stabilized peptide in the plurality comprises a capture moiety;
(c) providing a diagnostic reagent for detecting the presence of a virus whose receptor-binding domain binds to ACE2;
(d) contacting the diagnostic reagent with the mixture; and
(e) detecting the presence or absence of the virus; or
(II) (a) providing a detection agent, wherein the detection agent is a first polypeptide of claim 1, wherein the first polypeptide binds to the receptor binding domain of the virus, and wherein the first polypeptide is linked to a detection label;
(b) providing a capture agent wherein the capture agent is a second polypeptide of claim 1, wherein the second polypeptide binds to the receptor binding domain of the virus, and wherein the second polypeptide is linked to an affinity label;
(c) mixing a biological sample from a subject with the detection agent and the capture agent to form a mixture:
(d) contacting the mixture with a solid support that binds the capture agent; and
(e) detecting presence or absence of the virus.

36. A compound comprising a stabilized peptide comprising a sequence having the formula: 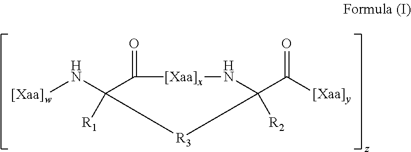
or a pharmaceutically acceptable salt thereof, wherein:
(a) each R₁ and R₂ is independently H, alkyl, alkenyl, alkynyl, arylalkyl, cycloalkylalkyl, heteroarylalkyl, or heterocyclylalkyl, any of which is substituted or unsubstituted;
(b) each R₃ is independently alkylene, alkenylene, or alkynylene, any of which is substituted or unsubstituted;
(c) each x is independently 2, 3, or 6;
(d) each w and y is independently 1, 2, 3, 4, 5, 6, 7, 8, 9, 10, 11, 12, 13, 14, 15, 16, 17, 18, 19, 20, 21, 22, 23, or 24;
(e) z is 1, 2, or 3; and
(f) each Xaa is independently an amino acid,
wherein the sequence comprises the polypeptide of claim 1, with:
(i) 2 to 5 amino acids that are substituted with α, α-disubstituted non-natural amino acids with olefinic side chains that form an internal cross-link,
(ii) 0, 1, 2, 3, 4, 5, 6, 7, 8, 9, 10, 11, 12, 13, or 14 additional amino acid substitutions;
(iii) 0, 1, 2, 3, 4, or 5 deletions at the N and/or C-terminus of the sequence; and/or
(iv) 0, 1, 2, 3, 4, or 5 amino acid additions at the N and/or C-terminus of the sequence, and wherein the stabilized peptide has one or more of the following properties:
(i) binds the peptide of SEQ ID NO: 64 or SEQ ID NO: 65;
(ii) inhibits interaction between human ACE2 protein and an S1 protein subunit of SARS-CoV-2;
(iii) inhibits interaction between the carboxypeptidase domain of human ACE2 protein and the S1 protein subunit of SARS-CoV-2;
(iv) inhibits interaction between the carboxypeptidase domain of human ACE2 protein and an RBD of the S1 protein subunit of SARS-CoV-2;
(v) competes for human ACE2-SARS-CoV-2 S1 protein binding;
(vi) binds S1 protein of a SARS-CoV-2 variant and/or the RBD of the S1 protein subunit of the SARS-CoV-2 variant; and
(vii) inhibits a SARS virus infection, optionally, wherein the SARS infection is SARS-CoV-2 infection.

37.-40. (canceled)

41. A method of treating or preventing a coronavirus infection or treating post-acute sequelae of a coronavirus infection in a human subject in need thereof, the method comprising administering to the human subject a therapeutically effective amount of the compound or the pharmaceutically acceptable salt thereof of claim 36.

42.-44. (canceled)

45. A pharmaceutical composition comprising the polypeptide of claim 18, and a pharmaceutically acceptable carrier.

46. A nanoparticle composition comprising the polypeptide of claim 18.

47. A diagnostic reagent for detecting coronavirus whose receptor-binding domain binds to ACE2, the diagnostic reagent comprising a surface comprising the polypeptide of claim 18.

48. A method of detecting the presence of a virus whose receptor binding domain binds to ACE2, the method comprising:
(I) (a) providing a biological sample of a subject;
(b) mixing the biological sample with a plurality of peptides comprising the polypeptide of claim 18 to create a mixture, wherein:
at least one stabilized peptide in the plurality comprises a detection moiety; and
at least one stabilized peptide in the plurality comprises a capture moiety;
(c) providing a diagnostic reagent for detecting the presence of a virus whose receptor-binding domain binds to ACE2;
(d) contacting the diagnostic reagent with the mixture; and
(e) detecting the presence or absence of the virus; or
(II) (a) providing a detection agent, wherein the detection agent is a first polypeptide of claim 18, wherein the first polypeptide binds to the receptor binding domain of the virus, and wherein the first polypeptide is linked to a detection label;
(b) providing a capture agent wherein the capture agent is a second polypeptide of claim 18, wherein the second polypeptide binds to the receptor binding domain of the virus, and wherein the second polypeptide is linked to an affinity label;
(c) mixing a biological sample from a subject with the detection agent and the capture agent to form a mixture;
(d) contacting the mixture with a solid support that binds the capture agent; and
(e) detecting presence or absence of the virus.

49. A method of treating a subject in need thereof, the method comprising administering to the subject a therapeutically effective amount of the polypeptide of claim 18,
wherein the subject is infected with, or at risk of being infected with, a variant of SARS-CoV-2, or
wherein the subject has post-acute sequelae of SARS-CoV-2 infection.

50. A method of preventing or inhibiting interaction between the receptor binding domain of a virus and ACE2 in a subject in need thereof, the method comprising administering to the subject a therapeutically effective amount of the polypeptide of claim 18.

51. A method for treating or preventing a viral infection caused by (i) a coronavirus infection, or (ii) a virus that infects cells by binding to ACE2 or treating post-acute sequelae of SARS-CoV-2 infection in a subject in need thereof, the method comprising administering to a subject in need thereof a therapeutically effective amount of the polypeptide of claim 18.


SEQUENCE LISTING (172 sequences)

SEQ ID NO: 1 - protein, 26 aa, Homo sapiens
IEEQAKTFLDKFNHEAEDLFYQSSLA

SEQ ID NO: 2 - protein, 26 aa, Artificial Sequence
  Synthetic peptide
  MOD_RES (1)..(1): Any non-natural amino acid with an olefinic side chain
  SITE (1)..(5): /note=“May or may not be cross-linked via a side chain”
  SITE (1)..(26): /note=“Variant residues given in the sequence have no preference with respect to those in the annotations for variant positions”
  MOD_RES (5)..(5): Any non-natural amino acid with an olefinic side chain
  VARIANT (25)..(25): /replace=“ ”
  VARIANT (26)..(26): /replace=“ ”
XEEQXKTFLDKFNHEAEDLFYQSSLA

SEQ ID NO: 3 - protein, 26 aa, Artificial Sequence
  Synthetic peptide
  SITE (1)..(26): /note=“Variant residues given in the sequence have no preference with respect to those in the annotations for variant positions”
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  VARIANT (25)..(25): /replace=“ ”
  VARIANT (26)..(26): /replace=“ ”
IEEQXKTFXDKFNHEAEDLFYQSSLA

SEQ ID NO: 4 - protein, 26 aa, Artificial Sequence
  Synthetic peptide
  SITE (1)..(26): /note=“Variant residues given in the sequence have no preference with respect to those in the annotations for variant positions”
  MOD_RES (8)..(8): Any non-natural amino acid with an olefinic side chain
  SITE (8)..(12): /note=“May or may not be cross-linked via a side chain”
  MOD_RES (12)..(12): Any non-natural amino acid with an olefinic side chain
  VARIANT (25)..(25): /replace=“ ”
  VARIANT (26)..(26): /replace=“ ”
IEEQAKTXLDKXNHEAEDLFYQSSLA

SEQ ID NO: 5 - protein, 26 aa, Artificial Sequence
  Synthetic peptide
  SITE (1)..(26): /note=“Variant residues given in the sequence have no preference with respect to those in the annotations for variant positions”
  MOD_RES (12)..(12): Any non-natural amino acid with an olefinic side chain
  SITE (12)..(16): /note=“May or may not be cross-linked via a side chain”
  MOD_RES (16)..(16): Any non-natural amino acid with an olefinic side chain
  VARIANT (25)..(25): /replace=“ ”
  VARIANT (26)..(26): /replace=“ ”
IEEQAKTFLDKXNHEXEDLFYQSSLA

SEQ ID NO: 6 - protein, 26 aa, Artificial Sequence
  Synthetic peptide
  SITE (1)..(26): /note=“Variant residues given in the sequence have no preference with respect to those in the annotations for variant positions”
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
  VARIANT (25)..(25): /replace=“ ”
  VARIANT (26)..(26): /replace=“ ”
IEEQAKTFLDKFNHEXEDLXYQSSLA

SEQ ID NO: 7 - protein, 26 aa, Artificial Sequence
  Synthetic peptide
  SITE (1)..(26): /note=“Variant residues given in the sequence have no preference with respect to those in the annotations for variant positions”
  MOD_RES (9)..(9): Any non-natural amino acid with an olefinic side chain
  SITE (9)..(16): /note=“May or may not be cross-linked via a side chain”
  MOD_RES (16)..(16): Any non-natural amino acid with an olefinic side chain
  VARIANT (25)..(25): /replace=“ ”
  VARIANT (26)..(26): /replace=“ ”
IEEQAKTFXDKFNHEXEDLFYQSSLA

SEQ ID NO: 8 - protein, 26 aa, Artificial Sequence
  Synthetic peptide
  SITE (1)..(26): /note=“Variant residues given in the sequence have no preference with respect to those in the annotations for variant positions”
  MOD_RES (5)..(5): Any non-natural amino acid with an olefinic side chain
  SITE (5)..(12): /note=“May or may not be cross-linked via a side chain”
  MOD_RES (12)..(12): Any non-natural amino acid with an olefinic side chain
  VARIANT (25)..(25): /replace=“ ”
  VARIANT (26)..(26): /replace=“ ”
IEEQXKTFLDKXNHEAEDLFYQSSLA

SEQ ID NO: 9 - protein, 26 aa, Artificial Sequence
  Synthetic peptide
  SITE (1)..(26): /note=“Variant residues given in the sequence have no preference with respect to those in the annotations for variant positions”
  MOD_RES (12)..(12): Any non-natural amino acid with an olefinic side chain
  SITE (12)..(19): /note=“May or may not be cross-linked via a side chain”
  MOD_RES (19)..(19): Any non-natural amino acid with an olefinic side chain
  VARIANT (25)..(25): /replace=“ ”
  VARIANT (26)..(26): /replace=“ ”
IEEQAKTFLDKXNHEAEDXFYQSSLA

SEQ ID NO: 10 - protein, 26 aa, Artificial Sequence
  Synthetic peptide
  MOD_RES (1)..(1): Any non-natural amino acid with an olefinic side chain
  SITE (1)..(5): /note=“May or may not be cross-linked via a side chain”
  SITE (1)..(26): /note=“Variant residues given in the sequence have no preference with respect to those in the annotations for variant positions”
  MOD_RES (5)..(5): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
  VARIANT (25)..(25): /replace=“ ”
  VARIANT (26)..(26): /replace=“ ”
XEEQXKTFLDKFNHEXEDLXYQSSLA

SEQ ID NO: 11 - protein, 26 aa, Artificial Sequence
  Synthetic peptide
  MOD_RES (1)..(1): Any non-natural amino acid with an olefinic side chain
  SITE (1)..(5): /note=“May or may not be cross-linked via a side chain”
  SITE (1)..(26): /note=“Variant residues given in the sequence have no preference with respect to those in the annotations for variant positions”
  MOD_RES (5)..(5): Any non-natural amino acid with an olefinic side chain
  MOD_RES (12)..(12): Any non-natural amino acid with an olefinic side chain
  SITE (12)..(16): /note=“May or may not be cross-linked via a side chain”
  MOD_RES (16)..(16): Any non-natural amino acid with an olefinic side chain
  VARIANT (25)..(25): /replace=“ ”
  VARIANT (26)..(26): /replace=“ ”
XEEQXKTFLDKXNHEXEDLFYQSSLA

SEQ ID NO: 12 - protein, 26 aa, Artificial Sequence
  Synthetic peptide
  SITE (1)..(26): /note=“Variant residues given in the sequence have no preference with respect to those in the annotations for variant positions”
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
  VARIANT (25)..(25): /replace=“ ”
  VARIANT (26)..(26): /replace=“ ”
IEEQXKTFXDKFNHEXEDLXYQSSLA

SEQ ID NO: 13 - protein, 26 aa, Artificial Sequence
  Synthetic peptide
  VARIANT (1)..(1): /replace=“A”
  SITE (1)..(26): /note=“Variant residues given in the sequence have no preference with respect to those in the annotations for variant positions”
  VARIANT (8)..(8): /replace=“A”
  VARIANT (9)..(9): /replace=“A”
  VARIANT (12)..(12): /replace=“A”
  VARIANT (13)..(13): /replace=“A”
  VARIANT (19)..(19): /replace=“A”
  VARIANT (20)..(20): /replace=“A”
  VARIANT (23)..(23): /replace=“A”
  VARIANT (24)..(24): /replace=“A”
  VARIANT (25)..(25): /replace=“A” or “ ”
  VARIANT (26)..(26): /replace=“ ”
IEEQAKTFLDKFNHEAEDLFYQSSLA

SEQ ID NO: 14 - protein, 26 aa, Artificial Sequence
  Synthetic peptide
  SITE (1)..(26): /note=“Variant residues given in the sequence have no preference with respect to those in the annotations for variant positions”
  VARIANT (22)..(22): /replace=“Y”
  VARIANT (25)..(25): /replace=“A” or “ ”
  VARIANT (26)..(26): /replace=“ ”
IEEQAKTFLDKFNHEAEDLFYQSSLA

SEQ ID NO: 15 - protein, 26 aa, Artificial Sequence
  Synthetic peptide
  SITE (1)..(26): /note=“Variant residues given in the sequence have no preference with respect to those in the annotations for variant positions”
  VARIANT (7)..(7): /replace=“E”
  VARIANT (25)..(25): /replace=“A” or “ ”
  VARIANT (26)..(26): /replace=“ ”
IEEQAKTFLDKFNHEAEDLFYQSSLA

SEQ ID NO: 16 - protein, 26 aa, Artificial Sequence
  Synthetic peptide
  SITE (1)..(26): /note=“Variant residues given in the sequence have no preference with respect to those in the annotations for variant positions”
  VARIANT (7)..(7): /replace=“F”
  VARIANT (25)..(25): /replace=“A” or “ ”
  VARIANT (26)..(26): /replace=“ ”
IEEQAKTFLDKFNHEAEDLFYQSSLA

SEQ ID NO: 17 - protein, 26 aa, Artificial Sequence
  Synthetic peptide
  VARIANT (1)..(1): /replace=“A” or “Any stapling amino acid”
  SITE (1)..(26): /note=“Variant residues given in the sequence have no preference with respect to those in the annotations for variant positions”
  VARIANT (5)..(5): /replace=“Any stapling amino acid”
  VARIANT (7)..(7): /replace=“E” or “F”
  VARIANT (8)..(8): /replace=“A” or “Any stapling amino acid”
  VARIANT (9)..(9): /replace=“A” or “Any stapling amino acid”
  VARIANT (12)..(12): /replace=“A” or “Any stapling amino acid”
  VARIANT (13)..(13): /replace=“A”
  VARIANT (16)..(16): /replace=“Any stapling amino acid”
  VARIANT (19)..(19): /replace=“A” or “Any stapling amino acid”
  VARIANT (20)..(20): /replace=“A” or “Any stapling amino acid”
  VARIANT (22)..(22): /replace=“Y”
  VARIANT (23)..(23): /replace=“A”
  VARIANT (24)..(24): /replace=“A”
  VARIANT (25)..(25): /replace=“A” or “ ”
  VARIANT (26)..(26): /replace=“ ”
IEEQAKTFLDKFNHEAEDLFYQSSLA

SEQ ID NO: 18 - protein, 26 aa, Artificial Sequence
  Synthetic peptide
  VARIANT (1)..(1): /replace=“A” or “Any stapling amino acid”
  SITE (1)..(26): /note=“Variant residues given in the sequence have no preference with respect to those in the annotations for variant positions”
  VARIANT (2)..(2): /replace=“A”
  VARIANT (5)..(5): /replace=“Any stapling amino acid”
  VARIANT (6)..(6): /replace=“R” or “A”
  VARIANT (8)..(8): /replace=“A” or “Any stapling amino acid”
  VARIANT (9)..(9): /replace=“A” or “Any stapling amino acid”
  VARIANT (12)..(12): /replace=“A” or “Any stapling amino acid”
  VARIANT (13)..(13): /replace=“A”
  VARIANT (16)..(16): /replace=“Any stapling amino acid”
  VARIANT (17)..(17): /replace=“D” or “A”
  VARIANT (18)..(18): /replace=“E” or “A”
  VARIANT (19)..(19): /replace=“A” or “Any stapling amino acid”
  VARIANT (20)..(20): /replace=“A” or “Any stapling amino acid”
  VARIANT (23)..(23): /replace=“A”
  VARIANT (24)..(24): /replace=“A”
  VARIANT (26)..(26): /replace=“ ”
IEEQAKTFLDKFNHEAEDLFYQSSLA

SEQ ID NO: 19 - protein, 26 aa, Artificial Sequence
  Synthetic peptide
  SITE (6)..(10): /note=“Linked by a lactam bridge”
  SITE (11)..(18): /note=“Linked by a lactam bridge”
IEEQAKTFLDKFNHEAEDLFYQSSLA

SEQ ID NO: 20 - protein, 26 aa, Artificial Sequence
  Synthetic peptide
  VARIANT (1)..(1): /replace=“A”
  SITE (1)..(26): /note=“Variant residues given in the sequence have no preference with respect to those in the annotations for variant positions”
  SITE (6)..(10): /note=“Linked by a lactam bridge”
  VARIANT (7)..(7): /replace=“E” or “F”
  VARIANT (8)..(8): /replace=“A”
  VARIANT (9)..(9): /replace=“A”
  SITE (11)..(18): /note=“Linked by a lactam bridge”
  VARIANT (12)..(12): /replace=“A”
  VARIANT (13)..(13): /replace=“A”
  VARIANT (19)..(19): /replace=“A”
  VARIANT (20)..(20): /replace=“A”
  VARIANT (22)..(22): /replace=“Y”
  VARIANT (23)..(23): /replace=“A”
  VARIANT (24)..(24): /replace=“A”
  VARIANT (25)..(25): /replace=“A”
  VARIANT (26)..(26): /replace=“ ”
IEEQAKTFLDKFNHEAEDLFYQSSLA

SEQ ID NO: 21 - protein, 24 aa, Homo sapiens
IEEQAKTFLDKFNHEAEDLFYQSS

SEQ ID NO: 22 - protein, 805 aa, Homo sapiens [sequence omitted]

SEQ ID NO: 23 - protein, 21 aa, Artificial Sequence
  Synthetic peptide
  SITE (1)..(21): /note=“Variant residues given in the sequence have no preference with respect to those in the annotations for variant positions”
  VARIANT (13)..(13): /replace=“Q”
  VARIANT (20)..(20): /replace=“ ”
  VARIANT (21)..(21): /replace=“ ”
KTFLDKFNHEAEDLFYQSSLA

SEQ ID NO: 24 - protein, 4 aa, Artificial Sequence
  Synthetic peptide
IEEQ

SEQ ID NO: 25 - protein, 17 aa, Artificial Sequence
  Synthetic peptide
  SITE (1)..(17): /note=“Variant residues given in the sequence have no preference with respect to those in the annotations for variant positions”
  VARIANT (9)..(9): /replace=“Q”
  VARIANT (16)..(16): /replace=“ ”
  VARIANT (17)..(17): /replace=“ ”
DKFNHEAEDLFYQSSLA

SEQ ID NO: 26 - protein, 7 aa, Artificial Sequence
  Synthetic peptide
IEEQAKT

SEQ ID NO: 27 - skipped

SEQ ID NO: 28 - protein, 14 aa, Artificial Sequence
  Synthetic peptide
  SITE (1)..(14): /note=“Variant residues given in the sequence have no preference with respect to those in the annotations for variant positions”
  VARIANT (6)..(6): /replace=“Q”
  VARIANT (13)..(13): /replace=“ ”
  VARIANT (14)..(14): /replace=“ ”
NHEAEDLFYQSSLA

SEQ ID NO: 29 - protein, 11 aa, Artificial Sequence
  Synthetic peptide
IEEQAKTFLDK

SEQ ID NO: 30 - skipped

SEQ ID NO: 31 - protein, 10 aa, Artificial Sequence
  Synthetic peptide
  SITE (1)..(10): /note=“Variant residues given in the sequence have no preference with respect to those in the annotations for variant positions”
  VARIANT (2)..(2): /replace=“Q”
  VARIANT (9)..(9): /replace=“ ”
  VARIANT (10)..(10): /replace=“ ”
EDLFYQSSLA

SEQ ID NO: 32 - protein, 15 aa, Artificial Sequence
  Synthetic peptide
IEEQAKTFLDKFNHE

SEQ ID NO: 33 - protein, 6 aa, Artificial Sequence
  Synthetic peptide
  SITE (1)..(6): /note=“Variant residues given in the sequence have no preference with respect to those in the annotations for variant positions”
  VARIANT (5)..(5): /replace=“ ”
  VARIANT (6)..(6): /replace=“ ”
YQSSLA

SEQ ID NO: 34 - protein, 8 aa, Artificial Sequence
  Synthetic peptide
IEEQAKTF

SEQ ID NO: 35 - protein, 6 aa, Artificial Sequence
  Synthetic peptide
DKFNHE

SEQ ID NO: 36 - protein, 10 aa, Artificial Sequence
  Synthetic peptide
  SITE (1)..(10): /note=“Variant residues given in the sequence have no preference with respect to those in the annotations for variant positions”
  VARIANT (2)..(2): /replace=“Q”
  VARIANT (9)..(9): /replace=“ ”
  VARIANT (10)..(10): /replace=“ ”
EDLFYQSSLA

SEQ ID NO: 37 - skipped

SEQ ID NO: 38 - protein, 6 aa, Artificial Sequence
  Synthetic peptide
KTFLDK

SEQ ID NO: 39 - protein, 14 aa, Artificial Sequence
  Synthetic peptide
  SITE (1)..(14): /note=“Variant residues given in the sequence have no preference with respect to those in the annotations for variant positions”
  VARIANT (6)..(6): /replace=“Q”
  VARIANT (13)..(13): /replace=“ ”
  VARIANT (14)..(14): /replace=“ ”
NHEAEDLFYQSSLA

SEQ ID NO: 40 - skipped

SEQ ID NO: 41 - protein, 6 aa, Artificial Sequence
  Synthetic peptide
  SITE (1)..(6): /note=“Variant residues given in the sequence have no preference with respect to those in the annotations for variant positions”
  VARIANT (6)..(6): /replace=“Q”
NHEAED

SEQ ID NO: 42 - protein, 7 aa, Artificial Sequence
  Synthetic peptide
  SITE (1)..(7): /note=“Variant residues given in the sequence have no preference with respect to those in the annotations for variant positions”
  VARIANT (6)..(6): /replace=“ ”
  VARIANT (7)..(7): /replace=“ ”
FYQSSLA

SEQ ID NO: 43 - protein, 10 aa, Artificial Sequence
  Synthetic peptide
KTFLDKFNHE

SEQ ID NO: 44 - protein, 6 aa, Artificial Sequence
  Synthetic peptide
  SITE (1)..(6): /note=“Variant residues given in the sequence have no preference with respect to those in the annotations for variant positions”
  VARIANT (5)..(5): /replace=“ ”
  VARIANT (6)..(6): /replace=“ ”
YQSSLA

SEQ ID NO: 45 - skipped

SEQ ID NO: 46 - protein, 10 aa, Artificial Sequence
  Synthetic peptide
  SITE (1)..(10): /note=“Variant residues given in the sequence have no preference with respect to those in the annotations for variant positions”
  VARIANT (9)..(9): /replace=“ ”
  VARIANT (10)..(10): /replace=“ ”
EDLFYQSSLA

SEQ ID NO: 47 - skipped

SEQ ID NO: 48 - protein, 6 aa, Artificial Sequence
  Synthetic peptide
DKFNHE

SEQ ID NO: 49 - protein, 26 aa, Artificial Sequence
  Synthetic peptide
  SITE (1)..(26): /note=“Variant residues given in the sequence have no preference with respect to those in the annotations for variant positions”
  VARIANT (24)..(24): /replace=“ ”
  VARIANT (25)..(25): /replace=“A” or “ ”
  VARIANT (26)..(26): /replace=“ ”
IEEQAKTFLDKFNHEAEDLFYQSALA

SEQ ID NO: 50 - protein, 26 aa, Artificial Sequence
  Synthetic peptide
  SITE (1)..(26): /note=“Variant residues given in the sequence have no preference with respect to those in the annotations for variant positions”
  VARIANT (25)..(25): /replace=“A” or “ ”
  VARIANT (26)..(26): /replace=“ ”
IEEQAKTAADKANHEAEDAAYQSALA

SEQ ID NO: 51 - protein, 26 aa, Artificial Sequence
  Synthetic peptide
  SITE (1)..(26): /note=“Variant residues given in the sequence have no preference with respect to those in the annotations for variant positions”
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  VARIANT (25)..(25): /replace=“A” or “ ”
  VARIANT (26)..(26): /replace=“ ”
IEEQXKTAXDKANHEAEDAAYQSALA

SEQ ID NO: 52 - protein, 26 aa, Artificial Sequence
  Synthetic peptide
  SITE (1)..(26): /note=“Variant residues given in the sequence have no preference with respect to those in the annotations for variant positions”
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
  VARIANT (25)..(25): /replace=“A” or “ ”
  VARIANT (26)..(26): /replace=“ ”
IEEQXKTAXDKANHEXEDAXYQSALA

SEQ ID NO: 53 - protein, 26 aa, Artificial Sequence
  Synthetic peptide
  SITE (1)..(26): /note=“Variant residues given in the sequence have no preference with respect to those in the annotations for variant positions”
  VARIANT (25)..(25): /replace=“A” or “ ”
  VARIANT (26)..(26): /replace=“ ”
IQEQAKTDADKHNHEAEDYQYQSALA

SEQ ID NO: 54 - protein, 26 aa, Artificial Sequence
  Synthetic peptide
  SITE (1)..(26): /note=“Variant residues given in the sequence have no preference with respect to those in the annotations for variant positions”
  VARIANT (25)..(25): /replace=“A” or “ ”
  VARIANT (26)..(26): /replace=“ ”
QEEQAKDAADHANHEAEYQAYQSALA

SEQ ID NO: 55 - protein, 26 aa, Artificial Sequence
  Synthetic peptide
  SITE (1)..(26): /note=“Variant residues given in the sequence have no preference with respect to those in the annotations for variant positions”
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
  VARIANT (25)..(25): /replace=“A” or “ ”
  VARIANT (26)..(26): /replace=“ ”
QEEQXKDAXDHANHEXEYQXYQSALA

SEQ ID NO: 56 - protein, 26 aa, Artificial Sequence
  Synthetic peptide
  SITE (1)..(26): /note=“Variant residues given in the sequence have no preference with respect to those in the annotations for variant positions”
  VARIANT (25)..(25): /replace=“A” or “ ”
  VARIANT (26)..(26): /replace=“ ”
IEEQAKTAADKANHEAEQAAYQSALA

SEQ ID NO: 57 - protein, 26 aa, Artificial Sequence
  Synthetic peptide
  SITE (1)..(26): /note=“Variant residues given in the sequence have no preference with respect to those in the annotations for variant positions”
  MOD_RES (8)..(8): Any non-natural amino acid with an olefinic side chain
  SITE (8)..(12): /note=“May or may not be cross-linked via a side chain”
  MOD_RES (12)..(12): Any non-natural amino acid with an olefinic side chain
  VARIANT (25)..(25): /replace=“A” or “ ”
  VARIANT (26)..(26): /replace=“ ”
IEEQAKTXADKXNHEAEQAAYQSALA

SEQ ID NO: 58 - protein, 26 aa, Artificial Sequence
  Synthetic peptide
  SITE (1)..(26): /note=“Variant residues given in the sequence have no preference with respect to those in the annotations for variant positions”
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
  VARIANT (25)..(25): /replace=“A” or “ ”
  VARIANT (26)..(26): /replace=“ ”
IEEQXKTAXDKANHEXEQAXYQSALA

SEQ ID NO: 59 - protein, 26 aa, Artificial Sequence
  Synthetic peptide
  SITE (1)..(26): /note=“Variant residues given in the sequence have no preference with respect to those in the annotations for variant positions”
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
  VARIANT (25)..(25): /replace=“A” or “ ”
  VARIANT (26)..(26): /replace=“ ”
IEEQXKEAXDKANHEXEQAXYQSALA

SEQ ID NO: 60 - protein, 26 aa, Artificial Sequence
  Synthetic peptide
  SITE (1)..(26): /note=“Variant residues given in the sequence have no preference with respect to those in the annotations for variant positions”
  MOD_RES (9)..(9): Any non-natural amino acid with an olefinic side chain
  SITE (9)..(16): /note=“May or may not be cross-linked via a side chain”
  MOD_RES (16)..(16): Any non-natural amino acid with an olefinic side chain
  VARIANT (25)..(25): /replace=“A” or “ ”
  VARIANT (26)..(26): /replace=“ ”
IEEQAKTAXDKANHEXEQAAYQSALA

SEQ ID NO: 61 - protein, 8 aa, Artificial Sequence
  Synthetic peptide
IEEQAKTA

SEQ ID NO: 62 - protein, 6 aa, Artificial Sequence
  Synthetic peptide
DKANHE

SEQ ID NO: 63 - protein, 6 aa, Artificial Sequence
  Synthetic peptide
  SITE (1)..(6): /note=“Variant residues given in the sequence have no preference with respect to those in the annotations for variant positions”
  VARIANT (5)..(5): /replace=“A” or “ ”
  VARIANT (6)..(6): /replace=“ ”
YQSALA

SEQ ID NO: 64 - protein, 223 aa, Artificial Sequence
  Receptor Binding Motif sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 65 - protein, 190 aa, Artificial Sequence
  Receptor Binding Motif sequence
FPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELL

SEQ ID NO: 66 - protein, 80 aa, Homo sapiens
MSSSSWLLLSLVAVTAAQSTIEEQAKTFLDKFNHEAEDLFYQSSLASWNYNTNITEENVQNMNNAGDKWSAFLKEQSTLA

SEQ ID NO: 67 - protein, 17 aa, Artificial Sequence
  Synthetic peptide
  SITE (1)..(17): /note=“Variant residues given in the sequence have no preference with respect to those in the annotations for variant positions”
  VARIANT (9)..(9): /replace=“Q”
  VARIANT (16)..(16): /replace=“A” or “ ”
  VARIANT (17)..(17): /replace=“ ”
DKANHEAEDAAYQSALA

SEQ ID NO: 68 - protein, 14 aa, Artificial Sequence
  Synthetic peptide
  SITE (1)..(14): /note=“Variant residues given in the sequence have no preference with respect to those in the annotations for variant positions”
  VARIANT (13)..(13): /replace=“A” or “ ”
  VARIANT (14)..(14): /replace=“ ”
NHEAEQAAYQSALA

SEQ ID NO: 69 - protein, 10 aa, Artificial Sequence
  Synthetic peptide
  SITE (1)..(10): /note=“Variant residues given in the sequence have no preference with respect to those in the annotations for variant positions”
  VARIANT (9)..(9): /replace=“A” or “ ”
  VARIANT (10)..(10): /replace=“ ”
EQAAYQSALA

SEQ ID NO: 70 - protein, 4 aa, Artificial Sequence
  Synthetic peptide
QEEQ

SEQ ID NO: 71 - protein, 80 aa, Bos taurus
MTGSFWLLLSLVAVTAAQSTTEEQAKTFLEKFNHEAEDLSYQSSLASWNYNTNITDENVQKMNEARAKWSAFYEEQSRMA

SEQ ID NO: 72 - protein, 80 aa, Pan troglodytes
MSGSSWLLLSLVAVTAAQSTIEEQAKTFLDKFNHEAEDLFYQSSLASWNYNTNITEENVQNMNNAGDKWSAFLKEQSTLA

SEQ ID NO: 73 - protein, 80 aa, Macaca mulatta
MSGSSWLLLSLVAVTAAQSTIEEQAKTFLDKFNHEAEDLFYQSSLASWNYNTNITEENVQNMNNAGEKWSAFLKEQSTLA

SEQ ID NO: 74 - protein, 79 aa, Canis lupus familiaris
MSGSSWLLLSLAALTAAQSTEDLVKTFLEKFNYEAEELSYQSSLASWNYNINITDENVQKMNNAGAKWSAFYEEQSKLA

SEQ ID NO: 75 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 76 - protein, 23 aa, Homo sapiens
IEEQAKTFLDKFNHEAEDLFYQS

SEQ ID NO: 77 - protein, 20 aa, Homo sapiens
EQAKTFLDKFNHEAEDLFYQ

SEQ ID NO: 78 - protein, 23 aa, Artificial Sequence
  Synthetic peptide
  MOD_RES (1)..(1): Any non-natural amino acid with an olefinic side chain
  SITE (1)..(5): /note=“May or may not be cross-linked via a side chain”
  MOD_RES (5)..(5): Any non-natural amino acid with an olefinic side chain
XEEQXKTFLDKFNHEAEDLFYQS

SEQ ID NO: 79 - protein, 23 aa, Artificial Sequence
  Synthetic peptide
  MOD_RES (2)..(2): Any non-natural amino acid with an olefinic side chain
  SITE (2)..(6): /note=“May or may not be cross-linked via a side chain”
  MOD_RES (6)..(6): Any non-natural amino acid with an olefinic side chain
IXEQAXTFLDKFNHEAEDLFYQS

SEQ ID NO: 80 - protein, 23 aa, Artificial Sequence
  Synthetic peptide
  MOD_RES (3)..(3): Any non-natural amino acid with an olefinic side chain
  SITE (3)..(7): /note=“May or may not be cross-linked via a side chain”
  MOD_RES (7)..(7): Any non-natural amino acid with an olefinic side chain
IEXQAKXFLDKFNHEAEDLFYQS

SEQ ID NO: 81 - protein, 23 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
IEEQXKTFXDKFNHEAEDLFYQS

SEQ ID NO: 82 - protein, 23 aa, Artificial Sequence
  Synthetic peptide
  MOD_RES (6)..(6): Any non-natural amino acid with an olefinic side chain
  SITE (6)..(10): /note=“May or may not be cross-linked via a side chain”
  MOD_RES (10)..(10): Any non-natural amino acid with an olefinic side chain
IEEQAXTFLXKFNHEAEDLFYQS

SEQ ID NO: 83 - protein, 23 aa, Artificial Sequence
  Synthetic peptide
  MOD_RES (7)..(7): Any non-natural amino acid with an olefinic side chain
  SITE (7)..(11): /note=“May or may not be cross-linked via a side chain”
  MOD_RES (11)..(11): Any non-natural amino acid with an olefinic side chain
IEEQAKXFLDXFNHEAEDLFYQS

SEQ ID NO: 84 - protein, 23 aa, Artificial Sequence
  Synthetic peptide
  MOD_RES (8)..(8): Any non-natural amino acid with an olefinic side chain
  SITE (8)..(12): /note=“May or may not be cross-linked via a side chain”
  MOD_RES (12)..(12): Any non-natural amino acid with an olefinic side chain
IEEQAKTXLDKXNHEAEDLFYQS

SEQ ID NO: 85 - protein, 23 aa, Artificial Sequence
  Synthetic peptide
  MOD_RES (9)..(9): Any non-natural amino acid with an olefinic side chain
  SITE (9)..(13): /note=“May or may not be cross-linked via a side chain”
  MOD_RES (13)..(13): Any non-natural amino acid with an olefinic side chain
IEEQAKTFXDKFXHEAEDLFYQS

SEQ ID NO: 86 - protein, 23 aa, Artificial Sequence
  Synthetic peptide
  MOD_RES (10)..(10): Any non-natural amino acid with an olefinic side chain
  SITE (10)..(14): /note=“May or may not be cross-linked via a side chain”
  MOD_RES (14)..(14): Any non-natural amino acid with an olefinic side chain
IEEQAKTFLXKFNXEAEDLFYQS

SEQ ID NO: 87 - protein, 23 aa, Artificial Sequence
  Synthetic peptide
  MOD_RES (11)..(11): Any non-natural amino acid with an olefinic side chain
  SITE (11)..(15): /note=“May or may not be cross-linked via a side chain”
  MOD_RES (15)..(15): Any non-natural amino acid with an olefinic side chain
IEEQAKTFLDXFNHXAEDLFYQS

SEQ ID NO: 88 - protein, 23 aa, Artificial Sequence
  Synthetic peptide
  MOD_RES (12)..(12): Any non-natural amino acid with an olefinic side chain
  SITE (12)..(16): /note=“May or may not be cross-linked via a side chain”
  MOD_RES (16)..(16): Any non-natural amino acid with an olefinic side chain
IEEQAKTFLDKXNHEXEDLFYQS

SEQ ID NO: 89 - protein, 23 aa, Artificial Sequence
  Synthetic peptide
  MOD_RES (13)..(13): Any non-natural amino acid with an olefinic side chain
  SITE (13)..(17): /note=“May or may not be cross-linked via a side chain”
  MOD_RES (17)..(17): Any non-natural amino acid with an olefinic side chain
IEEQAKTFLDKFXHEAXDLFYQS

SEQ ID NO: 90 - protein, 23 aa, Artificial Sequence
  Synthetic peptide
  MOD_RES (14)..(14): Any non-natural amino acid with an olefinic side chain
  SITE (14)..(18): /note=“May or may not be cross-linked via a side chain”
  MOD_RES (18)..(18): Any non-natural amino acid with an olefinic side chain
IEEQAKTFLDKFNXEAEXLFYQS

SEQ ID NO: 91 - protein, 23 aa, Artificial Sequence
  Synthetic peptide
  MOD_RES (15)..(15): Any non-natural amino acid with an olefinic side chain
  SITE (15)..(19): /note=“May or may not be cross-linked via a side chain”
  MOD_RES (19)..(19): Any non-natural amino acid with an olefinic side chain
IEEQAKTFLDKFNHXAEDXFYQS

SEQ ID NO: 92 - protein, 23 aa, Artificial Sequence
  Synthetic peptide
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
IEEQAKTFLDKFNHEXEDLXYQS

SEQ ID NO: 93 - protein, 23 aa, Artificial Sequence
  Synthetic peptide
  MOD_RES (17)..(17): Any non-natural amino acid with an olefinic side chain
  SITE (17)..(21): /note=“May or may not be cross-linked via a side chain”
  MOD_RES (21)..(21): Any non-natural amino acid with an olefinic side chain
IEEQAKTFLDKFNHEAXDLFXQS

SEQ ID NO: 94 - protein, 23 aa, Artificial Sequence
  Synthetic peptide
  MOD_RES (18)..(18): Any non-natural amino acid with an olefinic side chain
  SITE (18)..(22): /note=“May or may not be cross-linked via a side chain”
  MOD_RES (22)..(22): Any non-natural amino acid with an olefinic side chain
IEEQAKTFLDKFNHEAEXLFYXS

SEQ ID NO: 95 - protein, 23 aa, Artificial Sequence
  Synthetic peptide
  MOD_RES (19)..(19): Any non-natural amino acid with an olefinic side chain
  SITE (19)..(23): /note=“May or may not be cross-linked via a side chain”
  MOD_RES (23)..(23): Any non-natural amino acid with an olefinic side chain
IEEQAKTFLDKFNHEAEDXFYQX

SEQ ID NO: 96 - protein, 23 aa, Artificial Sequence
  Synthetic peptide
  MOD_RES (1)..(1): Any non-natural amino acid with an olefinic side chain
  SITE (1)..(8): /note=“May or may not be cross-linked via a side chain”
  MOD_RES (8)..(8): Any non-natural amino acid with an olefinic side chain
XEEQAKTXLDKFNHEAEDLFYQS

SEQ ID NO: 97 - protein, 23 aa, Artificial Sequence
  Synthetic peptide
  MOD_RES (2)..(2): Any non-natural amino acid with an olefinic side chain
  SITE (2)..(9): /note=“May or may not be cross-linked via a side chain”
  MOD_RES (9)..(9): Any non-natural amino acid with an olefinic side chain
IXEQAKTFXDKFNHEAEDLFYQS

SEQ ID NO: 98 - protein, 23 aa, Artificial Sequence
  Synthetic peptide
  MOD_RES (3)..(3): Any non-natural amino acid with an olefinic side chain
  SITE (3)..(10): /note=“May or may not be cross-linked via a side chain”
  MOD_RES (10)..(10): Any non-natural amino acid with an olefinic side chain
IEXQAKTFLXKFNHEAEDLFYQS

SEQ ID NO: 99 - protein, 23 aa, Artificial Sequence
  Synthetic peptide
  MOD_RES (4)..(4): Any non-natural amino acid with an olefinic side chain
  SITE (4)..(11): /note=“May or may not be cross-linked via a side chain”
  MOD_RES (11)..(11): Any non-natural amino acid with an olefinic side chain
IEEXAKTFLDXFNHEAEDLFYQS

SEQ ID NO: 100 - protein, 23 aa, Artificial Sequence
  Synthetic peptide
  MOD_RES (5)..(5): Any non-natural amino acid with an olefinic side chain
  SITE (5)..(12): /note=“May or may not be cross-linked via a side chain”
  MOD_RES (12)..(12): Any non-natural amino acid with an olefinic side chain
IEEQXKTFLDKXNHEAEDLFYQS

SEQ ID NO: 101 - protein, 23 aa, Artificial Sequence
  Synthetic peptide
  MOD_RES (6)..(6): Any non-natural amino acid with an olefinic side chain
  SITE (6)..(13): /note=“May or may not be cross-linked via a side chain”
  MOD_RES (13)..(13): Any non-natural amino acid with an olefinic side chain
IEEQAXTFLDKFXHEAEDLFYQS

SEQ ID NO: 102 - protein, 23 aa, Artificial Sequence
  Synthetic peptide
  MOD_RES (7)..(7): Any non-natural amino acid with an olefinic side chain
  SITE (7)..(14): /note=“May or may not be cross-linked via a side chain”
  MOD_RES (14)..(14): Any non-natural amino acid with an olefinic side chain
IEEQAKXFLDKFNXEAEDLFYQS

SEQ ID NO: 103 - protein, 23 aa, Artificial Sequence
  Synthetic peptide
  MOD_RES (8)..(8): Any non-natural amino acid with an olefinic side chain
  SITE (8)..(15): /note=“May or may not be cross-linked via a side chain”
  MOD_RES (15)..(15): Any non-natural amino acid with an olefinic side chain
IEEQAKTXLDKFNHXAEDLFYQS

SEQ ID NO: 104 - protein, 23 aa, Artificial Sequence
  Synthetic peptide
  MOD_RES (9)..(9): Any non-natural amino acid with an olefinic side chain
  SITE (9)..(16): /note=“May or may not be cross-linked via a side chain”
  MOD_RES (16)..(16): Any non-natural amino acid with an olefinic side chain
IEEQAKTFXDKFNHEXEDLFYQS

SEQ ID NO: 105 - protein, 23 aa, Artificial Sequence
  Synthetic peptide
  MOD_RES (10)..(10): Any non-natural amino acid with an olefinic side chain
  SITE (10)..(17): /note=“May or may not be cross-linked via a side chain”
  MOD_RES (17)..(17): Any non-natural amino acid with an olefinic side chain
IEEQAKTFLXKFNHEAXDLFYQS

SEQ ID NO: 106 - protein, 23 aa, Artificial Sequence
  Synthetic peptide
  MOD_RES (11)..(11): Any non-natural amino acid with an olefinic side chain
  SITE (11)..(18): /note=“May or may not be cross-linked via a side chain”
  MOD_RES (18)..(18): Any non-natural amino acid with an olefinic side chain
IEEQAKTFLDXFNHEAEXLFYQS

SEQ ID NO: 107 - protein, 23 aa, Artificial Sequence
  Synthetic peptide
  MOD_RES (12)..(12): Any non-natural amino acid with an olefinic side chain
  SITE (12)..(19): /note=“May or may not be cross-linked via a side chain”
  MOD_RES (19)..(19): Any non-natural amino acid with an olefinic side chain
IEEQAKTFLDKXNHEAEDXFYQS

SEQ ID NO: 108 - protein, 23 aa, Artificial Sequence
  Synthetic peptide
  MOD_RES (13)..(13): Any non-natural amino acid with an olefinic side chain
  SITE (13)..(20): /note=“May or may not be cross-linked via a side chain”
  MOD_RES (20)..(20): Any non-natural amino acid with an olefinic side chain
IEEQAKTFLDKFXHEAEDLXYQS

SEQ ID NO: 109 - protein, 23 aa, Artificial Sequence
  Synthetic peptide
  MOD_RES (14)..(14): Any non-natural amino acid with an olefinic side chain
  SITE (14)..(21): /note=“May or may not be cross-linked via a side chain”
  MOD_RES (21)..(21): Any non-natural amino acid with an olefinic side chain
IEEQAKTFLDKFNXEAEDLFXQS

SEQ ID NO: 110 - protein, 23 aa, Artificial Sequence
  Synthetic peptide
  MOD_RES (15)..(15): Any non-natural amino acid with an olefinic side chain
  SITE (15)..(22): /note=“May or may not be cross-linked via a side chain”
  MOD_RES (22)..(22): Any non-natural amino acid with an olefinic side chain
IEEQAKTFLDKFNHXAEDLFYXS

SEQ ID NO: 111 - protein, 23 aa, Artificial Sequence
  Synthetic peptide
  MOD_RES (16)..(16): Any non-natural amino acid with an olefinic side chain
  SITE (16)..(23): /note=“May or may not be cross-linked via a side chain”
  MOD_RES (23)..(23): Any non-natural amino acid with an olefinic side chain
IEEQAKTFLDKFNHEXEDLFYQX

SEQ ID NO: 112 - protein, 24 aa, Artificial Sequence
  Synthetic peptide
IEEQAKTFLDKFNHEAEDLFYQSA

SEQ ID NO: 113 - protein, 24 aa, Artificial Sequence
  Synthetic peptide
IEEQAKTAADKANHEAEDAAYQSA

SEQ ID NO: 114 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
IEEQXKTAXDKANHEAEDAAYQSA

SEQ ID NO: 115 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
IEEQXKTAXDKANHEXEDAXYQSA

SEQ ID NO: 116 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
IEEQXKEAXDKANHEXEDAXYQSA

SEQ ID NO: 117 - protein, 24 aa, Artificial Sequence
  Synthetic peptide
AEEQAKTAADKAAHEAEQAAYQAA

SEQ ID NO: 118 - protein, 24 aa, Artificial Sequence
  Synthetic peptide
IEEQAKTAADKANHEAEQAAYQSA

SEQ ID NO: 119 - protein, 24 aa, Artificial Sequence
  Synthetic peptide
  MOD_RES (8)..(8): Any non-natural amino acid with an olefinic side chain
  SITE (8)..(12): /note=“May or may not be cross-linked via a side chain”
  MOD_RES (12)..(12): Any non-natural amino acid with an olefinic side chain
IEEQAKTXADKXNHEAEQAAYQSA

SEQ ID NO: 120 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
IEEQXKTAXDKANHEXEQAXYQSA

SEQ ID NO: 121 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
IEEQXKEAXDKANHEXEQAXYQSA

SEQ ID NO: 122 - protein, 24 aa, Artificial Sequence
  Synthetic peptide
  MOD_RES (9)..(9): Any non-natural amino acid with an olefinic side chain
  SITE (9)..(16): /note=“May or may not be cross-linked via a side chain”
  MOD_RES (16)..(16): Any non-natural amino acid with an olefinic side chain
IEEQAKTAXDKANHEXEQAAYQSA

SEQ ID NO: 123 - protein, 24 aa, Artificial Sequence
  Synthetic peptide
IQEQAKTDADKHNHEAEDYQYQSA

SEQ ID NO: 124 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
IQEQXKTDXDKHNHEAEDYQYQSA

SEQ ID NO: 125 - protein, 24 aa, Artificial Sequence
  Synthetic peptide
QEEQAKDAADHANHEAEYQAYQSA

SEQ ID NO: 126 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
QEEQXKDAXDHANHEXEYQXYQSA

SEQ ID NO: 127 - protein, 20 aa, Artificial Sequence
  Synthetic peptide
ETVDFFAEWFDVEAEDKDYL

SEQ ID NO: 128 - protein, 20 aa, Artificial Sequence
  Synthetic peptide
  MOD_RES (3)..(3): Any non-natural amino acid with an olefinic side chain
  SITE (3)..(7): /note=“May or may not be cross-linked via a side chain”
  MOD_RES (7)..(7): Any non-natural amino acid with an olefinic side chain
  MOD_RES (13)..(13): Any non-natural amino acid with an olefinic side chain
  SITE (13)..(17): /note=“May or may not be cross-linked via a side chain”
  MOD_RES (17)..(17): Any non-natural amino acid with an olefinic side chain
ETXDFFXEWADVXAEDXDYL

SEQ ID NO: 129 - protein, 20 aa, Artificial Sequence
  Synthetic peptide
  MOD_RES (3)..(3): Any non-natural amino acid with an olefinic side chain
  SITE (3)..(7): /note=“May or may not be cross-linked via a side chain”
  MOD_RES (7)..(7): Any non-natural amino acid with an olefinic side chain
  MOD_RES (14)..(14): Any non-natural amino acid with an olefinic side chain
  SITE (14)..(18): /note=“May or may not be cross-linked via a side chain”
  MOD_RES (18)..(18): Any non-natural amino acid with an olefinic side chain
ETXDFFXEWADVEXEDKXYL

SEQ ID NO: 130 - protein, 20 aa, Artificial Sequence
  Synthetic peptide
  MOD_RES (3)..(3): Any non-natural amino acid with an olefinic side chain
  SITE (3)..(7): /note=“May or may not be cross-linked via a side chain”
  MOD_RES (7)..(7): Any non-natural amino acid with an olefinic side chain
  MOD_RES (13)..(13): Any non-natural amino acid with an olefinic side chain
  SITE (13)..(17): /note=“May or may not be cross-linked via a side chain”
  MOD_RES (17)..(17): Any non-natural amino acid with an olefinic side chain
ETXDFFXEWFDVXAEDXDYL

SEQ ID NO: 131 - protein, 20 aa, Artificial Sequence
  Synthetic peptide
  MOD_RES (3)..(3): Any non-natural amino acid with an olefinic side chain
  SITE (3)..(7): /note=“May or may not be cross-linked via a side chain”
  MOD_RES (7)..(7): Any non-natural amino acid with an olefinic side chain
  MOD_RES (13)..(13): Any non-natural amino acid with an olefinic side chain
  SITE (13)..(17): /note=“May or may not be cross-linked via a side chain”
  MOD_RES (17)..(17): Any non-natural amino acid with an olefinic side chain
ETXDFEXEWFDVXAEDXDYL

SEQ ID NO: 132 - protein, 20 aa, Artificial Sequence
  Synthetic peptide
  MOD_RES (3)..(3): Any non-natural amino acid with an olefinic side chain
  SITE (3)..(7): /note=“May or may not be cross-linked via a side chain”
  MOD_RES (7)..(7): Any non-natural amino acid with an olefinic side chain
  MOD_RES (13)..(13): Any non-natural amino acid with an olefinic side chain
  SITE (13)..(17): /note=“May or may not be cross-linked via a side chain”
  MOD_RES (17)..(17): Any non-natural amino acid with an olefinic side chain
ETXDFLXEWFDVXAEDXDYL

SEQ ID NO: 133 - protein, 20 aa, Artificial Sequence
  Synthetic peptide
  MOD_RES (3)..(3): Any non-natural amino acid with an olefinic side chain
  SITE (3)..(7): /note=“May or may not be cross-linked via a side chain”
  MOD_RES (7)..(7): Any non-natural amino acid with an olefinic side chain
  MOD_RES (13)..(13): Any non-natural amino acid with an olefinic side chain
  SITE (13)..(17): /note=“May or may not be cross-linked via a side chain”
  MOD_RES (17)..(17): Any non-natural amino acid with an olefinic side chain
ETXDFYXEWFDVXAEDXDYL

SEQ ID NO: 134 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
IEEQXKTFXDKFNHEAEDLFYQSS

SEQ ID NO: 135 - protein, 24 aa, Artificial Sequence
  Synthetic peptide
  MOD_RES (8)..(8): Any non-natural amino acid with an olefinic side chain
  SITE (8)..(12): /note=“May or may not be cross-linked via a side chain”
  MOD_RES (12)..(12): Any non-natural amino acid with an olefinic side chain
IEEQAKTXLDKXNHEAEDLFYQSS

SEQ ID NO: 136 - protein, 24 aa, Artificial Sequence
  Synthetic peptide
  MOD_RES (12)..(12): Any non-natural amino acid with an olefinic side chain
  SITE (12)..(16): /note=“May or may not be cross-linked via a side chain”
  MOD_RES (16)..(16): Any non-natural amino acid with an olefinic side chain
IEEQAKTFLDKXNHEXEDLFYQSS

SEQ ID NO: 137 - protein, 24 aa, Artificial Sequence
  Synthetic peptide
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
IEEQAKTFLDKFNHEXEDLXYQSS

SEQ ID NO: 138 - protein, 24 aa, Artificial Sequence
  Synthetic peptide
  MOD_RES (9)..(9): Any non-natural amino acid with an olefinic side chain
  SITE (9)..(16): /note=“May or may not be cross-linked via a side chain”
  MOD_RES (16)..(16): Any non-natural amino acid with an olefinic side chain
IEEQAKTFXDKFNHEXEDLFYQSS

SEQ ID NO: 139 - protein, 24 aa, Artificial Sequence
  Synthetic peptide
  MOD_RES (5)..(5): Any non-natural amino acid with an olefinic side chain
  SITE (5)..(12): /note=“May or may not be cross-linked via a side chain”
  MOD_RES (12)..(12): Any non-natural amino acid with an olefinic side chain
IEEQXKTFLDKXNHEAEDLFYQSS

SEQ ID NO: 140 - protein, 24 aa, Artificial Sequence
  Synthetic peptide
  MOD_RES (12)..(12): Any non-natural amino acid with an olefinic side chain
  SITE (12)..(19): /note=“May or may not be cross-linked via a side chain”
  MOD_RES (19)..(19): Any non-natural amino acid with an olefinic side chain
IEEQAKTFLDKXNHEAEDXFYQSS

SEQ ID NO: 141 - protein, 24 aa, Artificial Sequence
  Synthetic peptide
  MOD_RES (1)..(1): Any non-natural amino acid with an olefinic side chain
  SITE (1)..(5): /note=“May or may not be cross-linked via a side chain”
  MOD_RES (5)..(5): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
XEEQXKTFLDKFNHEXEDLXYQSS

SEQ ID NO: 142 - protein, 24 aa, Artificial Sequence
  Synthetic peptide
  MOD_RES (1)..(1): Any non-natural amino acid with an olefinic side chain
  SITE (1)..(5): /note=“May or may not be cross-linked via a side chain”
  MOD_RES (5)..(5): Any non-natural amino acid with an olefinic side chain
  MOD_RES (12)..(12): Any non-natural amino acid with an olefinic side chain
  SITE (12)..(16): /note=“May or may not be cross-linked via a side chain”
  MOD_RES (16)..(16): Any non-natural amino acid with an olefinic side chain
XEEQXKTFLDKXNHEXEDLFYQSS

SEQ ID NO: 143 - protein, 24 aa, Artificial Sequence
  Synthetic peptide
  MOD_RES (5)..(5): Any non-natural amino acid with an olefinic side chain
  SITE (5)..(9): /note=“May or may not be cross-linked via a side chain”
  MOD_RES (9)..(9): Any non-natural amino acid with an olefinic side chain
  MOD_RES (16)..(16): Any non-natural amino acid with an olefinic side chain
  SITE (16)..(20): /note=“May or may not be cross-linked via a side chain”
  MOD_RES (20)..(20): Any non-natural amino acid with an olefinic side chain
IEEQXKTFXDKFNHEXEDLXYQSS

SEQ ID NO: 144 - protein, 24 aa, Artificial Sequence
  Synthetic peptide
  MOD_RES (1)..(1): Any amino acid
  MOD_RES (8)..(8): Any amino acid
  MOD_RES (9)..(9): Any amino acid
  MOD_RES (12)..(12): Any amino acid
  MOD_RES (13)..(13): Any amino acid
  MOD_RES (19)..(19): Any amino acid
  MOD_RES (20)..(20): Any amino acid
  MOD_RES (23)..(23): Any amino acid
  MOD_RES (24)..(24): Any amino acid
XEEQAKTXXDKXXHEAEDXXYQXX

SEQ ID NO: 145 - protein, 24 aa, Artificial Sequence
  Synthetic peptide
  VARIANT (1)..(1): /replace=“A”
  SITE (1)..(24): /note=“Variant residues given in the sequence have no preference with respect to those in the annotations for variant positions”
  VARIANT (8)..(8): /replace=“A”
  VARIANT (9)..(9): /replace=“A”
  VARIANT (12)..(12): /replace=“A”
  VARIANT (13)..(13): /replace=“A”
  VARIANT (18)..(18): /replace=“Q”
  VARIANT (19)..(19): /replace=“A”
  VARIANT (20)..(20): /replace=“A”
  VARIANT (23)..(23): /replace=“A”
  VARIANT (24)..(24): /replace=“A”
IEEQAKTFLDKFNHEAEDLFYQSS

SEQ ID NO: 146 - protein, 24 aa, Artificial Sequence
  Synthetic peptide
  SITE (1)..(24): /note=“Variant residues given in the sequence have no preference with respect to those in the annotations for variant positions”
  VARIANT (18)..(18): /replace=“Q”
  VARIANT (22)..(22): /replace=“Y”
IEEQAKTFLDKFNHEAEDLFYQSS

SEQ ID NO: 147 - protein, 24 aa, Artificial Sequence
  Synthetic peptide
  SITE (1)..(24): /note=“Variant residues given in the sequence have no preference with respect to those in the annotations for variant positions”
  VARIANT (7)..(7): /replace=“E”
  VARIANT (18)..(18): /replace=“Q”
IEEQAKTFLDKFNHEAEDLFYQSS

SEQ ID NO: 148 - protein, 24 aa, Artificial Sequence
  Synthetic peptide
  SITE (1)..(24): /note=“Variant residues given in the sequence have no preference with respect to those in the annotations for variant positions”
  VARIANT (7)..(7): /replace=“F”
  VARIANT (18)..(18): /replace=“Q”
IEEQAKTFLDKFNHEAEDLFYQSS

SEQ ID NO: 149 - protein, 24 aa, Artificial Sequence
  Synthetic peptide
  VARIANT (1)..(1): /replace=“A” or “Any stapling amino acid”
  SITE (1)..(24): /note=“Variant residues given in the sequence have no preference with respect to those in the annotations for variant positions”
  VARIANT (5)..(5): /replace=“Any stapling amino acid”
  VARIANT (7)..(7): /replace=“E” or “F”
  VARIANT (8)..(8): /replace=“A” or “Any stapling amino acid”
  VARIANT (9)..(9): /replace=“A” or “Any stapling amino acid”
  VARIANT (12)..(12): /replace=“A” or “Any stapling amino acid”
  VARIANT (13)..(13): /replace=“A”
  VARIANT (16)..(16): /replace=“Any stapling amino acid”
  VARIANT (18)..(18): /replace=“Q”
  VARIANT (19)..(19): /replace=“A” or “Any stapling amino acid”
  VARIANT (20)..(20): /replace=“A” or “Any stapling amino acid”
  VARIANT (22)..(22): /replace=“Y”
  VARIANT (23)..(23): /replace=“A”
  VARIANT (24)..(24): /replace=“A”
IEEQAKTFLDKFFHEAEDLFYQSS

SEQ ID NO: 150 - protein, 19 aa, Artificial Sequence
  Synthetic peptide
KTFLDKFNHEAEDLFYQSS

SEQ ID NO: 151 - protein, 28 aa, Artificial Sequence
  Synthetic peptide
  VARIANT (1)..(1): /replace=“T” or “V” or “ ”
  SITE (1)..(28): /note=“Variant residues given in the sequence have no preference with respect to those in the annotations for variant positions”
  VARIANT (3)..(3): /replace=“D”
  VARIANT (4)..(4): /replace=“N” or “K” or “R” or “L”
  VARIANT (5)..(5): /replace=“V”
  VARIANT (6)..(6): /replace=“E” or “R”
  VARIANT (7)..(7): /replace=“S” or “E”
  VARIANT (10)..(10): /replace=“N” or “E”
  VARIANT (11)..(11): /replace=“N”
  VARIANT (13)..(13): /replace=“D”
  VARIANT (14)..(14): /replace=“Q” or “E” or “L” or “Y”
  VARIANT (17)..(17): /replace=“S”
  VARIANT (18)..(18): /replace=“E”
  VARIANT (19)..(19): /replace=“I”
  VARIANT (20)..(20): /replace=“S” or “M” or “Norleucine” or “A”
  VARIANT (23)..(23): /replace=“Y”
  VARIANT (24)..(24): /replace=“T”
IEEQAKTFLDKFNHEAEDLFYQSSSSLA

SEQ ID NO: 152 - protein, 19 aa, Artificial Sequence
  Synthetic peptide
  SITE (1)..(19): /note=“Variant residues given in the sequence have no preference with respect to those in the annotations for variant positions”
  VARIANT (13)..(13): /replace=“Q”
KTFLDKFNHEAEDLFYQSS

SEQ ID NO: 153 - protein, 17 aa, Artificial Sequence
  Synthetic peptide
  VARIANT (16)..(16): /replace=“ ”
  VARIANT (17)..(17): /replace=“ ”
DKFNHEAEDLFYQSSLA

SEQ ID NO: 154 - protein, 15 aa, Artificial Sequence
  Synthetic peptide
DKFNHEAEDLFYQSS

SEQ ID NO: 155 - protein, 15 aa, Artificial Sequence
  Synthetic peptide
  SITE (1)..(15): /note=“Variant residues given in the sequence have no preference with respect to those in the annotations for variant positions”
  VARIANT (9)..(9): /replace=“Q”
DKFNHEAEDLFYQSS

SEQ ID NO: 156 - protein, 14 aa, Artificial Sequence
  Synthetic peptide
  VARIANT (13)..(13): /replace=“ ”
  VARIANT (14)..(14): /replace=“ ”
NHEAEDLFYQSSLA

SEQ ID NO: 157 - protein, 12 aa, Artificial Sequence
  Synthetic peptide
NHEAEDLFYQSS

SEQ ID NO: 158 - protein, 12 aa, Artificial Sequence
  Synthetic peptide
  SITE (1)..(12): /note=“Variant residues given in the sequence have no preference with respect to those in the annotations for variant positions”
  VARIANT (6)..(6): /replace=“Q”
NHEAEDLFYQSS

SEQ ID NO: 159 - protein, 8 aa, Artificial Sequence
  Synthetic peptide
EDLFYQSS

SEQ ID NO: 160 - protein, 8 aa, Artificial Sequence
  Synthetic peptide
  SITE (1)..(8): /note=“Variant residues given in the sequence have no preference with respect to those in the annotations for variant positions”
  VARIANT (2)..(2): /replace=“Q”
EDLFYQSS

SEQ ID NO: 161 - protein, 4 aa, Artificial Sequence
  Synthetic peptide
YQSS

SEQ ID NO: 162 - protein, 6 aa, Artificial Sequence
  Synthetic peptide
NHEAED

SEQ ID NO: 163 - protein, 5 aa, Artificial Sequence
  Synthetic peptide
FYQSS

SEQ ID NO: 164 - protein, 6 aa, Artificial Sequence
  Synthetic peptide
  SITE (1)..(6): /note=“Variant residues given in the sequence have no preference with respect to those in the annotations for variant positions”
  VARIANT (5)..(5): /replace=“A” or “ ”
  VARIANT (6)..(6): /replace=“ ”
YQSALA

SEQ ID NO: 165 - protein, 6 aa, Artificial Sequence
  Synthetic peptide
DHANHE

SEQ ID NO: 166 - protein, 17 aa, Artificial Sequence
  Synthetic peptide
  SITE (1)..(17): /note=“Variant residues given in the sequence have no preference with respect to those in the annotations for variant positions”
  VARIANT (16)..(16): /replace=“A” or “ ”
  VARIANT (17)..(17): /replace=“ ”
DKANHEAEDAAYQSALA

SEQ ID NO: 167 - protein, 80 aa, Mus musculus
MSSSSWLLLSLVAVTTAQSLTEENAKTFLNNFNQEAEDLSYQSSLASWNYNTNITEENAQKMSEAAAKWSAFYEEQSKTA

SEQ ID NO: 168 - protein, 80 aa, Rattus norvegicus
MSSSCWLLLSLVAVATAQSLIEEKAESFLNKFNQEAEDLSYQSSLASWNYNTNITEENAQKMNEAAAKWSAFYEEQSKIA

SEQ ID NO: 169 - protein, 79 aa, Danio rerio
MCARWLLLLALASVACCQTVEDRAREFLNKFDEEASDIMYQYTLASWAYNTDISQENADKEAEAYAIWSEYYNKMSEES

SEQ ID NO: 170 - protein, 80 aa, Sus scrofa
MSGSFWLLLSLIPVTAAQSTTEELAKTFLEKFNLEAEDLAYQSSLASWTINTNITDENIQKMNDARAKWSAFYEEQSRIA

SEQ ID NO: 171 - protein, 27 aa, Artificial Sequence
  Synthetic peptide
  MOD_RES (1)..(1): Beta-Ala
  SITE (1)..(26): /note=“Variant residues given in the sequence have no preference with respect to those in the annotations for variant positions”
  VARIANT (26)..(26): /replace=“A” or “ ”
  VARIANT (27)..(27): /replace=“ ”
XIEEQAKTAADKANHEAEQAAYQSALA

SEQ ID NO: 172 - protein, 24 aa, Artificial Sequence
  Synthetic peptide
  MOD_RES (1)..(1): Any non-natural amino acid with an olefinic side chain
  SITE (1)..(5): /note=“May or may not be cross-linked via a side chain”
  MOD_RES (5)..(5): Any non-natural amino acid with an olefinic side chain
XEEQXKTFLDKFNHEAEDLFYQSS